(19) United States
(12) Patent Application Publication
(10) Pub. No.: US 2024/0325336 A1
(43) Pub. Date: Oct. 3, 2024

(54) COMBINATION THERAPIES COMPRISING OXYGEN-CONTAINING STRUCTURALLY ENHANCED FATTY ACIDS FOR TREATMENT OF NON-ALCOHOLIC STEATOHEPATITIS

(71) Applicant: NORTHSEA THERAPEUTICS B.V., Amsterdam (NL)

(72) Inventor: David Alan FRASER, Kabelvag (NO)

(21) Appl. No.: 18/258,620

(22) PCT Filed: Dec. 21, 2021
(86) PCT No.: PCT/IB2021/062115

§ 371 Date: Jun. 21, 2023

Related U.S. Application Data
(60) Provisional application No. 63/128,996, filed on Dec. 22, 2020.

(51) Int. Cl. A61K 31/202; A61K 31/519; A61K 31/575; A61K 38/26; A61P 1/16

(52) U.S. Cl. CPC A61K 31/202; A61K 31/519; A61K 31/575; A61K 38/26; A61P 1/16

(57) ABSTRACT

The present disclosure provides a combination therapy for use in therapeutic and/or prophylactic treatment of non-alcoholic steatohepatitis (NASH) and/or alcoholic steatohepatitis (ASH), wherein the combination therapy comprises an unsaturated fatty acid with an oxygen incorporated in the β-position and an α-substituent and at least one additional active agent chosen from a glucagon-like peptide 1 receptor agonist, an acetyl-CoA carboxylase inhibitor, and a farnesoid X receptor agonist.

This application claims the benefit of priority of U.S. Provisional Application No. 63/128,996, filed Dec. 22, 2020, which is incorporated herein by reference in its entirety.

### FIELD

The present disclosure relates to combination therapies for treating non-alcoholic steatohepatitis (NASH), alcoholic steatohepatitis (ASH), and other hepatic disorders characterized by fibrosis and/or hepatic inflammation in a subject in need thereof. Further, the present disclosure relates to methods of treating non-alcoholic steatohepatitis (NASH), alcoholic steatohepatitis (ASH), and other hepatic disorders characterized by fibrosis and/or hepatic inflammation in a subject in need thereof using such combination therapies. The combination therapies comprise unsaturated fatty acids with an oxygen incorporated in the β-position and an α-substituent.

### BACKGROUND

Non-alcoholic fatty liver disease (NAFLD) and non-alcoholic steatohepatitis (NASH) are frequently used interchangeably despite the fact that NAFLD encompasses a much broader spectrum of liver disease including isolated hepatosteatosis (>5% of hepatocytes histologically). Hepatosteatosis is most likely a relatively benign disorder when not accompanied by an inflammatory response and cellular damage. However, a subgroup of NAFLD patients have liver cell injury and inflammation in addition to hepatosteatosis, a condition known as non-alcoholic steatohepatitis (NASH). NASH is virtually indistinguishable histologically from alcoholic steatohepatitis (ASH). While the simple steatosis seen in NAFLD does not correlate with increased short-term morbidity or mortality, NASH dramatically increases the risks of cirrhosis, liver failure, and hepatocellular carcinoma (HCC). Cirrhosis due to NASH is an increasingly frequent reason for liver transplantation. While the morbidity and mortality from liver causes are greatly increased in patients with NASH, they correlate even more strongly with the morbidity and mortality from cardiovascular disease.

Uniform criteria for diagnosing and staging NASH are still debated. Key histologic components of NASH are steatosis, hepatocellular ballooning, and lobular inflammation; fibrosis is not part of the histologic definition of NASH. However, the degree of fibrosis on liver biopsy (stage) is predictive of the prognosis, whereas the degree of inflammation and necrosis on liver biopsy (grade) are not.

As with NASH, alcoholic liver disease (ALD), also known as alcoholic fatty liver disease, can be grouped into histological stages representing a transition from fatty liver or simple steatosis to alcoholic hepatitis (i.e., ASH) and finally to chronic hepatitis with hepatic fibrosis or cirrhosis. Thus, although the origins of ASH and NASH may differ, the hepatic response to the respective chronic insult has many similarities, including the pro-inflammatory and pro-fibrotic cascades involving macrophage activation and cytokine production and the resultant activated stellate cells, i.e., proliferating myofibroblasts. (See, e.g., Friedman, SL; Alcoholism: Clinical and Experimental Research. 1999 May; 23(5):904-910.)

With respect to the various histological components, treatment with omega-3 fatty acids have been shown to effectively reduce hepatosteatosis in patients with NAFLD (Scorletti E, et al., Effects of purified eicosapentaenoic and docosahexanoic acids in non-alcoholic fatty liver disease: Results from the *WELCOME study, Hepatology. 2014 October; 60(4):1211-2). Such studies suggest that if treatment is established at an early stage of the disease, it may conceivably slow progression to the latter more severe stages of the disease. However, it is questionable whether omega-3 fatty acids are sufficiently potent to treat and/or reverse NASH where pronounced histological/inflammatory changes have developed (Sanyal A J, et al; EPE-A Study Group, Gastroenterology. 2014 August; 147(2):377-84.e1).

In particular, targeting the fibrosis component of NASH and ASH is relevant to successfully treating progressed forms of these indications as hepatic fibrosis can further progress to cirrhosis, which in turn is associated with a highly increased morbidity and mortality. It also represents the major hard endpoint in clinical studies of chronic liver diseases. For example, emerging data suggests fibrosis, rather than NASH per se, to be the most important histological predictor of both liver and non-liver related death. Additionally, cirrhosis is a strong cofactor of primary liver cancer.

WO2016173923A1 discloses that sulphur-containing structurally modified fatty acids like 2-ethyl-2-((5Z,8Z,11Z,14Z,17Z)-icosa-5,8,11,14,17-pentaenylthio)butanoic acid (Compound N) may be useful in the treatment of NASH. This was based on the finding that Compound N was superior to rosiglitazone, a PPAR-gamma agonist, in preventing diet-induced hepatic fibrosis. It also indicated that Compound N prevented the influx of inflammatory cells into the liver. It was demonstrated that Compound N was effective in reducing fibrosis (as measured by hydroxyproline/proline ratio) in APOE*3Leiden.CETP mice, a model that develops a very mild heaptic fibrotic response.

WO2019111048 of BASF AS, the contents of which are incorporated by reference herein, discloses that oxygen-containing structurally modified fatty acids like 2-(((5Z,8Z,11Z,14Z,17Z)-icosa-5,8,11,14,17-pentaen-1-yl)oxy)butanoic acid (Compound A) may be useful in the treatment of NASH and ASH. Compound A was found to address multiple aspects of NASH, including hepatic steatosis, inflammation, and fibrosis. These findings were replicated in multiple rodent NASH models that ranged in severity of inflammation and fibrosis, including a APOE*3Leiden.CETP high-fat diet mouse model, an obese diet-induced NASH mouse model (ob/ob AMLN high-fat fed), a streptozotocin injection/high-fat diet induced NASH (STAM) mouse model, and a methionine and choline deficient diet induced NASH (CDAA/high-fat diet) mouse model.

Research in both humans and animal models of NASH have convincingly demonstrated that there are multiple factors involved in the development of steatohepatitis and fibrosis as opposed to isolated hepatosteatosis. These include insulin resistance, oxidative stress, inflammation, gut-derived endotoxin and excessive hepatic cholesterol and bile acids. All of these factors have been shown to play an important contributing role in genetically susceptible individuals and accordingly drugs targeting these pathways are being developed for the treatment of NASH. The complexity of the etiopathogenesis of NASH is also related to the involvement of extra-hepatic organs. For example, gut-derived inflammatory cytokines/endotoxin can serve as a potent pro-inflammatory stimuli. Additionally, both insulin resistant visceral and peripheral adipose can flood the already sensitised liver with free-fatty acids and pro-inflammatory adipokines.

Based on the complexity of NASH and ASH, combination therapeutics may be desirable for targeting different pathways contributing to these diseases.

### BRIEF SUMMARY

The present disclosure provides a combination therapy comprising a structurally enhanced fatty acid containing oxygen and one or more additional active agents for the therapeutic and/or prophylactic treatment of non-alcoholic steatohepatitis (NASH) and/or alcoholic steatohepatitis (ASH). The present disclosure likewise provides methods of treating NASH and/or ASH in a subject in need thereof comprising administering an oxygen-containing structurally modified fatty acid and at least one additional active agent.

The present disclosure provides a combination therapy comprising a first compound of Formula (II)

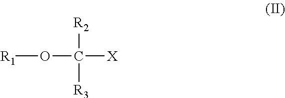

wherein R₁ is selected from a C₁₀-C₂₂ alkenyl having 3-6 double bonds;
R₂ and R₃ are the same or different and are selected from the group of a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group; wherein R₂ and R₃ can be connected in order to form a cycloalkane like cyclopropane, cyclobutane, cyclopentane or cyclohexane;
X is a carboxylic acid or a derivative thereof; wherein the derivative is a carboxylate, such as a carboxylic ester; a glyceride; an anhydride; a carboxamide; a phospholipid; or a hydroxymethyl; or a prodrug thereof;
or a pharmaceutically acceptable salt, solvate, or solvate of such a salt thereof, and
at least one additional active agent chosen from a glucagon-like peptide 1 (GLP-1) receptor agonist, an acetyl-CoA carboxylase (ACC) inhibitor, and a farnesoid X receptor (FXR) agonist, for use in therapeutic and/or prophylactic treatment of non-alcoholic steatohepatitis (NASH) and/or alcoholic steatohepatitis (ASH).

The present disclosure likewise relates to a method of treating NASH and/or ASH in a subject in need thereof, comprising administering to the subject a pharmaceutically effective amount of a first compound of Formula (II):

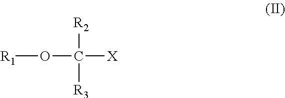

wherein R₁, R₂, R₃, and X are defined as described above,
and further comprising administering at least one additional active agent chosen from a GLP-1 receptor agonist, an ACC inhibitor, and a FXR agonist.

In some embodiments, the GLP-1 receptor agonist is semaglutide, the ACC inhibitor is firsocostat, and the FXR agonist is obeticholic acid (OCA).

In some embodiments, in the first compound R₂ and R₃ are the same or different and may be selected from a group of substituents consisting of a hydrogen atom, an alkyl group, an alkoxy group, an alkenyl group; or R₂ and R₃ can be connected in order to form a cycloalkane like cyclopropane, cyclobutane, cyclopentane or cyclohexane;
X represents a carboxylic acid or a derivative thereof, wherein the derivative is a carboxylic ester, a glyceride or a phospholipid;
or a pharmaceutically acceptable salt, solvate, or solvate of such a salt thereof.

The present disclosure also relates to a combination therapy comprising a first compound of Formula (I):

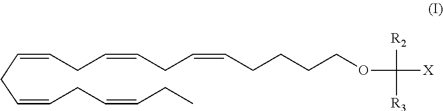

wherein R₂ and R₃ and X are defined as for Formula II. More particularly,
R₂ and R₃ are independently chosen from a hydrogen atom or linear, branched, and/or cyclic C₁-C₆ alkyl groups;
X is a carboxylic acid or a derivative thereof, wherein the derivative is a carboxylic ester, a glyceride, or a phospholipid;
or a pharmaceutically acceptable salt, solvate, or solvate of such a salt thereof, and at least one additional active agent chosen from a GLP-1 receptor agonist, an ACC inhibitor, and a FXR agonist,
for use in the therapeutic and/or prophylactic treatment of NASH and/or ASH. In some embodiments, the GLP-1 receptor agonist is semaglutide, the ACC inhibitor is firsocostat, and the FXR agonist is obeticholic acid (OCA).

Likewise, the present disclosure relates to a method of treating NASH and/or ASH in a subject in need thereof, comprising administering a pharmaceutically effective amount of a first compound of Formula (I):

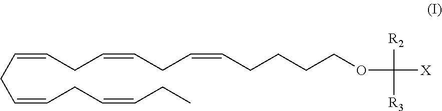

wherein R₂ and R₃ and X are defined as described above, and further comprising administering at least one additional active agent chosen from a GLP-1 receptor agonist, an ACC inhibitor, and a FXR agonist. In some embodiments, the compound of Formula (I) is 2-(((5Z,8Z,11Z,14Z,17Z)-icosa-5,8,11,14,17-pentaen-1-yl)oxy)butanoic acid (Compound A):

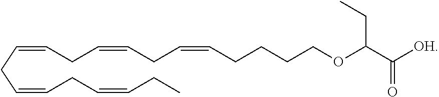 [SMILES: CC/C=C\C/C=C\C/C=C\C/C=C\C/C=C\CCCCOC(CC)C(=O)O]

The present disclosure also provides a combination therapy comprising 2-(((5Z,8Z,11Z,14Z,17Z)-icosa-5,8,11,14,17-pentaen-1-yl)oxy)butanoic acid (Compound A):

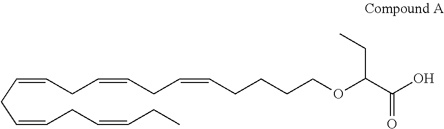 [SMILES: CC/C=C\C/C=C\C/C=C\C/C=C\C/C=C\CCCCOC(CC)C(=O)O]

or a pharmaceutically acceptable salt or ester thereof,
and at least one additional active agent chosen from a GLP-1 receptor agonist, an ACC inhibitor, and a FXR agonist,
for use in the therapeutic and/or prophylactic treatment of NASH and/or ASH. In some embodiments, the GLP-1 receptor agonist is semaglutide, the ACC inhibitor is firsocostat, and the FXR agonist is obeticholic acid (OCA).

The present disclosure also relates to the effects of the combination therapies in subjects with NASH and/or ASH. In some embodiments, the use of the combination therapy decreases the level of plasma and/or liver triglycerides in the subject. In some embodiments, the use of the combination therapy decreases the level of plasma and/or liver cholesterol in the subject. In some embodiments, the use of the combination therapy reduces hepatic steatosis in the subject. In some embodiments, the use of the combination therapy reduces hepatic inflammation in the subject. In some embodiments, the use of the combination therapy reduces hepatic fibrosis in the subject. In some embodiments, the use of the combination therapy reverses steatohepatitis in the subject.

### BRIEF DESCRIPTION OF DRAWINGS

FIGS. 1A-1B depict the effects of 8 weeks of administration of Compound A, semaglutide, firsocostat, OCA, and combinations thereof, on the relative body weight (FIG. 1A) and liver weight (FIG. 1B) in a CDAA/high-fat diet mouse model. The same experimental conditions apply to the results depicted in the remaining figures.

FIGS. 2A-2B depict the effects of Compound A, semaglutide, firsocostat, OCA, and combinations thereof, on the levels of plasma alanine transaminase (ALT) (FIG. 2A) and plasma aspartate transaminase (AST) (FIG. 2B).

FIGS. 3A-3B depict the effects of Compound A, semaglutide, firsocostate, OCA, and combinations thereof, on plasma triglycerides (TG) (FIG. 3A) and plasma total cholesterol (TC) (FIG. 3B).

FIGS. 4A-4B depict the effects of Compound A, semaglutide, firsocostat, OCA, and combinations thereof, on the relative levels of liver TC (FIG. 4A) and liver TG (FIG. 4B).

FIGS. 5A-5B depict the effects of Compound A, semaglutide, firsocostat, OCA, and combinations thereof, on the hepatic steatosis area (relative levels of liver lipids) (FIG. 5A) and the percentage of hepatocytes with lipid droplets (FIG. 5B).

FIG. 6 depicts the effects of Compound A, semaglutide, firsocostat, OCA, and combinations thereof, on hepatic steatosis scores.

FIGS. 7A-7B depict the effects of Compound A, semaglutide, firsocostat, OCA, and combinations thereof on the number of hepatic inflammatory cells (FIG. 7A) and hepatic inflammatory foci (FIG. 7B).

FIGS. 8A-8B depict the effects of Compound A, semaglutide, firsocostat, OCA, and combinations thereof, on lobular inflammation (FIG. 8A) and NAFLD activity scores (FIG. 8B).

FIGS. 9A-9B depict the effects of Compound A, semaglutide, firsocostat, OCA, and combinations thereof, on the relative levels of liver hydroxyproline (HP) (FIG. 9A) and hepatic area of galectin-3 expression (FIG. 9B).

FIGS. 10A-10C depicts the effects of Compound A, semaglutide, firsocostat, OCA, and combinations thereof, on the hepatic fibrotic area determined by picrosirius red (PSR) (FIG. 10A), hepatic sinusoidal fibrotic area determined by PSR (FIG. 10B) and hepatic periportal fibrotic area determined by PSR (FIG. 10C).

FIGS. 11A-11B depicts the effects of Compound A, semaglutide, firsocostat, OCA, and combinations thereof, on the hepatic area of collegen 1a1 (Col1a1) expression (FIG. 11A) and hepatic area of α-smooth muscle actin (α-SMA) expression (FIG. 11B).

FIG. 12 depicts the effects of Compound A, semaglutide, firsocostat, OCA, and combinations thereof on the hepatic fibrosis stage.

FIGS. 13A-131 shows representative images of liver morphology at termination, using Sirius Red staining (magnification 20×, scale bar=100 μm).

FIGS. 14A-14I shows representative images of liver morphology at termination, using H&E staining (magnification 20×, scale bar=100 μm).

### DETAILED DESCRIPTION

It should be noted that embodiments and features described in the context of one aspect of the present disclosure also apply to the other aspects of the disclosure. Particularly, the embodiments applying to the method of treating non-alcoholic steatohepatitis or alcohol steatohepatitis according to the present disclosure also apply to the aspect directed to compounds and/or combination therapies for use in treating non-alcoholic steatohepatitis or alcohol steatohepatitis, all according to the present disclosure.

Particular aspects of the disclosure are described in greater detail below. The terms and definitions as used in the present application and as clarified herein are intended to represent the meaning within the present disclosure.

The singular forms “a,” “an,” and “the” include plural reference unless the context dictates otherwise.

The terms “approximately” and “about” mean to be nearly the same as a referenced number or value. As used herein, the terms “approximately” and “about” should be generally understood to encompass ±5% of a specified amount, frequency, or value, unless otherwise specified.

The terms “treat,” “treating,” and “treatment” include any therapeutic or prophylactic application that can benefit a human or non-human mammal. Both human and veterinary treatments are within the scope of the present disclosure. Treatment may be responsive to an existing condition or it may be prophylactic, i.e., preventative.

The terms “preventing and/or treating” and “therapeutic and/or prophylactic treatment of” may interchangeably be used. Typically, the compounds and/or combination therapies of the present disclosure will be used for treating, i.e., therapeutic treatment of NASH or ASH. However, it is also foreseen that in some cases the compositions will be used for prophylactic treatment of NASH or ASH, for example in cases where a patient has one or multiple risk factors associated with NASH or ASH.

The terms “reverse” and “regress,” and variants thereof (e.g., reversal, regression) are used herein with respect to treatment that reduces the severity of an existing condition, or a parameter of that condition, to a more favorable level than the level at the start of treatment.

The terms “administer,” “administration,” and “administering” as used herein refer to (1) providing, giving, dosing and/or prescribing by either a health practitioner or his authorized agent or under his direction compounds and/or combination therapies according to the present disclosure, and (2) putting into, taking or consuming by the human patient or person himself or herself, or non-human mammal compositions according to the present disclosure.

The terms “administered in combination” and “co-administration” or “coadministration” are used interchangeably and refer to administration of a (a) compound of Formula (I) or (II), or a pharmaceutically acceptable salt, solvate, or solvate of such a salt of any of the foregoing; and (b) at least one additional active agent, together in a coordinated fashion. For example, the co-administration can be simultaneous administration, sequential administration, overlapping administration, interval administration, continuous administration, or a combination thereof. The mode of administration may be different for the compounds and the additional agent(s), and the co-administration includes any mode of administration, such as oral, subcutaneous, sublingual, transmucosal, parenteral, intravenous, intra-arterial, intra-peritoneal, buccal, sublingual, topical, vaginal, rectal, ophthalmic, otic, nasal, inhaled, and transdermal, or a combination thereof. Examples of the parenteral administration include but are not limited to intravenous (IV) administration, intraarterial administration, intramuscular administration, subcutaneous administration, intraosseous administration, intrathecal administration, or a combination thereof. The compound of Formula (I) or (II) and the additional active agent can be independently administered, e.g., orally or parenterally. In some embodiments, the compound of Formula (I) or (II) and the additional active agent are both administered orally. In some embodiment, the compound of Formula (I) or (II) is administered orally; and the additional active agent is administered parenterally. The parenteral administration may be conducted via injection or infusion. In some embodiments, the method and/or use of the present disclosure are directed to the therapeutic and/or prophylactic treatment of NASH or ASH using at least two different active agents, the compound of Formula (I) or (II), and an additional active agent, respectively. The at least two active agents can be seen as a “combined product” or “combination therapy”, wherein the agents are e.g., separately packed and wherein both agents are required to achieve the optimal intended effect.

The term “pharmaceutically effective amount” means an amount sufficient to achieve the desired pharmacological and/or therapeutic effects, i.e., an amount of the disclosed compound that is effective for its intended purpose, and is interchangeable with the term “therapeutically effective amount.” While individual subject/patient needs may vary, the determination of optimal ranges for effective amounts of the disclosed compound is within the skill of the art. Generally, the dosage regimen for treating a disease and/or condition with the compounds and/or combination therapies presently disclosed may be determined according to a variety of factors such as the type, age, weight, sex, diet, and/or medical condition of the subject/patient.

The term “pharmaceutical composition” means a compound or combination of compounds according to the present disclosure in any form suitable for medical use.

The compounds of Formula (I) and Formula (II) of the present disclosure may exist in various stereoisomeric forms, including enantiomers, diastereomers, or mixtures thereof. It will be understood that the present disclosure encompasses all optical isomers of the compounds of Formula (I) and (II) as well as mixtures thereof. Hence, compounds of Formula (I) and (II) of the present disclosure that exist as diastereomers, racemates, and/or enantiomers are within the scope of the present disclosure.

In some embodiments, the combination therapies of the present disclosure comprise a first compound of Formula (II)

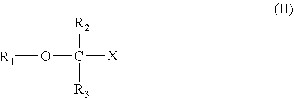

wherein R₁ is selected from a C₁₀-C₂₂ alkenyl having 3-6 double bonds;
R₂ and R₃ are the same or different and may be selected from a group of substituents consisting of a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group, where R₂ and R₃ can be connected in order to form a cycloalkane like cyclopropane, cyclobutane, cyclopentane or cyclohexane;
X represents a carboxylic acid or a derivative thereof, wherein the derivative is a carboxylate, such as a carboxylic ester; a glyceride; an anhydride; a carboxamide; a phospholipid; or a hydroxymethyl; or a prodrug thereof; or a pharmaceutically acceptable salt, solvate, or solvate of such a salt thereof; and at least one additional active agent, for use in therapeutic and/or prophylactic treatment of non-alcoholic steatohepatitis (NASH) and/or alcoholic steatohepatitis (ASH).

The present disclosure is likewise directed to combination therapies comprising a first compound of Formula (II)

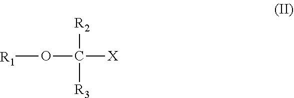

wherein R₁ is selected from a C₁₀-C₂₂ alkenyl having 3-6 double bonds;
R₂ and R₃ are the same or different and may be selected from a group of substituents consisting of a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group, where R₂ and R₃ can be connected in order to form a cycloalkane like cyclopropane, cyclobutane, cyclopentane or cyclohexane;
X represents a carboxylic acid or a derivative thereof, wherein the derivative is a carboxylate, such as a carboxylic ester; a glyceride; an anhydride; a carboxamide; a phospholipid; or a hydroxymethyl; or a prodrug thereof; or a pharmaceutically acceptable salt, solvate, or solvate of such a salt thereof; and
at least one additional active agent, for use in therapeutic and/or prophylactic treatment of fatty liver disease. In some embodiments, the fatty liver disease is non-alcoholic fatty liver disease (NAFLD). In some embodiments, the fatty liver disease is alcoholic fatty liver disease (ALD). In some embodiments, the fatty liver disease is not accompanied by an inflammatory response and cellular damage.

In some embodiments, the at least one additional active agent is selected from a glucagon-like peptide 1 (GLP-1) receptor agonist, an acetyl-CoA carboxylase (ACC) inhibitor, and a farnesoid X receptor (FXR) agonist.

In some embodiments, the at least one additional active agent is selected from a GLP-1 receptor agonist. In some embodiments, the at least one additional active agent is selected from an ACC inhibitor. In some embodiments, the at least one additional active agent is selected from a FXR agonist.

In some embodiments, the GLP-1 receptor agonist is semaglutide. In some embodiments, the ACC inhibitor is firsocostat. In some embodiments, the FXR agonist is obeticholic acid (OCA).

In some embodiments, the at least one additional active agent is semaglutide. In some embodiments, the at least one additional active agent is firsocostat. In some embodiments, the at least one additional active agent is obeticholic acid (OCA).

In some embodiments, for the first compound R₁ is a C₁₈-C₂₂ alkenyl having 3-6 double bonds, such as 5 or 6 double bonds. In some embodiments one double bond is in the omega-3 position.

In preferred embodiments, for the first compound R₂ and R₃ are independently chosen from a hydrogen atom and linear, branched, and/or cyclic C₁-C₆ alkyl groups. In some embodiments, at least one of R₂ and R₃ is chosen from a hydrogen atom, a methyl group, an ethyl group, an n-propyl group, an isopropyl group, a butyl group and a pentyl group.

In some embodiments, for the first compound X represents a carboxylic acid or a carboxylic ester; or a pharmaceutically acceptable salt, solvate, or solvate of such a salt thereof.

In some embodiments, there is provided a method of treating NASH and/or ASH in a subject in need thereof, the method comprising administering to the subject a pharmaceutically effective amount of a first compound of Formula (II):

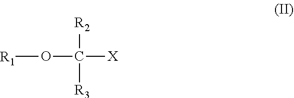

wherein R₁, R₂, R₃, and X are defined as described above; or prodrug thereof; or a pharmaceutically acceptable salt, solvate, or solvate of such salt thereof; and at least one additional active agent. In some embodiments, the at least one additional active agent is selected from a glucagon-like peptide 1 (GLP-1) receptor agonist, an acetyl-CoA carboxylase (ACC) inhibitor, and a farnesoid X receptor (FXR) agonist.

In some embodiments, the present disclosure provides a method of treating non-alcoholic steatohepatitis or alcoholic steatohepatitis in a subject in need thereof, comprising administering to the subject a combination therapy comprising a pharmaceutically effective amount of a first compound of Formula (I):

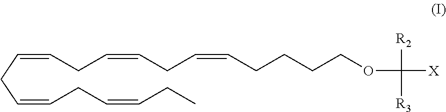

wherein R₂, R₃ and X are defined as for Formula (II) and at least one additional active agent selected from a GLP-1 receptor agonist, an ACC inhibitor, and a FXR agonist. In some embodiments, the GLP-1 receptor agonist is semaglutide, the ACC inhibitor is firsocostat, and the FXR agonist is obeticholine.

In some embodiments, for compounds of Formula (I), R₂ and R₃ are independently chosen from a hydrogen atom or linear, branched, and/or cyclic C₁-C₆ alkyl groups; and
X is a carboxylic acid or a carboxylic ester; or a pharmaceutically acceptable salt, solvate, or solvate of such a salt of any of the foregoing.

The present disclosure similarly provides for methods of treating fatty liver disease in a subject in need thereof, comprising to the subject a combination therapy comprising a pharmaceutically effective amount of a first compound of Formula (I) or (II), wherein R₁, R₂, R₃, and X are defined as described above, and at least one additional active agent selected from a GLP-1 receptor agonist, an ACC inhibitor, and a FXR agonist. In some embodiments, the GLP-1 receptor agonist is semaglutide, the ACC inhibitor is firsocostat, and the FXR agonist is obeticholine. In some embodiments, the fatty liver disease is non-alcoholic fatty liver disease (NAFLD). In some embodiments, the fatty liver disease is alcoholic fatty liver disease (ALD). In some embodiments, the fatty liver disease is not accompanied by an inflammatory response and cellular damage.

In some embodiments, the present disclosure provides a combination therapy comprising a first compound of Formula (I):

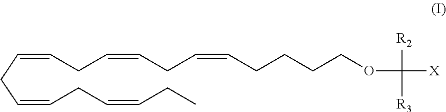

wherein R₂, R₃ and X are defined as for Formula (II) and at least one additional active agent selected from a GLP-1 receptor agonist, an ACC inhibitor, and a FXR agonist, for use in treating NASH and/or ASH. In some embodiments, the GLP-1 receptor agonist is semaglutide, the ACC inhibitor is firsocostat, and the FXR agonist is obeticholine.

In some embodiments, the present disclosure provides a combination therapy comprising a first compound of Formula (I):

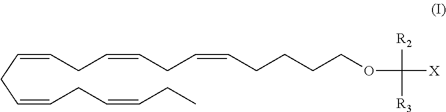

wherein R₂, R₃ and X are defined as for Formula (II) and at least one additional active agent selected from a GLP-1 receptor agonist, an ACC inhibitor, and a FXR agonist, for use in treating fatty liver disease. In some embodiments, the GLP-1 receptor agonist is semaglutide, the ACC inhibitor is firsocostat, and the FXR agonist is obeticholine. In some embodiments, In some embodiments, the fatty liver disease is non-alcoholic fatty liver disease (NAFLD). In some embodiments, the fatty liver disease is alcoholic fatty liver disease (ALD). In some embodiments, the fatty liver disease is not accompanied by an inflammatory response and cellular damage.

In some embodiments, for compounds of Formula (I), R₂ and R₃ are independently chosen from a hydrogen atom or linear, branched, and/or cyclic C₁-C₆ alkyl groups;
X is a carboxylic acid or a carboxylic ester; or a pharmaceutically acceptable salt, solvate, or solvate of such a salt of any of the foregoing.

In those cases where R₂ and R₃ are different, the compounds of Formula (I) and Formula (II) are capable of existing in stereoisomeric forms. It will be understood that the present disclosure encompasses all optical isomers of the compounds of Formula (I) and Formula (II) and mixtures thereof.

In at least one embodiment, R₂ and R₃ are independently selected from the group of a hydrogen atom, a methyl group, an ethyl group, a n-propyl group, an isopropyl group, a butyl group and a pentyl group.

In at least one embodiment, R₂ and R₃ are independently selected from the group of a hydrogen atom, a methyl group, and an ethyl group.

In at least one embodiment, one of R₂ and R₃ is a hydrogen atom and the other one of R₂ and R₃ is chosen from a C₁-C₃ alkyl group. In one embodiment, one of R₂ and R₃ is a hydrogen atom and the other one of R₂ and R₃ is selected from a methyl group and an ethyl group. In some embodiments, one of R₂ and R₃ is a hydrogen atom and the other one is an ethyl group.

For compounds of both Formula (I) and Formula (II), R₂ and R₃ are, in some embodiments, independently C₁-C₆ alkyl groups. In some embodiments both R₂ and R₃ are C₁-C₃ alkyl groups. In some embodiments R₂ and R₃ are the same or different and each are independently chosen from a methyl group, an ethyl group, an n-propyl group, and an isopropyl group. In some embodiments R₂ and R₃ are the same and are selected from a pair of methyl groups, a pair of ethyl groups, a pair of n-propyl groups and a pair of isopropyl groups. In at least one embodiment R₂ and R₃ are ethyl groups. In some embodiments, one of R₂ and R₃ is a methyl group and the other one is an ethyl group. In some embodiments, one of R₂ and R₃ is an ethyl group and the other one is a n-propyl group.

In at least one embodiment, the compounds of Formula (I) or (II) are present in their various stereoisomeric forms, such as an enantiomer (R or S), a diastereomer, or mixtures thereof. In at least one embodiment, the compounds are present in racemic form.

In cases where the compound according to Formula (I) or (II) is a salt of a counter-ion with at least one stereogenic center, or ester of an alcohol with at least one stereogenic center, the compound may have multiple stereocenters. In those situations, the compounds of the present disclosure may exist as diastereomers. Thus, in at least one embodiment, the compounds of the present disclosure are present as at least one diastereomer.

In some embodiments, the compound of Formula (II) or (I) is administered in combination with firsocostat. In some embodiments, the compound of Formula (II) or (I) is administered in combination with OCA. In some embodiments, the compound of Formula (II) or (I) is administered in combination with semaglutide.

In at least one embodiment, the compound of Formula (I) or (II) of the present disclosure is 2-(((5Z,8Z,11Z,14Z,17Z)-icosa-5,8,11,14,17-pentaen-1-yl)oxy)butanoic acid (Compound A):

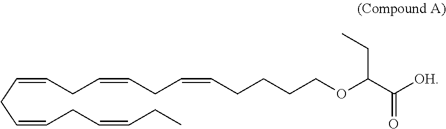 [SMILES: CC/C=C\C/C=C\C/C=C\C/C=C\C/C=C\CCCCOC(CC)C(=O)O]

In at least one embodiment, the compound of Formula (I) or (II) of the present disclosure is 2-(((5Z,8Z,11Z,14Z,17Z)-icosa-5,8,11,14,17-pentaen-1-yl)oxy)butanoic acid (Compound A) is present in its S and/or R form represented by the formulas:

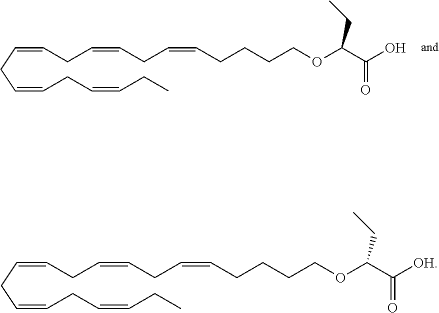 [SMILES: CC/C=C\C/C=C\C/C=C\C/C=C\C/C=C\CCCCO[C@@H](CC)C(=O)O ; CC/C=C\C/C=C\C/C=C\C/C=C\C/C=C\CCCCO[C@H](CC)C(=O)O]

In some embodiments, 2-(((5Z,8Z,11Z,14Z,17Z)-icosa-5,8,11,14, 17-pentaen-1-yl)oxy)butanoic acid (Compound A) is administered in combination with at least one additional active agent selected from firsocostat, OCA, and semaglutide.

In some embodiments, the combination therapy comprises a compound of Formula (I) or (II) and semaglutide. In some embodiments, the combination therapy comprises a compound of Formula (I) or (II) and fircosostat. In some embodiments, the combination therapy comprises a compound of Formula (I) or (II) and OCA. In some embodiments, the compound of Formula (I) or (II) is 2-(((5Z,8Z,11Z,14Z,17Z)-icosa-5,8,11,14,17-pentaen-1-yl)oxy)butanoic acid (Compound A).

In some embodiments, the use of the combination therapy decreases the level of plasma and/or liver triglycerides in the subject. In some embodiments, the use of the combination therapy decreases the level of plasma and/or liver cholesterol in the subject. In some embodiments, the use of the combination therapy reduces hepatic steatosis in the subject. In some embodiments, the use of the combination therapy reduces hepatic inflammation in the subject. In some embodiments, the use of the combination therapy reduces hepatic fibrosis in a subject. In some embodiments, the use of the combination therapy reverses steatohepatitis.

As previously described, multiple independent and interdependent metabolic, inflammatory, and ultimately fibrotic components converge in the development of human NASH. It is likely that any successful treatment will need to address multiple aspects of NASH, for example via upstream metabolic/inflammatory targets. However, as fibrosis development is associated with clinical outcomes, a NASH therapy should target the inflammatory component and also reduce or prophylactically treat the development of fibrosis. The enclosed examples show the surprising and unexpectedly potent anti-inflammatory and anti-fibrotic effects, as well as the potent effects on steatosis, of combination therapies comprising oxygen-containing structurally modified fatty acids, such as Compound A. These findings support the use of oxygen-containing compounds of the disclosure in combination therapies for use in therapeutic and/or prophylactic treatment of NASH and ASH in human subjects.

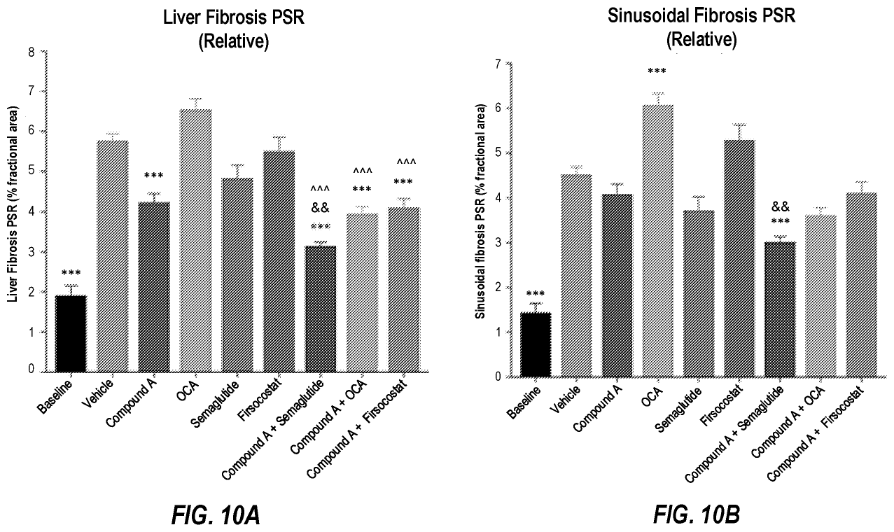

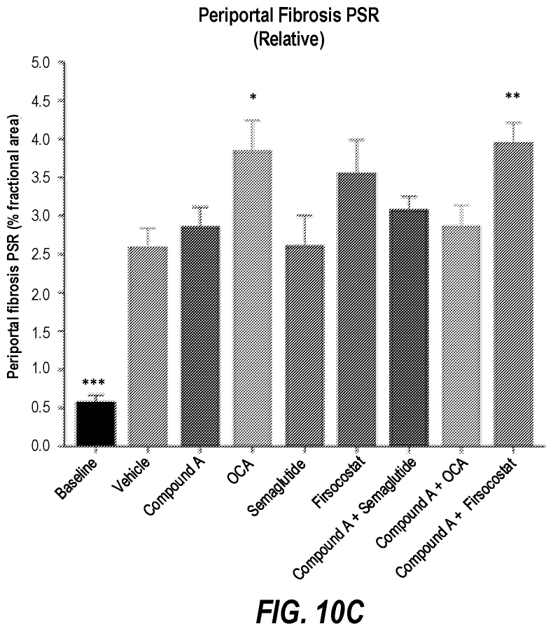

It was surprising to find that combinations therapies comprising Compound A and at least one of firsocostat, obeticholine acid (OCA), and semaglutide have greater efficacy in reducing or prophylactically treating the development of fibrosis and reversing markers of hepatic fibrosis than Compound A alone, e.g., fibrotic area as measured by histological assessment of picrosirius red staining in a CDAA-HFD (choline-deficient high-fat diet), diet-induced NASH mouse model. For example, the combination of Compound A and semaglutide significantly reduced the fibrotic area of liver, and the fibrotic sinusoidal area, as determined with picrosirius red (PSR) compared with vehicle, as well as compared with both Compound A and semaglutide alone (FIGS. 10A and 10B). The combination of Compound A and OCA significantly reduced the hepatic area expressing α-SMA compared to vehicle as well as Compound A and OCA alone (FIG. 11B). Surprisingly, this combination had a significant effect despite neither Compound A nor OCA significantly affecting α-SMA liver content compared with vehicle.

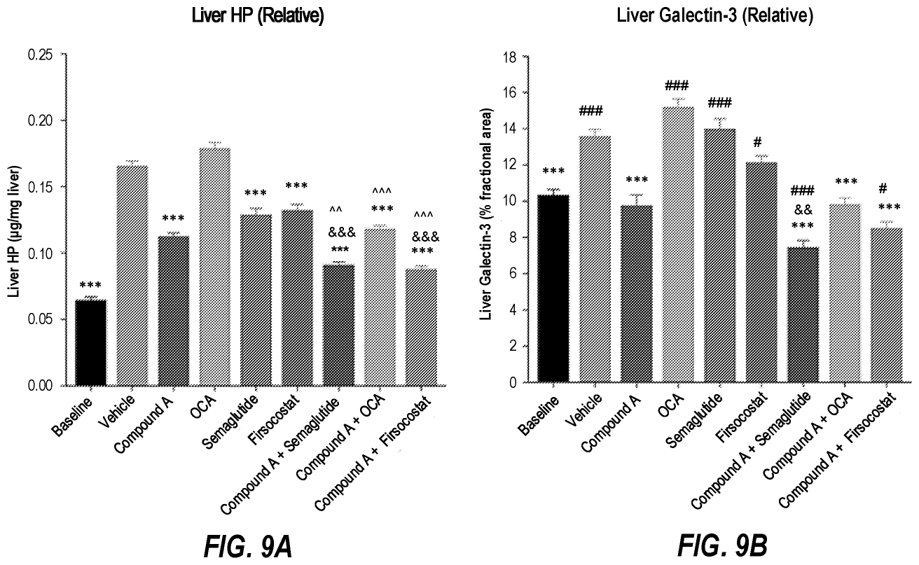

The combination of Compound A and semaglutide, and the combination of Compound A and firsocostat, unexpectedly reduced the hepatic area expressing galectin-3 below the baseline level that existed prior to initiating therapy (FIG. 9B). This was surprising in view of neither semaglutidue nor firsocostat having a significant effect on galectin-3 liver content compared with vehicle. Galectin-3 is a hepatic marker for fibrosis and inflammation.

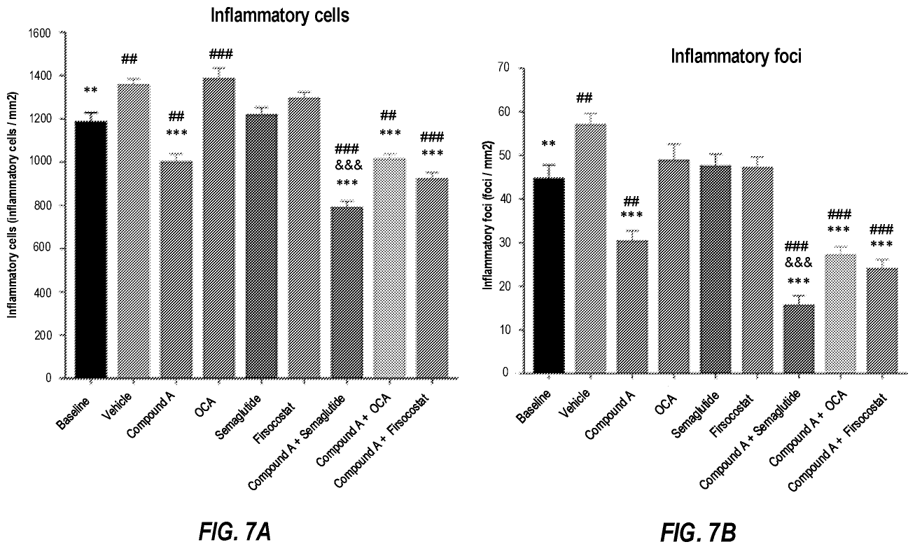

Given the importance of fibrosis in NASH-associated morbidity and mortality, the CDAA-induced NASH model results described herein support the notion that the combination therapies presented herein are effective in the treatment of NASH-related complications. This finding was also supported by the effects of the combination therapies on inflammatory responses, as well as the effects on steatosis. Surprisingly, Compound A in combination with firsocostat, Compound A in combination with semaglutide, and Compound A in combination with OCA all reduced the number of inflammatory cells and inflammatory foci to below the baseline level that existed prior to initiating therapy (FIGS. 7A and 7B). Compound A alone also reduced these indicators of inflammation below the baseline level. The combination of Compound A and semaglutide surprisingly reduced inflammatory cells and inflammatory foci significantly more than Compound A alone, despite semaglutide alone not having a significant effect compared to vehicle.

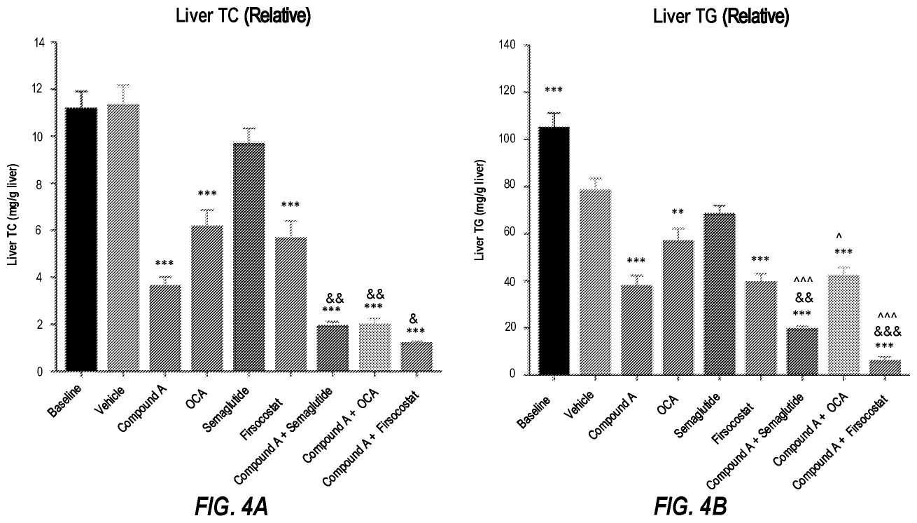

The combination therapies also significantly reduced steatosis. For example, treatment with Compound A in combination with firsocostat, Compound A in combination with semaglutide and Compound A in combination with OCA all show a greater decrease of liver triglyceride levels than compared to the groups that were treated with a monotherapy of these individual compounds (FIG. 4B). In particular, the combination of Compound A and semaglutide resulted in significantly lower levels of liver triglycerides than either agent alone, despite semaglutide alone not significantly affecting liver triglyceride levels compared to vehicle. Additionally, it was unexpected that the combination of Compound A and firsocostate greatly and significantly reduced liver triglyceride levels compared to either agent alone.

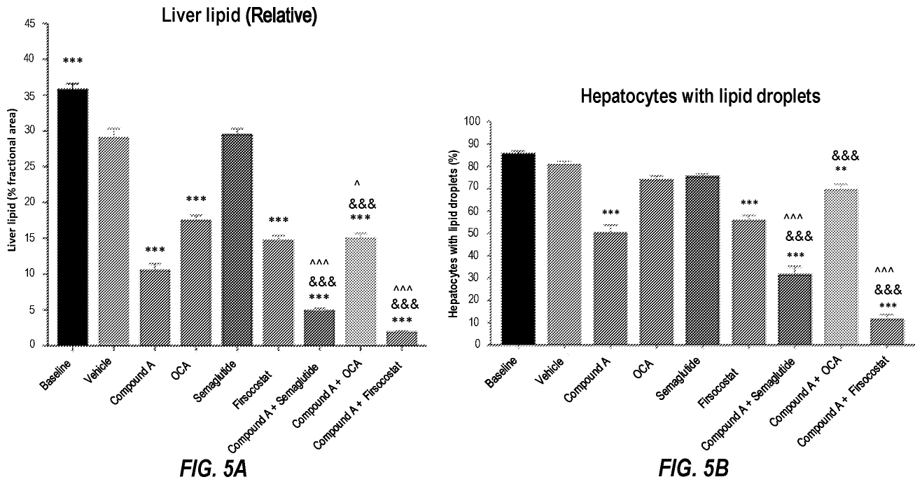

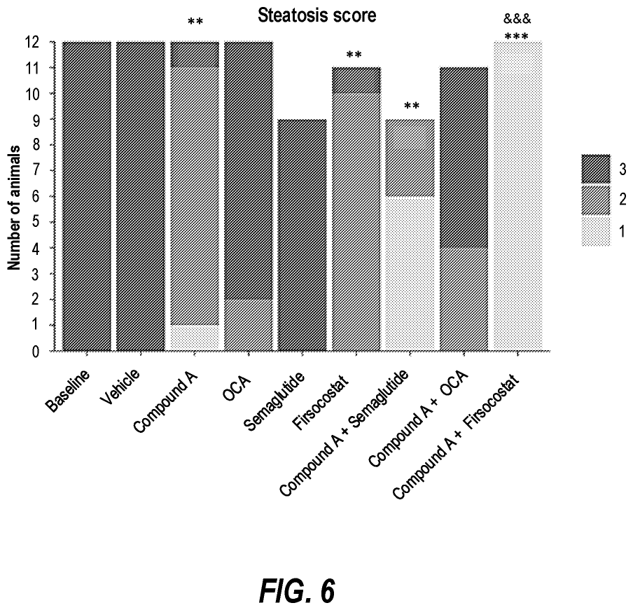

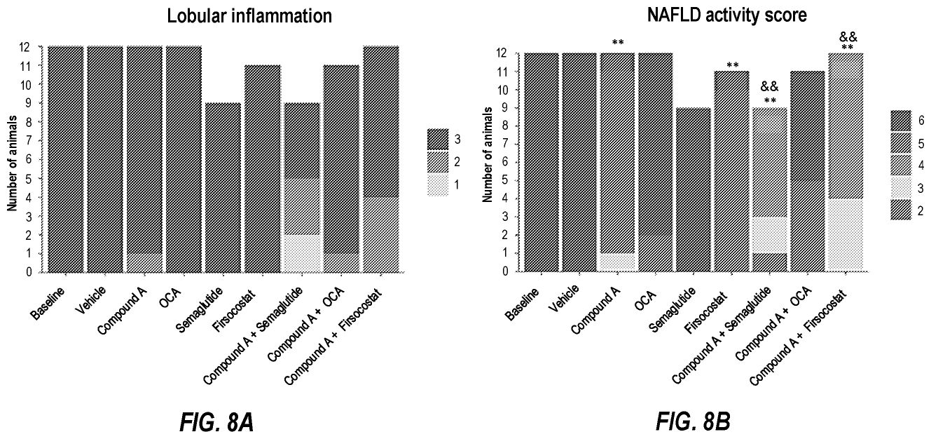

The surprising results obtained with the combinations of Compound A and semaglutide, and Compound and firsocostat, respectively, were similarly shown in the effects on the hepatic steatosis area (FIG. 5A) and the percentage of hepatocytes with lipid droplets (FIG. 5B). Specifically, the combination of Compound A and semaglutide significantly lowered hepatic steatosis area and the percentage of hepatocytes with lipid droplets more so than did Compound A alone, despite semaglutide alone not significantly affecting either parameter compared with vehicle. The combination of Compound A and firsocostat significantly lowered liver lipid content and the percentage of hepatocytes with lipid droplets to a much greater extent than either Compound A or firsocostat alone. The superior effects of these combinations are further reflected in the improvement of steatosis (FIG. 6) and NAFLD activity scores (NAS) (FIG. 8B).

The combination therapies of the present disclosure comprising compounds of Formula (II) or Formula (I) and at least one additional active agent chosen from a glucagon-like peptide 1 (GLP-1) receptor agonist, an acetyl-CoA carboxylase (ACC) inhibitor, and a farnesoid X receptor (FXR) agonist, may be administered to treat and/or reverse non-alcoholic steatohepatitis (NASH), or other hepatic disorders characterized by hepatic steatosis, fibrosis, and/or inflammation. In some embodiments, the treatment of NASH may be prophylactic. Further, the compounds may be administered to treat at least one disease, condition or risk factor associated with NASH. In some embodiments, the treatment of at least one disease, condition, or risk factor associated with NASH may be prophylactic.

In view of the similarity in the pro-inflammatory and pro-fibrotic mechanisms between NASH and alcoholic steatohepatitis (ASH), the anti-inflammatory and anti-fibrotic effects of the disclosed compounds described herein in NASH models are thus relevant for the treatment and/or reversal of ASH, in particular, the prevention of progression and induction of regression of advanced ASH and associated fibrosis.

Thus, the combination therapies comprising compounds of Formula (II), or Formula (I), and at least one additional active agent chosen from a glucagon-like peptide 1 (GLP-1) receptor agonist, an acetyl-CoA carboxylase (ACC) inhibitor, and a farnesoid X receptor (FXR) agonist may be administered to treat and/or reverse ASH. In some embodiments, the treatment of ASH may be prophylactic. Further, the compounds may be administered to treat at least one disease, condition or risk factor associated with ASH. In some embodiments, the treatment of at least one disease, condition, or risk factor associated with ASH may be prophylactic.

In some embodiments, the combination therapy comprising a compound of Formula (I) or (II) and at least one additional active agent selected from a GLP-1 receptor agonist, an ACC inhibitor, and a FXR agonist is used to decrease the level of plasma triglycerides and/or cholesterol in a subject who has NASH or ASH.

In some embodiments, the combination therapy comprising a compound of Formula (I) or (II) and at least one additional active agent selected from a GLP-1 receptor agonist, an ACC inhibitor, and a FXR agonist decreases hepatic steatosis in a subject with NASH or ASH compared with a subject with NASH or ASH who has not received therapeutic treatment. In some embodiments, the combination therapy reduces relative hepatic triglyceride and/or cholesterol levels in a subject who has NASH or ASH compared with a subject with NASH or ASH who has not received therapeutic treatment. In some embodiments, the combination therapy reduces the hepatic steatosis area of liver lipids in a subject who has NASH or ASH compared with a subject with NASH or ASH who has not received therapeutic treatment. In some embodiments, the combination therapy reduces the percentage of hepatocytes with lipid droplets in a subject who has NASH or ASH compared with a subject with NASH or ASH who has not received therapeutic treatment. In some embodiments, the combination therapy reduces the steatosis score in a subject who has NASH or ASH compared with a subject who has NASH or ASH who has not received therapeutic treatment. In some embodiments the combination therapy comprises a compound of Formula (I) or (II) and semaglutide. In some embodiments the combination therapy comprises a compound of Formula (I) or (II) and firsocostat. In some embodiments, the combination therapy comprises a compound of Formula (I) or (II) and OCA. In some embodiments, the compound of Formula (I) or (II) is Compound A.

In some embodiments, the combination therapy comprising a compound of Formula (I) or (II) and at least one additional active agent selected from a GLP-1 receptor agonist, an ACC inhibitor, and a FXR agonist decreases hepatic steatosis in a subject with fatty liver disease compared with a subject fatty liver disease who has not received therapeutic treatment. In some embodiments, the combination therapy reduces relative hepatic triglyceride and/or cholesterol levels in a subject who has fatty liver disease compared with a subject with fatty liver disease who has not received therapeutic treatment. In some embodiments, the combination therapy reduces the hepatic steatosis area of liver lipids in a subject who has fatty liver disease compared with a subject with fatty liver disease who has not received therapeutic treatment. In some embodiments, the combination therapy reduces the percentage of hepatocytes with lipid droplets in a subject who has fatty liver disease compared with a subject with NASH or ASH who has not received therapeutic treatment. In some embodiments, the combination therapy reduces the steatosis score in a subject who has fatty liver disease compared with a subject who has fatty liver disease who has not received therapeutic treatment. In some embodiments the combination therapy comprises a compound of Formula (I) or (II) and semaglutide. In some embodiments the combination therapy comprises a compound of Formula (I) or (II) and firsocostat. In some embodiments, the combination therapy comprises a compound of Formula (I) or (II) and OCA. In some embodiments, the compound of Formula (I) or (II) is Compound A. In some embodiments, the fatty liver disease is non-alcoholic fatty liver disease (NAFLD). In some embodiments, the fatty liver disease is alcoholic fatty liver disease (ALFD). In some embodiments, the fatty liver disease is not accompanied by an inflammatory response and cellular damage.

The steatosis score of a subject is determined by criteria outlined in Kleiner et al., Hepatology, 2005; 41, as a component of the NAFLD activity score (NAS) and as described in the biological examples herein. Specifically, and as shown in Table 3 of the examples, NAS is a composite score assessing steatosis as determined by the percentage of hepatocytes with lipid droplets, lobular inflammation as determined by the number of inflammatory foci, and quantitative assessment of ballooning degeneration. Fatty liver disease that is not accompanied by an inflammatory response and cellular damage has neither lobular inflammation nor ballooning degeneration as evaluated under the NAS assessment. The hepatic steatosis area is determined as described in the biological examples.

In some embodiments, the combination therapy comprising a compound of Formula (I) or (II) and at least one additional active agent selected from a GLP-1 receptor agonist, an ACC inhibitor, and a FXR agonist decreases hepatic inflammation in a subject with NASH or ASH compared with a subject with NASH or ASH who has not received therapeutic treatment. In some embodiments, the combination therapy decreases the number of hepatic inflammatory cells and/or the number of hepatic inflammatory foci in a subject with NASH or ASH compared with a subject with NASH or ASH who has not received therapeutic treatment. In some embodiments the combination therapy comprises a compound of Formula (I) or (II) and semaglutide. In some embodiments the combination therapy comprises a compound of Formula (I) or (II) and firsocostat. In some embodiments, the combination therapy comprises a compound of Formula (I) or (II) and OCA. In some embodiments, the compound of Formula (I) or (II) is Compound A.

In some embodiments, the combination therapy comprising a compound of Formula (I) or (II) and at least one additional active agent selected from a GLP-1 receptor agonist, an ACC inhibitor, and a FXR agonist decreases the NAFLD activity score (NAS) in a subject with NASH or ASH compared with a subject with NASH or ASH who has not received therapeutic treatment. In some embodiments the combination therapy comprises a compound of Formula (I) or (II) and semaglutide. In some embodiments the combination therapy comprises a compound of Formula (I) or (II) and firsocostat. In some embodiments, the combination therapy comprises a compound of Formula (I) or (II) and OCA. In some embodiments, the compound of Formula (I) or (II) is Compound A.

The NAFLD activity score (NAS) of a subject is determined as outlined in Kleiner et al., Hepatology, 2005; 41 and as described in the biological examples herein.

In some embodiments, the combination therapy comprising a compound of Formula (I) or (II) and at least one additional active agent selected from a GLP-1 receptor agonist, an ACC inhibitor, and a FXR agonist decreases hepatic fibrosis in a subject with NASH or ASH compared with a subject with NASH or ASH who has not received therapeutic treatment. In some embodiments, the combination therapy reduces the level of relative liver galectin-3 in a subject with NASH or ASH compared with a subject with NASH or ASH who has not received therapeutic treatment. In some embodiments, the combination therapy reduces the liver hydroxyproline content in a subject. In some embodiments, the combination therapy reduces the hepatic fibrotic area determined by PSR in a subject with NASH or ASH compared with a subject with NASH or ASH who has not received therapeutic treatment. In some embodiments, the combination therapy reduces the hepatic sinusoidal fibrotic area determined by PSR in a subject with NASH or ASH compared with a subject with NASH or ASH who has not received therapeutic treatment. In some embodiments, the combination therapy reduces the hepatic area expressing α-smooth muscle actin in a subject with NASH or ASH compared with a subject with NASH or ASH who has not received therapeutic treatment. In some embodiments the combination therapy comprises a compound of Formula (I) or (II) and semaglutide. In some embodiments the combination therapy comprises a compound of Formula (I) or (II) and firsocostat. In some embodiments, the combination therapy comprises a compound of Formula (I) or (II) and OCA. In some embodiments, the compound of Formula (I) or (II) is Compound A.

In some embodiments, the combination therapy comprising a compound of Formula (II) and at least one additional active agent selected from GLP-1 receptor agonist, an ACC inhibitor, and a FXR agonist, further comprises a third or more additional active agent(s) independently chosen from angiotensin II receptor antagonists, angiotensin converting enzyme (ACE) inhibitors, apoptosis signal-regulating kinase-1 (ASK1) inhibitors, caspase inhibitors, cathepsin B inhibitors, CCR2 chemokine antagonists, CCR5 chemokine antagonists, chloride channel stimulators, cholesterol solubilizers, diacyl glycerol O-acyltransferase 1 (DGAT1) inhibitors, dipeptidyl peptidase IV (DPP IV) inhibitors, fibroblast-growth factor (FGF)-21 agonists, anti-CD3 mAb, galectin-3 inhibitors, glutathione precursors, hepatitis C virus NS3 protease inhibitors, HMG CoA reductase inhibitors, 1 Iβ-hydroxysteroid dehydrogenase (I Iβ-HSDI) inhibitors, heat shock protein (Hsp)47 inhibitors, IL-Iβ antagonists, IL-6 antagonists, IL-10 agonists, IL-17 antagonists, ileal sodium bile acid co-transporter inhibitors, leptin analogs, 5-lipoxygenase inhibitors, LPL gene stimulators, lysyl oxidase homolog 2 (LOXL2) inhibitors, lysophosphatidic acid 1 (LPA1) receptor antagonists, omega-3 fatty acids, PDE3 inhibitors, PDE4 inhibitors, phospholipase C (PLC) inhibitors, PPARa agonists, PPARy agonists, PPAR5 agonists, recombinant human pentraxin-2 protein (PRF-1), Rho associated protein kinase 2 (ROCK2) inhibitors, semicarbazide-sensitive amine oxidase (SSAO) inhibitors, sodium glucose transporter-2 (SGLT2) inhibitors, stearoyl CoA desaturase-1 inhibitors, thyroid hormone receptor β agonists, tumor necrosis factor α (TNFα) ligand inhibitors, transglutaminase inhibitors, transglutaminase inhibitor precursors and small activating RNA (saRNA).

In some embodiment, there is provided a method of treating non-alcoholic steatohepatitis and/or alcoholic steatohepatitis in a subject in need thereof, the method comprising administering to the subject a combination therapy comprising a compound of Formula (II) and at least one additional active agent selected from a glucagon-like peptide 1 (GLP-1) receptor agonist, an acetyl-CoA carboxylase (ACC) inhibitor, and a farnesoid X receptor (FXR) agonist 1. In some embodiments the combination therapy comprises a compound of Formula (I) or (II) and semaglutide. In some embodiments the combination therapy comprises a compound of Formula (I) or (II) and firsocostat. In some embodiments, the combination therapy comprises a compound of Formula (I) or (II) and OCA. In some embodiments, the compound of Formula (I) or (II) is Compound A.

In some embodiments, administration of the combination therapy in the methods of the present disclosure is by simultaneous administration. In some embodiments, administration of the combination therapy in the methods of the present disclosure is by sequential administration. In some embodiments, administration of the combination therapy in the methods of the present disclosure is by overlapping administration. In some embodiments, administration of the combination therapy in the methods of the present disclosure is by interval administration. In some embodiments, administration of the combination therapy in the methods of the present disclosure is by continuous administration.

The present disclosure is also directed to use of the described combination therapies in the therapeutic and/or prophylactic treatment of NASH and/or ASH. The present disclosure is likewise directed to use of the described combination therapies in the manufacture of a medicament for the therapeutic and/or prophylactic treatment of NASH and/or ASH.

In some embodiments, a combination therapy of the present disclosure reduces plasma triglyceride levels by about 20%, 25%, 30%, 35%, or 40% in a subject with NASH or ASH as compared to a subject with NASH or ASH who does not receive treatment. In some embodiments, a combination therapy of the present disclosure reduces plasma triglyceride levels by 20-30%, 30-40%, or 10-40%. In some embodiments, a combination therapy of the present disclosure reduces plasma total cholesterol levels by by about 10%, 20%, 30%, 40%, 50%, 60%, 70%, or 80% in a subject with NASH or ASH as compared to a subject with NASH or ASH who does not receive treatment. In some embodiments, a combination therapy of the present disclosure reduces plasma total cholesterol levels by 20-30%, 20-25%, 25-30%, 30-40%, 30-35%, 35-40%, 40-50%, 40-45%, 45-50%, 50-60%, 50-55%, 55-60%, 60-70%, 60-65%, 65-70%, 70-80%, 70-75%, or 75-80%.

In some embodiments, a combination therapy of the present disclosure reduces liver total cholesterol levels by about 20%, 30%, 40%, 50%, 60%, 70%, 80%, or 90% in a subject with NASH or ASH as compared to a subject with NASH or ASH who does not receive treatment. In some embodiments, a combination therapy of the present disclosure reduces liver total cholesterol levels by about 20%, 30%, 40%, 50%, 60%, 70%, 80%, or 90% in a subject with NAFLD or ALD as compared to a subject with NAFLD or ALD who does not receive treatment. In some embodiments, both subjects have fatty liver disease that is not accompanied by an inflammatory response and cellular damage. In some embodiments, a combination therapy of the present disclosure reduces liver total cholesterol levels by 20-30%, 20-25%, 25-30%, 30-40%, 30-35%, 35-40%, 40-50%, 40-45%, 45-50%, 50-60%, 50-55%, 55-60%, 60-70%, 60-65%, 65-70%, 70-80%, 70-75%, 75-80%, 80-90%, 80-85%, or 85-90%.

In some embodiments, a combination therapy of the present disclosure reduces liver triglyceride levels by about 20%, 30%, 40%, 50%, 60%, 70%, 80%, or 90% in a subject with NASH or ASH as compared to a subject with NASH or ASH who does not receive treatment. In some embodiments, a combination therapy of the present disclosure reduces liver triglyceride levels by about 20%, 30%, 40%, 50%, 60%, 70%, 80%, or 90% in a subject with NAFLD or ALD as compared to a subject with NAFLD or ALD who does not receive treatment. In some embodiments, both subjects have fatty liver disease that is not accompanied by an inflammatory response and cellular damage. In some embodiments, a combination therapy of the present disclosure reduces liver triglyceride levels by 20-30%, 20-25%, 25-30%, 30-40%, 30-35%, 35-40%, 40-50%, 40-45%, 45-50%, 50-60%, 50-55%, 55-60%, 60-70%, 60-65%, 65-70%, 70-80%, 70-90%, 70-75%, 75-80%, 80-90%, 80-85%, or 85-90%.

In some embodiments, a combination therapy of the present disclosure reduces hepatic steatosis area by about 20%, 30%, 40%, 50%, 60%, 70%, 80%, or 90% in a subject with NASH or ASH as compared to a subject with NASH or ASH who does not receive treatment. In some embodiments, a combination therapy of the present disclosure reduces hepatic steatosis area by about 20%, 30%, 40%, 50%, 60%, 70%, 80%, or 90% in a subject with NAFLD or ALD disease as compared to a subject with NAFLD or ALD who does not receive treatment. In some embodiments, both subjects have fatty liver disease that is not accompanied by an inflammatory response and cellular damage. In some embodiments, a combination therapy of the present disclosure reduces hepatic steatosis area by 20-30%, 20-25%, 25-30%, 30-40%, 30-35%, 35-40%, 40-50%, 40-45%, 45-50%, 50-60%, 50-55%, 55-60%, 60-70%, 60-65%, 65-70%, 70-80%, 70%-90%, 70-75%, 75-80%, 80-90%, 80-85%, or 85-90%.

In some embodiments, a combination therapy of the present disclosure reduces the percentage of hepatocytes with lipid droplets by about 20%, 30%, 40%, 50%, 60%, 70%, 80%, or 90% in a subject with NASH or ASH as compared to a subject with NASH or ASH who does not receive treatment. In some embodiments, a combination therapy of the present disclosure reduces the percentage of hepatocytes with lipid droplets by about 20%, 30%, 40%, 50%, 60%, 70%, 80%, or 90% in a subject with NAFLD or ALD as compared to a subject with NAFLD or ALD who does not receive treatment. In some embodiments, both subjects have fatty liver disease that is not accompanied by an inflammatory response and cellular damage. In some embodiments, a combination therapy of the present disclosure reduces the percentage of hepatocytes with lipid droplets by 20-30%, 20-25%, 25-30%, 30-40%, 30-35%, 35-40%, 40-50%, 40-45%, 45-50%, 50-60%, 50-55%, 55-60%, 60-70%, 60-65%, 65-70%, 70-80%, 70-90%, 70-75%, 75-80%, 80-90%, 80-85%, or 85-90%.

In some embodiments, a combination therapy of the present disclosure reduces inflammatory cells by about 10%, 20%, 30%, 40%, or 45% in a subject with NASH or ASH as compared to a subject with NASH or ASH who does not receive treatment. In some embodiments, a combination therapy of the present disclosure reduces inflammatory cells by 10-20%, 10-15%, 15-20%, 20-30%, 20-25%, 25-30%, 30-40%, 30-50%, 30-35%, 35-40%, 40-50%, or 40-45%. In some embodiments, a combination therapy of the present disclosure reduces inflammatory foci by about 20%, 30%, 40%, 50%, 60%, 70%, or 75% in a subject with NASH or ASH as compared to a subject with NASH or ASH who does not receive treatment. In some embodiments, a combination therapy of the present disclosure reduces inflammatory foci by 20-30%, 20-25%, 25-30%, 30-40%, 30-35%, 35-40%, 40-50%, 40-45%, 45-50%, 50-60%, 50-55%, 55-60%, 60-70%, 60-80%, 60-65%, 65-70%, 70-80%, or 70-75%.

In some embodiments, a combination therapy of the present disclosure reduces hepatic area expressing galectin-3 by about 20%, 30%, 40%, or 50% in a subject with NASH or ASH as compared to a subject with NASH or ASH who does not receive treatment. In some embodiments, a combination therapy of the present disclosure reduces hepatic area expressing galectin-3 by 20-30%, 20-25%, 25-30%, 30-40%, 30-35%, 35-40%, 40-50%, 40-45%, or 45-50%.

In some embodiments, a combination therapy of the present disclosure reduces liver hydroxyproline levels by about 20%, 30%, or 40% in a subject with NASH or ASH as compared to a subject with NASH or ASH who does not receive treatment. In some embodiments, a combination therapy of the present disclosure reduces liver hydroxyproline levels by 20-30%, 20-25%, 25-30%, 20-40%, 30-40%, 30-35%, or 35-40%.

In some embodiments, a combination therapy of the present disclosure reduces hepatic fibrotic area determined by PSR by about 20%, 30%, 40%, or 50% in a subject with NASH or ASH as compared to a subject with NASH or ASH who does not receive treatment. In some embodiments, a combination therapy of the present disclosure reduces hepatic fibrotic area determined by PSR 20-30%, 20-25%, 25-30%, 30-40%, 30-50%, 30-35%, 35-40%, 40-50%, 40-45%, or 45-50%. In some embodiments, a combination therapy of the present disclosure reduces hepatic sinusoidal fibrotic area determined by PSR by about 20%, 30%, or 40%. In some embodiments, a combination therapy of the present disclosure reduces hepatic fibrotic sinusoidal fibrotic area by PSR 20-30%, 20-25%, 25-30%, 30-40%, 30-35%, or 35-40%.

In some embodiments, a combination therapy of the present disclosure reduces hepatic area expressing α-SMA by about 5%, 10%, 20%, or 30% in a subject with NASH or ASH as compared to a subject with NASH or ASH who does not receive treatment. In some embodiments, a combination therapy of the present disclosure reduces hepatic area expressing α-SMA by 5-10%, 10-20%, 10-15%, 15-20%, 20-30%, 20-25%, or 25-30%.

Some embodiments relate to a compound of Formula (II) as a monotherapy for use in reversing heaptic steatosis in a subject with NAFLD or ALD. In some embodiments, a compound of Formula (II) reverses hepatic steatosis area in a subject with NAFLD or ALD. In some embodiments, the fatty liver disease is not not accompanied by an inflammatory response and cellular damage.

Some embodiments relate to a compound of Formula (II) as a monotherapy for use in reversing hepatic steatosis and inflammation in a subject with NASH or ASH. In some embodiments, a compound of Formula (II) reverses hepatic steatosis area in a subject with NASH or ASH. In some embodiments, a compound of Formula (II) reverses hepatic inflammation in a subject with NASH or ASH.

### Compounds of Formula (I) and (II)

Compounds of Formula (I) and Formula (II) can be prepared as described, for example, in PCT Applications WO2009/061208, WO2010/128401, WO2011/089529, WO2016/156912, WO2019/111048, and according to Examples below. In addition, Compound A can be prepared as described, for example, in PCT Applications WO2010/128401, WO2014/132135, and WO2019/111048 and according to Example 2 below. These publications are incorporated herein by reference.

The Examples provided below are exemplary and one skilled in the art would understand how to apply these general methods to arrive at other compounds within the scope of Formula (I) and Formula (II). Compounds of the present disclosure may be in the form of a pharmaceutically acceptable salt or ester. For example, the compounds of Formula (I) and Formula (II) may be in the form of esters, such as a phospholipid, a glyceride or a C₁-C₆-alkyl ester. In at least one embodiment, the ester is chosen from a glyceride or a C₁-C₆-alkyl ester. In at least one embodiment, the ester is chosen from a triglyceride, a 1,2-diglyceride, a 1,3-diglyceride, a 1-monoglyceride, a 2-monoglyceride, a methyl ester, an ethyl ester, a propyl ester, an isopropyl ester, an n-butyl ester and a tert-butyl ester. In at least one embodiment, the compound of Formula (I) is present as a methyl ester, an ethyl ester, an isopropyl ester, a n-butyl ester or a tert-butyl ester, for example as a methyl ester or an ethyl ester. Typically, esters represented by Formula (I) (e.g., ethyl esters) will be hydrolyzed in the gastrointestinal tract.

Salts suitable for the present disclosure include, but are not limited to, salts of NH₄⁺; metal ions such as Li⁺, Na⁺, K⁺, Mg²⁺, or Ca²⁺; a protonated primary amine such as tertbutyl ammonium, (3S,5S,7S)-adamantan-1-ammonium, 1,3-dihydroxy-2-(hydroxymethyl)propan-2-ammonium, a protonated aminopyridine (e.g., pyridine-2-ammonium); a protonated secondary amine such as diethylammonium, 2,3,4,5,6-pentahydroxy-N-methylhexan-1-ammonium, N-ethylnaphthalen-1-ammonium, a protonated tertiary amine such as 4-methylmorpholin-4-ium, a protonated quaternary amine such as 2-hydroxy-N,N, N-trimethylethan-1-aminium and a protonated guanidine such as amino((4-amino-4-carboxybutyl)amino)methaniminium or a protonated heterocycle such as 1H-imidazol-3-ium. Additional examples of suitable salts include salts of a diprotonated diamine such as ethane-1,2-diammonium or piperazine-1,4-diium. Other salts according to the present disclosure may comprise protonated Chitosan:

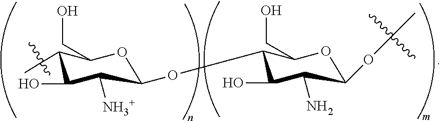

In at least embodiment, the salts are chosen from a sodium salt, a calcium salt, and a choline salt. In one embodiment the salt is a sodium salt or a calcium salt.

The present disclosure provides for a method of treating NASH or ASH in a subject in need thereof, comprising co-administering to the subject a pharmaceutically effective amount of a compound of Formula (I) or Formula (II) and at least one additional active agent chosen from a glucagon-like peptide 1 (GLP-1) receptor agonist, an acetyl-CoA carboxylase (ACC) inhibitor, and a farnesoid X receptor (FXR) agonist. The subject may be a human or a non-human mammal. The compounds presently disclosed may be co-administered as a medicament, such as in a pharmaceutical composition. One embodiment provides for a pharmaceutical composition comprising a compound of Formula (II) or Formula (I), such as Compound A and at least one additional active agent chosen from a glucagon-like peptide 1 (GLP-1) receptor agonist, an acetyl-CoA carboxylase (ACC) inhibitor, and a farnesoid X receptor (FXR) agonist, for use in treating non-alcoholic steatohepatitis. The composition presently disclosed may optionally further comprise at least one non-active pharmaceutical ingredient, i.e., excipient. Non-active ingredients may solubilize, suspend, thicken, dilute, emulsify, stabilize, preserve, protect, color, flavor, and/or fashion active ingredients into an applicable and efficacious preparation, such that it may be safe, convenient, and/or otherwise acceptable for use. Examples of excipients include, but are not limited to, solvents, carriers, diluents, binders, fillers, sweeteners, aromas, pH modifiers, viscosity modifiers, antioxidants, extenders, humectants, disintegrating agents, solution-retarding agents, absorption accelerators, wetting agents, absorbents, lubricants, coloring agents, dispersing agents, and preservatives. Excipients may have more than one role or function or may be classified in more than one group; classifications are descriptive only and are not intended to be limiting. In some embodiments, for example, the at least one excipient may be chosen from corn starch, lactose, glucose, microcrystalline cellulose, magnesium stearate, polyvinylpyrrolidone, citric acid, tartaric acid, water, ethanol, glycerol, sorbitol, polyethylene glycol, propylene glycol, cetylstearyl alcohol, carboxymethylcellulose, and fatty substances such as hard fat or suitable mixtures thereof. In some embodiments, the compositions presently disclosed further comprise at least one pharmaceutically acceptable antioxidant, e.g., tocopherol such as alpha-tocopherol, beta-tocopherol, gamma-tocopherol, and delta-tocopherol, or mixtures thereof, BHA such as 2-tert-butyl-4-hydroxyanisole and 3-tert-butyl-4-hydroxyanisole, or mixtures thereof and BHT (3,5-di-tert-butyl-4-hydroxytoluene), or mixtures thereof.

The compounds of Formula (I) and (II) presently disclosed may be formulated in one or more oral administration forms, e.g., tablets or gelatin soft or hard capsules. The dosage forms can be of any shape suitable for oral administration, such as spherical, oval, ellipsoidal, cube-shaped, regular, and/or irregular shaped. Conventional formulation techniques known in the art may be used to formulate the compounds according to the present disclosure. In some embodiments, the composition may be in the form of a gelatin capsule or a tablet.

The first component of the combined product, i.e., the compound of Formula (I) or (II) may be administered or formulated in any manner as described above. The second component of the combined product, the additional active agent, may be formulated as is suitable for the type of agent it is, and depends on several factors, including the mode of administration of the agent.

In some embodiments, the co-administration of the combination therapy is by simultaneous administration. In some embodiments, the co-administration is by sequential administration. In some embodiments, the co-administration is by overlapping administration. In some embodiments, the co-administration is by interval administration. In some embodiments, the co-administration is by continuous administration.

### Dosages

The present disclosure relates to combination therapies for use in treating NASH and/or ASH that comprise at least one compound of Formula (I) or (II) and at least one additional active agent. In some embodiments, the at least one additional active agent is chosen from a GLP-1 receptor agonist, an ACC inhibitor, and a FXR agonist.

A suitable daily dosage of a compound of Formula (I) or a compound of Formula (II) may range from about 5 mg to about 4 g, such as from about 5 mg to about 2 g. For example, in some embodiments, the daily dose ranges from about 10 mg to about 1.5 g, from about 50 mg to about 1 g, from about 100 mg to about 1 g, from about 150 mg to about 900 mg, from about 50 mg to about 800 mg, from about 100 mg to about 800 mg, from about 100 mg to about 600 mg, from about 150 to about 550 mg, or from about 200 to about 500 mg. In at least one embodiment, the daily dose ranges from about 200 mg to about 600 mg. In at least one embodiment, the daily dose is about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, or about 900 mg. The compound(s) may be administered, for example, once, twice, or three times per day.

In at least one embodiment, the compound of Formula (I) is administered in an amount ranging from about 200 mg to about 800 mg per dose. In at least one embodiment, the compounds are administered once per day. In at least one embodiment, the compounds are administered once per day at a dose of 750 mg. In some embodiments, compounds are administered once per day at a dose of 600 mg. In some embodiments, compounds are administered once per day at a dose of 500 mg. In some embodiments, compounds are administered once per day at a dose of 300 mg. In some embodiments, compounds are administered once per day at a dose of 250 mg. In some embodiments, compounds are administered once per day at a dose of 300 mg or 600 mg.

In at least one embodiment, the compound of Formula (II) is administered in an amount ranging from about 200 mg to about 800 mg per dose. In at least one embodiment, the compounds are administered once per day. In at least one embodiment, the compounds are administered once per day at a dose of 750 mg. In some embodiments, the compounds are administered once per day at a dose of 600 mg. In some embodiments, the compounds are administered once per day at a dose of 500 mg. In some embodiments the compounds are administered once per day at a dose of 300 mg. In some embodiments the compounds are administered once per day at a dose of 250 mg. In some embodiments, the compounds are administered once per day at a dose of 300 mg or 600 mg.

In some embodiments, the at least one additional active agent of the disclosed combination therapies is a GLP-1 receptor agonist. GLP-1 receptor agonists, or incretin mimetics, are agonists of the Glucagon-like peptide-1 receptor. This class of drugs is typically used for the treatment of type 2 diabetes. A non-limiting example list of GLP-agonists includes: exenatide, liraglutide, lixisenatide, albiglutide, dulaglutide, taspoglutide and semaglutide. In preferred embodiments, the at least one additional active agent of the combination therapy is semaglutide.

The human equivalent dose can be calculated from the doses used in pre-clinical mouse models by using a mouse to human multiple of 12.3 (Nair et al., J Basic Clin Pharm, 2016, 7:27-31).

In at some embodiments, the at least one additional agent of the combination therapy is semaglutide. In some embodiments, the daily dose of semaglutide ranges from about 50 μg to about 500 μg, from about 75 μg to about 250 μg, from about 75 μg to about 150 μg, from about 100 μg to about 150 μg, from about 0.1 mg to about 10 mg, from about 0.2 mg to about 8 mg, from about 0.5 mg to about 7 mg, or from about 1 mg to about 5 mg. In some embodiments, semaglutide is administered at a daily dose of about 0.1 mg to about 0.2 mg. In some embodiments, semaglutide is administered a daily dose of about 75 μg to about 150 μg. In some embodiments, semaglutide is administered at a daily dose of about 75 μg to about 125 μg. Semaglutide may be administered, for example, once, twice, or three times per day. In some embodiments, semaglutide is administered once per day.

In some embodiments, the daily dose of semaglutide is about 50 μg, about 75 μg, about 100 μg, about 125 μg, about 150 μg, about 0.2 mg, about 0.3 mg, about 0.4 mg, about 0.5 mg, about 0.6 mg, about 0.7 mg, about 0.8 mg, about 0.9 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, or about 10 mg. In some embodiments, semaglutide is administered once per day at a dose of 0.5 mg, 1 mg, 2 mg, 3 mg, 4 mg, 5 mg, 6 mg, 7 mg, 8 mg, 9 mg, or 10 mg.

In some embodiments, the at least one additional active agent of the disclosed combination therapies is an ACC inhibitor. In preferred embodiments, the ACC inhibitor is firsocostat.

In some embodiments, the at least one additional agent of the combination therapy is firsocostat. In some embodiments, the daily dose of firsocostat ranges from about 5 mg to about 3 g, from about 50 mg to about 2.5 g, from about 100 mg to about 2 g, from about 500 mg to about 3 g, from about 1 g to about 2.5 g, from about 1.5 g to about 2.5 g, from about 5 mg to about 500 mg, from about 10 mg to about 300 mg, from about 20 mg to about 200 mg, or from about 50 mg to 100 mg. In some embodiments the daily dose of firsocostat is about 10 mg/kg to about 50 mg/kg. In some embodiments, the daily dose of firsocostat is about 15 mg/kg to about 40 mg/kg. In some embodiments, the daily dose of firsocostat is about 20 mg/kg to about 30 mg/kg. Firsocostat may be administered, for example, once, twice, or three times per day. In some embodiments, firsocostat is administered once per day.

In some embodiments, the daily dose of firsocostat is about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 225 mg, about 250 mg, about 275 mg, about 300 mg, about 350 mg, about 400 mg, about 450 mg, or about 500 mg. In some embodiments, the daily dose of firsocostat is about 1 g, about 1.5 g, about 2 g, or about 2.5 g. In some embodiments, firsocostat is administered once per day at a dose of about 15 mg/kg, about 20 mg/kg, about 25 mg/k, about 30 mg/kg, about 500 mg, about 1.5 g, about 2 g, or about 2.5 g.

In some embodiments, the at least one additional active agent of the disclosed combination therapies is a FXR inhibitor. In preferred embodiments, the FXR inhibitor is obeticholic acid.

In some embodiments, the at least one additional agent of the combination therapy is OCA. In some embodiments, the daily dose of OCA ranges ranges from about 0.5 mg to about 250 mg, from about 2 mg to about 250 mg, from about 5 mg to about 200 mg, from about 50 mg to about 200 mg, from about 100 mg to about 200 mg, from about 150 mg to about 200 mg, or from about 5 mg to 20 mg. In some embodiments, OCA is administered in a daily dose of about 170 mg. OCA may be administered, for example, once, twice, or three times per day. In some embodiments, OCA is administered once per day.

In some embodiments, the daily dose of OCA is about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 12 mg, about 13 mg, about 14 mg, about 15 mg, about 16 mg, about 17 mg, about 18 mg, about 19 mg, about 20 mg. In some embodiments, OCA is administered once per day at a dose of 2.5 mg, 5 mg, 7.5 mg, 10 mg, or 15 mg. In some embodiments, OCA is administered at a daily dose of about 50 mg, about 60 mg, about 70 mg, about 90 mg, about 100 mg, about 110 mg, about 120 mg, about 130 mg, about 140 mg, about 150 mg, about 160 mg, about 170 mg, about 180 mg, about 190 mg, or about 200 mg. In some embodiments, OCA is administered once per day.

### Additional Combinations

According to the present disclosure, the combination therapies comprising at least one compound of Formula (I) or (II) and at least a second active agent selected from a GLP-1 receptor agonist, an ACC inhibitor, and a FXR agonist may be co-administered with a third or more further additional active agent. In some embodiments, the GLP-1 receptor agonist is semaglutide, the ACC inhibitor is firsocostat, and the FXR agonist is OCA.

In some embodiments, the third or more active agent is chosen from angiotensin II receptor antagonists, angiotensin converting enzyme (ACE) inhibitors, apoptosis signal-regulating kinase-1 (ASK1) inhibitors, caspase inhibitors, cathepsin B inhibitors, CCR2 chemokine antagonists, CCR5 chemokine antagonists, chloride channel stimulators, cholesterol solubilizers, diacyl glycerol O-acyltransferase 1 (DGAT1) inhibitors, dipeptidyl peptidase IV (DPP IV) inhibitors, fibroblast-growth factor (FGF)-21 agonists, anti-CD3 mAb, galectin-3 inhibitors, glutathione precursors, hepatitis C virus NS3 protease inhibitors, HMG CoA reductase inhibitors, 1 Iβ-hydroxysteroid dehydrogenase (I Iβ-HSDI) inhibitors, heat shock protein (Hsp)47 inhibitors, IL-Iβ antagonists, IL-6 antagonists, IL-10 agonists, IL-17 antagonists, ileal sodium bile acid co-transporter inhibitors, leptin analogs, 5-lipoxygenase inhibitors, LPL gene stimulators, lysyl oxidase homolog 2 (LOXL2) inhibitors, lysophosphatidic acid 1 (LPA1) receptor antagonists, omega-3 fatty acids, PDE3 inhibitors, PDE4 inhibitors, phospholipase C (PLC) inhibitors, PPARa agonists, PPARy agonists, PPAR5 agonists, recombinant human pentraxin-2 protein (PRF-1), Rho associated protein kinase 2 (ROCK2) inhibitors, semicarbazide-sensitive amine oxidase (SSAO) inhibitors, sodium glucose transporter-2 (SGLT2) inhibitors, stearoyl CoA desaturase-1 inhibitors, thyroid hormone receptor β agonists, tumor necrosis factor α (TNFα) ligand inhibitors, transglutaminase inhibitors, transglutaminase inhibitor precursors and small activating RNA (saRNA).

In some embodiments, the compound of Formula (II) or (I) is administered in combination with firsocostat and OCA. In some embodiments, the compound of Formula (II) or (I) is administered in combination with firsocostat and semaglutide. In some embodiments, the compound of Formula (II) or (I) is administered in combination with OCA and semaglutide. In some embodiments, the compound of Formula (II) or (I) is administered in combination with firsocostat, OCA and semaglutide.

In some embodiments, the third or more active agent is a dipeptidyl peptidase inhibitor (DPP-4 antagonist). DPP-4 antagonists are a class of oral hypoglycemics that block DPP-4 (DPP-IV). They can be used to treat diabetes mellitus type 2. Glucagon increases blood glucose levels, and DPP-4 inhibitors reduce glucagon and blood glucose levels. The mechanism of DPP-4 inhibitors is to increase incretin levels (GLP-1 and GIP), which inhibit glucagon release, which in turn increases insulin secretion, decreases gastric emptying, and decreases blood glucose levels. A non-limiting example list of dipeptidyl peptidase inhibitors includes: Sitagliptin, Vildagliptin, Saxagliptin, Linagliptin, Gemigliptin, Anagliptin, Teneligliptin, Alogliptin, Trelagliptin, Omarigliptin, Evogliptin, Dutogliptin.

In some embodiments, the third or more additional agent is an omega-3 fatty acid. When the third additional active agent is an omega-3 fatty acid, the omega-3 fatty acid is typically a long chain polyunsaturated omega-3 fatty acid (LC n-3 PUFA). Preferably, this includes at least one of (all-Z omega-3)-5,8,11,14,17-eicosapentaenoic acid (EPA) and (all-Z omega-3)-4,7,10,13,16,19-docosahexaenoic acid (DHA), or derivatives thereof. The n-3 PUFAs, including the EPA and DHA, may be in different forms, and are presented in at least one of free fatty acid form; esterified form, such as C1-C4 alkyl esters, and preferably ethyl ester; phospholipids; mono/di/tri-glycerides; and salts thereof. The omega-3 fatty acid may be provided in the form of a composition, such as a composition for oral administration. Such composition may comprise at least 40%, such as at least 50%, 60%, 70% or 80% of the active omega-3 fatty acid. In some embodiments, the third or more additional active agent is a composition comprising at least one of EPA and DHA, preferably on ethyl ester form, in a concentration of at least 70%.

In some embodiments, the third or more additional active agents are independently selected from the group of acetylsalicylic acid, alipogene tiparvovec, aramchol, atorvastatin, BI 1467335, BLX-1002, BMS-986036, BMS-986020, cenicriviroc, cobiprostone, colesevelam, emricasan, enalapril, foramulab, GFT-505, GR-MD-02, GS-0976, GS-9674, hydrochlorothiazide, icosapent ethyl ester (ethyl eicosapentaenoic acid, EPA ethyl ester), IMM-124E, IVA337, K-877, KD-025, linagliptin, liraglutide, mercaptamine, MGL-3196, ND-L02-s0201, obeticholic acid, olesox-ime, peg-ilodecakin, pioglitazone, PRM-151, PX-102, remogliflozin etabonate, selonsertib, simtuzumab, SHP-626, solithromycin, tipelukast, TRX-318, ursodeoxycholic acid, and VBY-376.

The third or more agent of the combined therapy be formulated as is suitable for the type of agent it is, and depends on several factors, including the mode of administration of the agent. The dose of the third or more additional active agent(s) depends on the type of agent selected, and it should be in accordance with the approved amounts for the specific agent.

The combination therapies of the present disclosure comprising compounds of Formula (II) or Formula (I) and at least one additional active agent chosen from a glucagon-like peptide 1 (GLP-1) receptor agonist, an acetyl-CoA carboxylase (ACC) inhibitor, and a farnesoid X receptor (FXR) agonist, may be administered to treat and/or reverse non-alcoholic steatohepatitis (NASH) or alcoholic steatohepatitis (ASH).

The present inventors have found that combination therapies comprising compounds of Formula (I), such as 2-(((5Z,8Z,11Z,14Z,17Z)-icosa-5,8,11,14,17-pentaen-1-yl)oxy)butanoic acid co-administered with at least one additional active agent chosen from a glucagon-like peptide 1 (GLP-1) receptor agonist, an acetyl-CoA carboxylase (ACC) inhibitor, and a farnesoid X receptor (FXR) agonist, have remarkably good pharmaceutical activity. The combination therapies disclosed exhibit unexpectedly improved biological activity compared to the monotherapies of active agents, for treating NASH associated hepatic fibrosis, inflammation, and steatosis.

### Examples

The present disclosure may be further described by the following non-limiting examples, in which standard techniques known to the skilled chemist and techniques analogous to those described in these examples may be used where appropriate. It is understood that the skilled artisan will envision additional embodiments consistent with the disclosure provided herein.

Unless otherwise stated, reactions were carried out at room temperature, typically in the range between 18-25° C. with solvents of HPLC grade under anhydrous conditions. Evaporations were carried out by rotary evaporation in vacuo. Column chromatography was performed by the flash procedure on silica gel 40-63 μm (Merck) or by an Armen Spot Flash using the pre-packed silica gel columns “MiniVarioFlash™”, “SuperVario Flash™”, “SuperVarioPrep™” or “EasyVarioPrep™” (Merck). Nuclear magnetic resonance (NMR) shift values were recorded on a Bruker Avance™ DPX 200 or 300 instrument with peak multiplicities described as follows: s, singlet; d, doublet; dd, double doublet; t, triplet; q, quartet; p, pentet; m, multiplett; br, broad. The mass spectra were recorded with a LC/MS spectrometer. Separation was performed using an Agilent 1100 series module on an Eclipse XDB-C18 2.1×150 mm column with gradient elution. As eluent were used a gradient of 5-95% acetonitrile in buffers containing 0.01% trifluoroacetic acid or 0.005% sodium formate. The mass spectra were recorded with a GI956A mass spectrometer (electrospray, 3000 V) switching positive and negative ionization mode. Reported yields are illustrative and do not necessarily represent the maximum yield attainable.

### Examples

### Example 1: Preparation of tert-butyl 2-((5Z,8Z,11Z,14Z,17Z)-icosa-5,8,11,14,17-pentaen-1-yloxy)butanoate

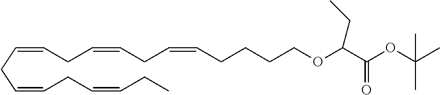 [SMILES: CC/C=C\C/C=C\C/C=C\C/C=C\C/C=C\CCCCOC(CC)C(=O)OC(C)(C)C]

Tetrabutylammonium chloride (0.55 g, 1.98 mmol) was added to a solution of (5Z,8Z,11Z,14Z,17Z)-icosa-5,8,11,14,17-pentaen-1-ol, (3.50 g, 12.1 mmol) in toluene (35 mL) at room temperature under nitrogen. An aqueous solution of sodium hydroxide (50% (w/w), 11.7 mL) was added under vigorous stirring at room temperature, followed by t-butyl 2-bromobutyrate (5.41 g, 24.3 mmol). The resulting mixture was heated to 50° C. and additional tbutyl 2-bromobutyrate was added after 1.5 hours (2.70 g, 12.1 mmol), 3.5 hours (2.70 g, 12.1 mmol) and 4.5 hours (2.70 g, 12.1 mmol) and stirred for 12 hours in total. After cooling to room temperature, ice water (25 mL) was added and the resulting two phases were separated. The organic phase was washed with a mixture of NaOH (5%) and brine, dried (MgSO₄), filtered and concentrated. The residue was purified by flash chromatography on silica gel using increasingly polar mixtures of heptane and ethyl acetate (100:0->95:5) as eluent. Concentration of the appropriate fractions afforded 1.87 g (36% yield) of the title compound as an oil. ¹H NMR (300 MHz, CDCl₃): δ 0.85-1.10 (m, 6H), 1.35-1.54 (m, 11H), 1.53-1.87 (m, 4H), 1.96-2.26 (m, 4H), 2.70-3.02 (m, 8H), 3.31 (dt, 1H), 3.51-3.67 (m, 2H), 5.10-5.58 (m, 10H).

### Example 2: Preparation of 2-((5Z,8Z,11Z,14Z,17Z)-icosa-5,8,11,14,17-pentaenyloxy)butanoic acid (Compound A)

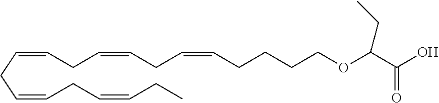 [SMILES: CC/C=C\C/C=C\C/C=C\C/C=C\C/C=C\CCCCOC(CC)C(=O)O]

tert-Butyl 2-((5Z,8Z,11Z,14Z,17Z)-icosa-5,8,11,14,17-pentaen-1-yloxy)butanoate (19.6 g, 45.5 mmol) was dissolved in dichloromethane (200 mL) and placed under nitrogen. Trifluoroacetic acid (50 mL) was added and the reaction mixture was stirred at room temperature for one hour. Water was added and the aqueous phase was extracted twice with dichloromethane. The combined organic extract was washed with brine, dried (Na₂SO₄), filtered and concentrated. The residue was subjected to flash chromatography on silica gel using increasingly polar mixtures of heptane, ethyl acetate and formic acid (90:10:1->80:20:1) as eluent. Concentration of the appropriate fractions afforded 12.1 g (71% yield) of the title compound as an oil. ¹H-NMR (300 MHz, CDCl₃): δ 0.90-1.00 (m, 6H), 1.50 (m, 2H), 1.70 (m, 2H), 1.80 (m, 2H), 2.10 (m, 4H), 2.80-2.90 (m, 8H), 3.50 (m, 1H), 3.60 (m, 1H), 3.75 (t, 1H), 5.30-5.50 (m, 10H); MS (electrospray): 373.2 [M-H]⁻.

Preparation of additional compounds of Formula I and Formula II of the present disclosure can be prepared according to the methods provided in PCT publication No. WO 2019/111048. For example, the following exemplary compounds can be prepared according to the published procedures listed as Examples 3-79 on pages 29-87 of WO 2019/111048, which are incorporated by reference herein.

TABLE 1
Exemplary compounds of Formulas (I) and (II)

| Structure | Name |
|---|---|
| 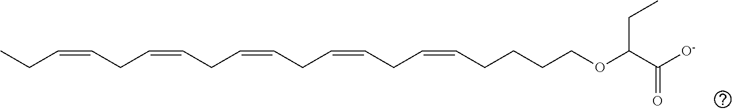 [SMILES: CC/C=C\C/C=C\C/C=C\C/C=C\C/C=C\CCCCOC(CC)C(=O)[O-]] | Calcium 2-(((5Z,8Z,11Z,14Z,17Z)-icosa-5,8,11,14,17- pentaen-1-yl)oxy)butanoate |
| 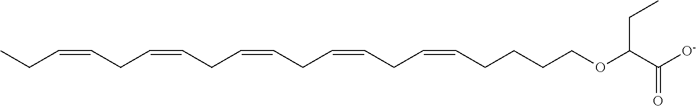 [SMILES: CC/C=C\C/C=C\C/C=C\C/C=C\C/C=C\CCCCOC(CC)C(=O)[O-]] | |
| 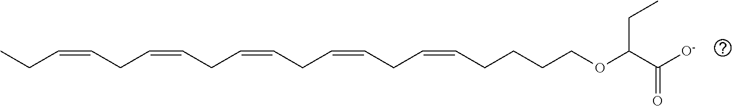 [SMILES: CC/C=C\C/C=C\C/C=C\C/C=C\C/C=C\CCCCOC(CC)C(=O)[O-]] | Sodium 2-(((5Z,8Z,11Z,14Z,17Z)-icosa-5,8,11,14,17- pentaen-1-yl)oxy)butanoate |
| 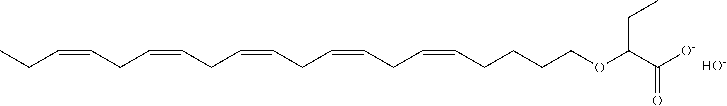 | 2-Hydroxy-N,N,N-trimethylethan-1-aminium 2-(((5Z,8Z,11Z,14Z,17Z)-icosa- 5,8,11,14,17-pentaen-1-yl)oxy)butanoate |
| 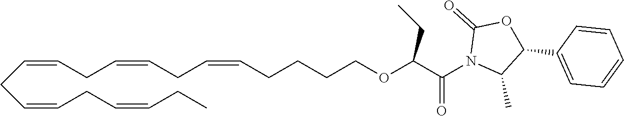 [SMILES: CC/C=C\C/C=C\C/C=C\C/C=C\C/C=C\CCCCO[C@@H](CC)C(=O)N1C(=O)O[C@H](c2ccccc2)[C@@H]1C] | (4S,5R)-3-((S)-2-((5Z,8Z,11Z,14Z,17Z)- icosa-5,8,11,14,17-pentaenyloxy)butanoyl)- 4-methyl-5-phenyloxazolidin-2-one and (4S,5R)-3-((R)-2-((5Z,8Z,11Z,14Z,17Z)- icosa-5,8,11,14,17-pentaenyloxy)butanoyl)- 4-methyl-5-phenyloxazolidin-2-one |
| 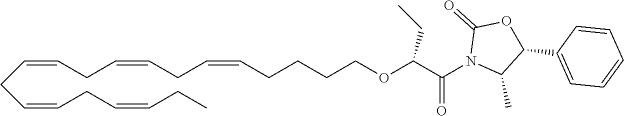 [SMILES: CC/C=C\C/C=C\C/C=C\C/C=C\C/C=C\CCCCO[C@H](CC)C(=O)N1C(=O)O[C@H](c2ccccc2)[C@@H]1C] | |
| 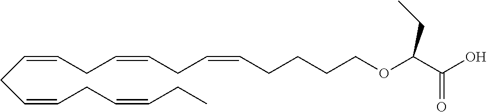 [SMILES: CC/C=C\C/C=C\C/C=C\C/C=C\C/C=C\CCCCO[C@@H](CC)C(=O)O] | (S)-2-((5Z,8Z,11Z,14Z,17Z)-icosa-5,8,11,14,17- pentaenyloxy)butanoic acid |
| 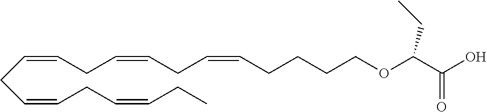 [SMILES: CC/C=C\C/C=C\C/C=C\C/C=C\C/C=C\CCCCO[C@H](CC)C(=O)O] | (R)-2-((5Z,8Z,11Z,14Z,17Z)-icosa-5,8,11,14,17- pentaenyloxy)butanoic acid |
| 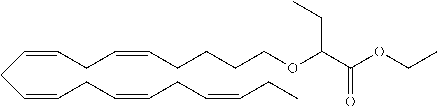 [SMILES: CC/C=C\C/C=C\C/C=C\C/C=C\C/C=C\CCCCOC(CC)C(=O)OCC] | Ethyl 2-(((5Z,8Z,11Z,14Z,17Z)-icosa- 5,8,11,14,17-pentaen-1-yl)oxy)butanoate |
| 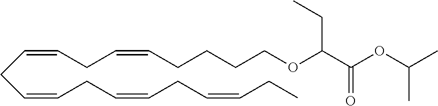 [SMILES: CC/C=C\C/C=C\C/C=C\C/C=C\C/C=C\CCCCOC(CC)C(=O)OC(C)C] | isopropyl 2-(((5Z,8Z,11Z,14Z,17Z)-icosa- 5,8,11,14,17-pentaein-1-yl)oxy)butanoate |
| 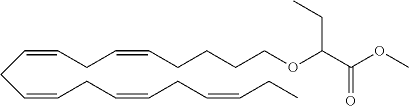 [SMILES: CC/C=C\C/C=C\C/C=C\C/C=C\C/C=C\CCCCOC(CC)C(=O)OC] | Methyl 2-(((5Z,8Z,11Z,14Z,17Z)-icosa- 5,8,11,14,17-pentaen-1-yl)oxy)butanoate |
| 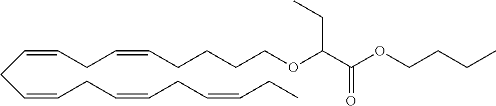 [SMILES: CC/C=C\C/C=C\C/C=C\C/C=C\C/C=C\CCCCOC(CC)C(=O)OCCCC] | Butyl 2-(((5Z,8Z,11Z,14Z,17Z)-icosa- 5,8,11,14,17-pentaen-1-yl)oxy)butanoate |
| 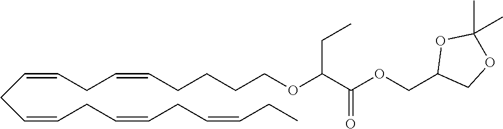 [SMILES: CC/C=C\C/C=C\C/C=C\C/C=C\C/C=C\CCCCOC(CC)C(=O)OCC1COC(C)(C)O1] | (2,2-Dimethyl-1,3-dioxolan-4-yl)methyl 2- (((5Z,8Z,11Z,14Z,17Z)-icosa-5,8,11,14,17- pentaen-1-yl)oxy)butanoate |
| 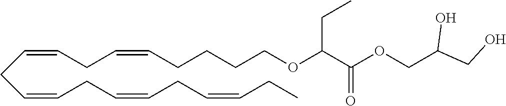 [SMILES: CC/C=C\C/C=C\C/C=C\C/C=C\C/C=C\CCCCOC(CC)C(=O)OCC(O)CO] | 2,3-Dihydroxypropyl 2-(((5Z,8Z,11Z,14Z,17Z)-icosa- 5,8,11,14,17-pentaen-1-yl)oxy)butanoate |
| 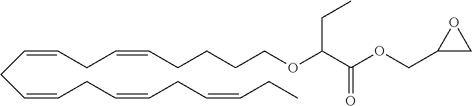 [SMILES: CC/C=C\C/C=C\C/C=C\C/C=C\C/C=C\CCCCOC(CC)C(=O)OCC1CO1] | Oxiran-2-ylmethyl 2-(((5Z,8Z,11Z,14Z,17Z)-icosa-5,8,11,14, 17-pentaen-1-yl)oxy)butanoate |
| 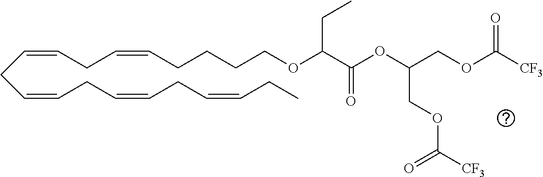 [SMILES: CC/C=C\C/C=C\C/C=C\C/C=C\C/C=C\CCCCOC(CC)C(=O)OC(COC(=O)C(F)(F)F)COC(=O)C(F)(F)F] | 2-((2-(((5Z,8Z,11Z,14Z,17Z)-icosa-5,8,11,14,17-pentaen-1- yl)oxy)butanoyl)oxy)propane-1,3-diyl bis(2,2,2-trifluoroacetate) |
| 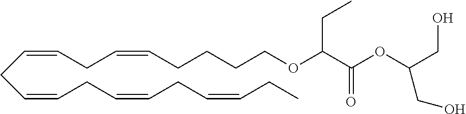 [SMILES: CC/C=C\C/C=C\C/C=C\C/C=C\C/C=C\CCCCOC(CC)C(=O)OC(CO)CO] | 1,3-Dihydroxypropan-2-yl 2- (((5Z,8Z,11Z,14Z,17Z)-icosa-5,8,11,14,17- pentaen-1-yl)oxy)butanoate |
| 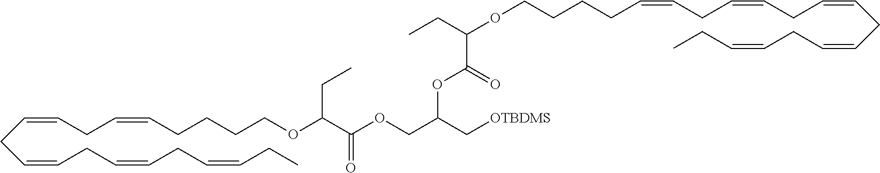 [SMILES: CC/C=C\C/C=C\C/C=C\C/C=C\C/C=C\CCCCOC(CC)C(=O)OCC(CO[Si](C)(C)C(C)(C)C)OC(=O)C(CC)OCCCC/C=C\C/C=C\C/C=C\C/C=C\C/C=C\CC] | 3-((Tert-butyldimethylsilyl)oxy)propane-1,2- diyl bis(2-(((5Z,8Z,11Z,14Z,17Z)-icosa- 5,8,11,14,17-pentaen-1-yl)oxy)butanoate) |
| 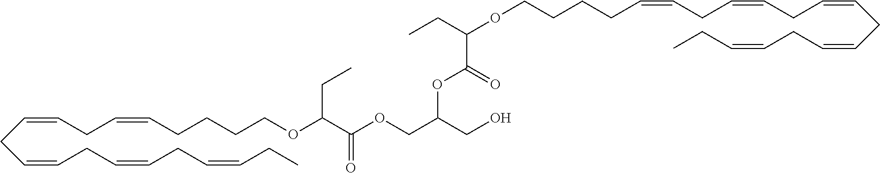 [SMILES: CC/C=C\C/C=C\C/C=C\C/C=C\C/C=C\CCCCOC(CC)C(=O)OCC(CO)OC(=O)C(CC)OCCCC/C=C\C/C=C\C/C=C\C/C=C\C/C=C\CC] | 3-Hydroxypropane-1,2-diyl bis(2- (((5Z,8Z,11Z,14Z,17Z)-icosa-5,8,11,14,17- pentaen-1-yl)oxy)butanoate) |
| 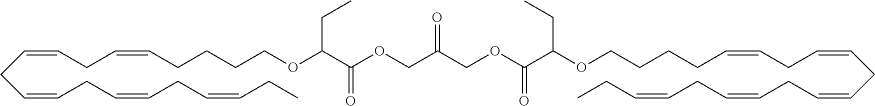 [SMILES: CC/C=C\C/C=C\C/C=C\C/C=C\C/C=C\CCCCOC(CC)C(=O)OCC(=O)COC(=O)C(CC)OCCCC/C=C\C/C=C\C/C=C\C/C=C\C/C=C\CC] | 2-Oxopropane-1,3-diyl bis(2- (((5Z,8Z,11Z,14Z,17Z)-icosa-5,8,11,14,17- pentaen-1-yl)oxy)butanoate) |
| 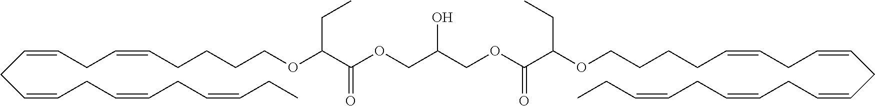 [SMILES: CC/C=C\C/C=C\C/C=C\C/C=C\C/C=C\CCCCOC(CC)C(=O)OCC(O)COC(=O)C(CC)OCCCC/C=C\C/C=C\C/C=C\C/C=C\C/C=C\CC] | 2-Hydroxypropane-1,3-diyl bis(2- (((5Z,8Z,11Z,14Z,17Z)-icosa-5,8,11,14,17-pentaen-1- yl)oxy)butanoate) |
| 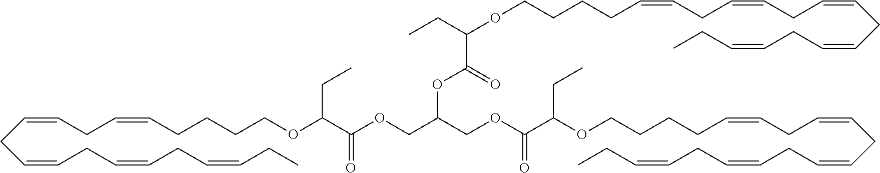 [SMILES: CC/C=C\C/C=C\C/C=C\C/C=C\C/C=C\CCCCOC(CC)C(=O)OCC(COC(=O)C(CC)OCCCC/C=C\C/C=C\C/C=C\C/C=C\C/C=C\CC)OC(=O)C(CC)OCCCC/C=C\C/C=C\C/C=C\C/C=C\C/C=C\CC] | Propane-1,2,3-triyl tris(2- (((5Z,8Z,11Z,14Z,17Z)-icosa-5,8,11,14,17-pentaen-1-yl)oxy) butanoate) |
| 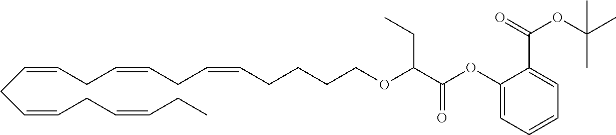 [SMILES: CC/C=C\C/C=C\C/C=C\C/C=C\C/C=C\CCCCOC(CC)C(=O)Oc1ccccc1C(=O)OC(C)(C)C] | Tert-butyl 2-((2-((5Z,8Z,11Z,14Z,17Z)- icosa-5,8,11,14,17-pentaen-1- yloxy)butanoyl)oxy)benzoate |
| 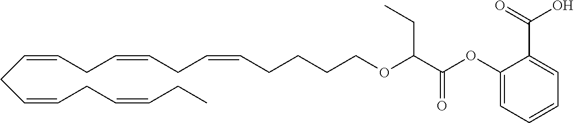 [SMILES: CC/C=C\C/C=C\C/C=C\C/C=C\C/C=C\CCCCOC(CC)C(=O)Oc1ccccc1C(=O)O] | 2-((2-((5Z,8Z,11Z,14Z,17Z)-icosa-5,8,11,14,17-pentaen-1- yloxy)butanoyl)oxy)benzoic acid |
| 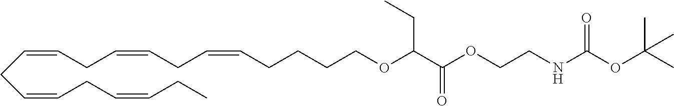 [SMILES: CC/C=C\C/C=C\C/C=C\C/C=C\C/C=C\CCCCOC(CC)C(=O)OCCNC(=O)OC(C)(C)C] | 2-((Tert-butoxycarbonyl)amino)ethyl 2- ((5Z,8Z,11Z,14Z,17Z)-icosa-5,8,11,14,17- pentaen-1-yloxy)butanoate |
| 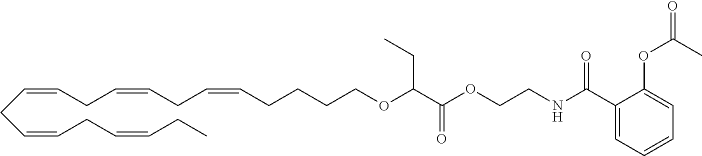 [SMILES: CC/C=C\C/C=C\C/C=C\C/C=C\C/C=C\CCCCOC(CC)C(=O)OCCNC(=O)c1ccccc1OC(C)=O] | 2-(2-Acetoxybenzamido)ethyl 2- ((5Z,8Z,11Z,14Z,17Z)-icosa-5,8,11,14,17- pentaen-1-yloxy)butanoate |
| 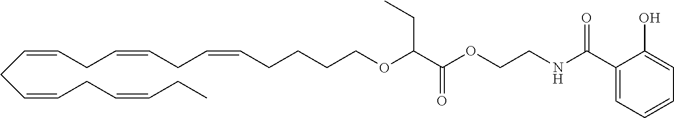 [SMILES: CC/C=C\C/C=C\C/C=C\C/C=C\C/C=C\CCCCOC(CC)C(=O)OCCNC(=O)c1ccccc1O] | 2-(2-Hydroxybenzamido)ethyl 2- ((5Z,8Z,11Z,14Z,17Z)-icosa-5,8,11,14,17- pentaen-1-yloxy)butanoate |
| 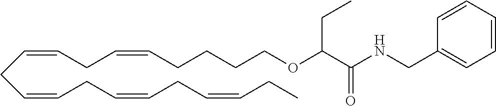 [SMILES: CC/C=C\C/C=C\C/C=C\C/C=C\C/C=C\CCCCOC(CC)C(=O)NCc1ccccc1] | N-benzyl-2-(((5Z,8Z,11Z,14Z,17Z)-icosa- 5,8,11,14,17-pentaen-1-yl)oxy)butanamide |
| 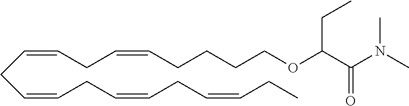 [SMILES: CC/C=C\C/C=C\C/C=C\C/C=C\C/C=C\CCCCOC(CC)C(=O)N(C)C] | 2-(((5Z,8Z,11Z,14Z,17Z)-icosa- 5,8,11,14,17-pentaen-1-yl)oxy)-N,N-dimethylbutanamide |
| 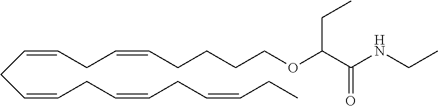 [SMILES: CC/C=C\C/C=C\C/C=C\C/C=C\C/C=C\CCCCOC(CC)C(=O)NCC] | N-ethyl-2-(((5Z,8Z,11Z,14Z,17Z)-icosa- 5,8,11,14,17-pentaen-1-yl)oxy)butanamide |
| 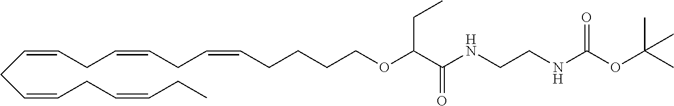 [SMILES: CC/C=C\C/C=C\C/C=C\C/C=C\C/C=C\CCCCOC(CC)C(=O)NCCNC(=O)OC(C)(C)C] | Tert-butyl (2-(2-(((5Z,8Z,11Z,14Z,17Z)- icosa-5,8,11,14,17-pentaen-1- yl)oxy)butanamido)ethyl)carbamate |
| 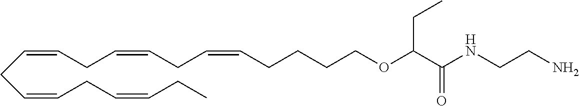 [SMILES: CC/C=C\C/C=C\C/C=C\C/C=C\C/C=C\CCCCOC(CC)C(=O)NCCN] | N-(2-aminoethyl)-2-(((5Z,8Z,11Z,14Z,17Z)- icosa-5,8,11,14,17-pentaen-1-yl)oxy)butanamide |
| 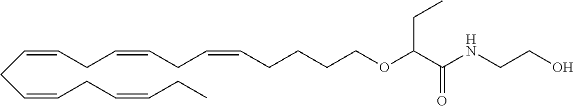 [SMILES: CC/C=C\C/C=C\C/C=C\C/C=C\C/C=C\CCCCOC(CC)C(=O)NCCO] | N-(2-hydroxyethyl)-2- (((5Z,8Z,11Z,14Z,17Z)-icosa-5,8,11,14,17- pentaen-1-yl)oxy)butanamide |
| 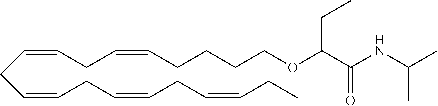 [SMILES: CC/C=C\C/C=C\C/C=C\C/C=C\C/C=C\CCCCOC(CC)C(=O)NC(C)C] | 2-(((5Z,8Z,11Z,14Z,17Z)-icosa- 5,8,11,14,17-pentaen-1-yl)oxy)-N-isopropylbutanamide |
| 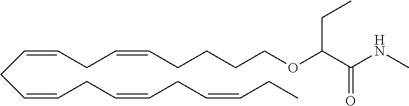 [SMILES: CC/C=C\C/C=C\C/C=C\C/C=C\C/C=C\CCCCOC(CC)C(=O)NC] | 2-(((5Z,8Z,11Z,14Z,17Z)-icosa- 5,8,11,14,17-pentaen-1-yl)oxy)-N-methylbutanamide |
| 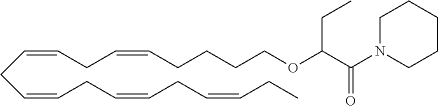 [SMILES: CC/C=C\C/C=C\C/C=C\C/C=C\C/C=C\CCCCOC(CC)C(=O)N1CCCCC1] | 2-(((5Z,8Z,11Z,14Z,17Z)-icosa-5,8,11,14,17-pentaen-1-yl) oxy)-1-(piperidin-1-yl)butan-1-one |
| 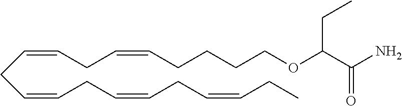 [SMILES: CC/C=C\C/C=C\C/C=C\C/C=C\C/C=C\CCCCOC(CC)C(N)=O] | 2-(((5Z,8Z,11Z,14Z,17Z)-icosa- 5,8,11,14,17-pentaen-1-yl)oxy)butanamide: |
| 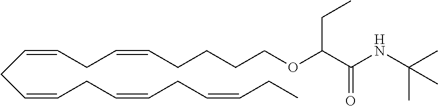 [SMILES: CC/C=C\C/C=C\C/C=C\C/C=C\C/C=C\CCCCOC(CC)C(=O)NC(C)(C)C] | N-(tert-butyl)-2-(((5Z,8Z,11Z,14Z,17Z)- icosa-5,8,11,14,17-pentaen-1- yl)oxy)butanamide |
| 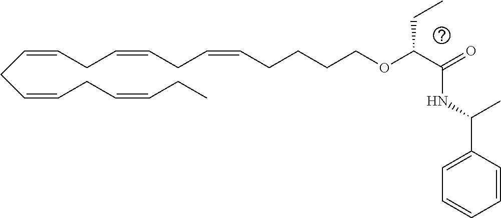 [SMILES: CC/C=C\C/C=C\C/C=C\C/C=C\C/C=C\CCCCO[C@H](CC)C(=O)N[C@H](C)c1ccccc1] | (R)-2-(((5Z,8Z,11Z,14Z,17Z)-icosa- 5,8,11,14,17-pentaen-1-yl)oxy)-N-((R)-1- phenylethyl)butanamide |
| 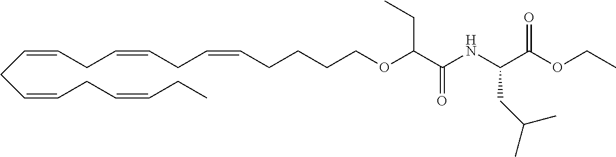 [SMILES: CC/C=C\C/C=C\C/C=C\C/C=C\C/C=C\CCCCOC(CC)C(=O)N[C@@H](CC(C)C)C(=O)OCC] | (2S)-ethyl 2-(2-((5Z,8Z,11Z,14Z,17Z)- icosa-5,8,11,14,17-pentaen-1- yloxy)butanamido)-4-methylpentanoate |
| 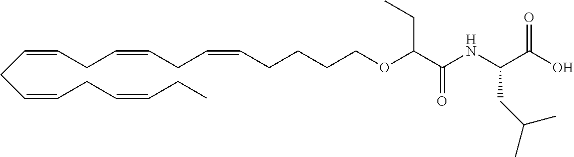 [SMILES: CC/C=C\C/C=C\C/C=C\C/C=C\C/C=C\CCCCOC(CC)C(=O)N[C@@H](CC(C)C)C(=O)O] | (2S)-2-(2-((5Z,8Z,11Z,14Z,17Z)-icosa- 5,8,11,14,17-pentaen-1-yloxy)butanamido)- 4-methylpentanoic acid |
| 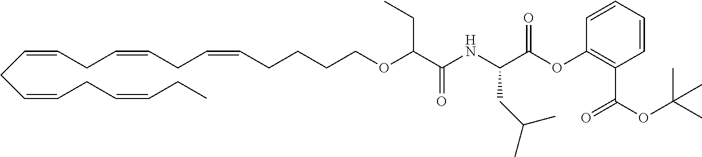 [SMILES: CC/C=C\C/C=C\C/C=C\C/C=C\C/C=C\CCCCOC(CC)C(=O)N[C@@H](CC(C)C)C(=O)Oc1ccccc1C(=O)OC(C)(C)C] | Tert-butyl 2-(((2S)-2-(2- ((5Z,8Z,11Z,14Z,17Z)-icosa-5,8,11,14,17- pentaen-1-yloxy)butanamido)-4- methylpentanoyl)oxy)benzoate |
| 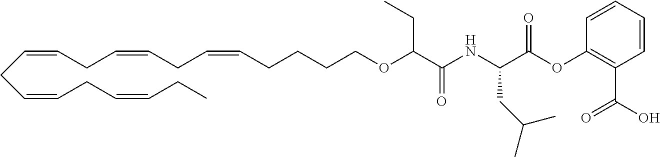 [SMILES: CC/C=C\C/C=C\C/C=C\C/C=C\C/C=C\CCCCOC(CC)C(=O)N[C@@H](CC(C)C)C(=O)Oc1ccccc1C(=O)O] | 2-(((2S)-2-(2-((5Z,8Z,11Z,14Z,17Z)-icosa- 5,8,11,14,17-pentaen-1-yloxy)butanamido)- 4-methylpentanoyl)oxy)benzoic acid |
| 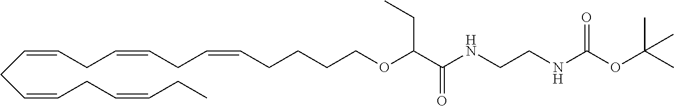 [SMILES: CC/C=C\C/C=C\C/C=C\C/C=C\C/C=C\CCCCOC(CC)C(=O)NCCNC(=O)OC(C)(C)C] | Tert-butyl (2-(2-((5Z,8Z,11Z,14Z,17Z)- icosa-5,8,11,14,17-pentaen-1- yloxy)butanamido)ethyl)carbamate |
| 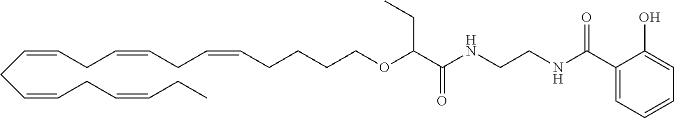 [SMILES: CC/C=C\C/C=C\C/C=C\C/C=C\C/C=C\CCCCOC(CC)C(=O)NCCNC(=O)c1ccccc1O] | 2-Hydroxy-N-(2-(2-((5Z,8Z,11Z,14Z,17Z)-icosa- 5,8,11,14,17-pentaen-1- yloxy)butanamido)ethyl)benzamide |
| 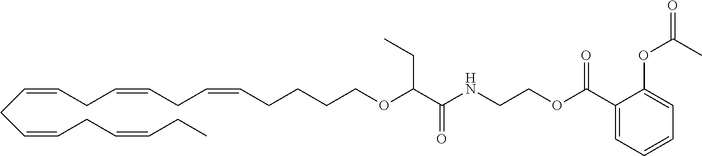 [SMILES: CC/C=C\C/C=C\C/C=C\C/C=C\C/C=C\CCCCOC(CC)C(=O)NCCOC(=O)c1ccccc1OC(C)=O] | 2-(2-((5Z,8Z,11Z,14Z,17Z)-icosa- 5,8,11,14,17-pentaen-1- yloxy)butanamido)ethyl 2-acetoxybenzoate |
| 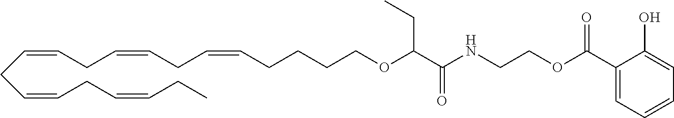 [SMILES: CC/C=C\C/C=C\C/C=C\C/C=C\C/C=C\CCCCOC(CC)C(=O)NCCOC(=O)c1ccccc1O] | 2-(2-((5Z,8Z,11Z,14Z,17Z)-icosa- 5,8,11,14,17-pentaen-1- yloxy)butanamido)ethyl 2-hydroxybenzoate |
| 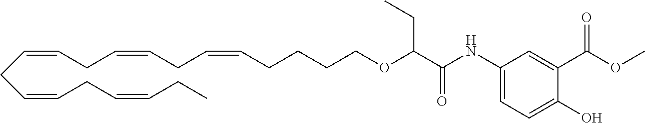 [SMILES: CC/C=C\C/C=C\C/C=C\C/C=C\C/C=C\CCCCOC(CC)C(=O)Nc1ccc(O)c(C(=O)OC)c1] | Methyl 2-hydroxy-5-(2- ((5Z,8Z,11Z,14Z,17Z)-icosa- 5,8,11,14,17- pentaen-1-yloxy)butanamido)benzoate |
| 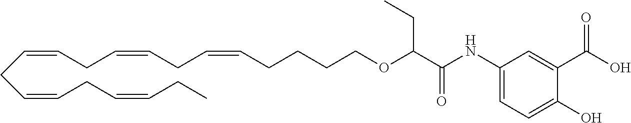 [SMILES: CC/C=C\C/C=C\C/C=C\C/C=C\C/C=C\CCCCOC(CC)C(=O)Nc1ccc(O)c(C(=O)O)c1] | 2-Hydroxy-5-(2-((5Z,8Z,11Z,14Z,17Z)-icosa- 5,8,11,14,17-pentaen-1- yloxy)butanamido)benzoic acid |
| 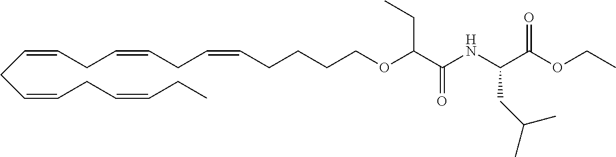 [SMILES: CC/C=C\C/C=C\C/C=C\C/C=C\C/C=C\CCCCOC(CC)C(=O)N[C@@H](CC(C)C)C(=O)OCC] | (2S)-ethyl 2-(2-((5Z,8Z,11Z,14Z,17Z)-icosa- 5,8,11,14,17-pentaen-1- yloxy)butanamido)-4-methylpentanoate |
| 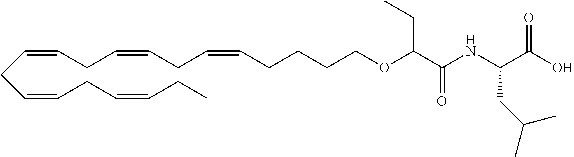 [SMILES: CC/C=C\C/C=C\C/C=C\C/C=C\C/C=C\CCCCOC(CC)C(=O)N[C@@H](CC(C)C)C(=O)O] | (2S)-2-(2-((5Z,8Z,11Z,14Z,17Z)-icosa- 5,8,11,14,17-pentaen-1-yloxy)butanamido)- 4-methylpentanoic acid |
| 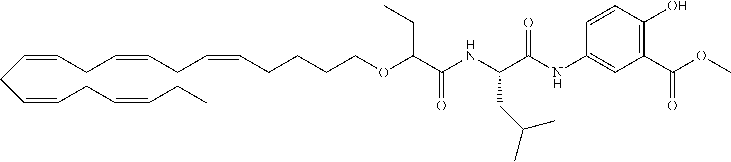 [SMILES: CC/C=C\C/C=C\C/C=C\C/C=C\C/C=C\CCCCOC(CC)C(=O)N[C@@H](CC(C)C)C(=O)Nc1ccc(O)c(C(=O)OC)c1] | Methyl 2-hydroxy-5-((2S)-2-(2- ((5Z,8Z,11Z,14Z,17Z)-icosa- 5,8,11,14,17- pentaen-1-yloxy)butanamido)-4- methylpentanamido)benzoate |
| 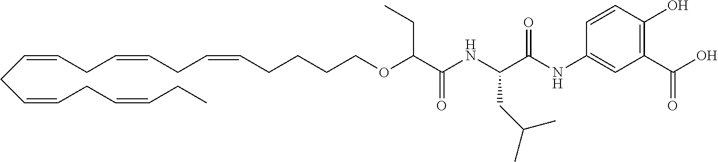 [SMILES: CC/C=C\C/C=C\C/C=C\C/C=C\C/C=C\CCCCOC(CC)C(=O)N[C@@H](CC(C)C)C(=O)Nc1ccc(O)c(C(=O)O)c1] | 2-Hydroxy-5-((2S)-2-(2- ((5Z,8Z,11Z,14Z,17Z)-icosa- 5,8,11,14,17- pentaen-1-yloxy)butanamido)-4- methylpentanamido)benzoic acid |
| 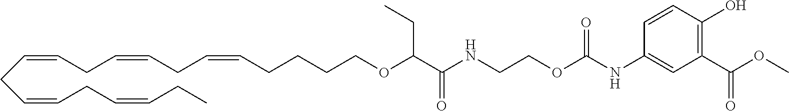 [SMILES: CC/C=C\C/C=C\C/C=C\C/C=C\C/C=C\CCCCOC(CC)C(=O)NCCOC(=O)Nc1ccc(O)c(C(=O)OC)c1] | Methyl 2-hydroxy-5-(((2-(2- ((5Z,8Z,11Z,14Z,17Z)-icosa- 5,8,11,14,17-pentaen-1- yloxy)butanamido)ethoxy)carbonyl)amino) benzoate |
| 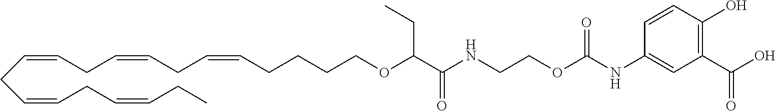 [SMILES: CC/C=C\C/C=C\C/C=C\C/C=C\C/C=C\CCCCOC(CC)C(=O)NCCOC(=O)Nc1ccc(O)c(C(=O)O)c1] | 2-Hydroxy-5-(((2-(2-((5Z,8Z,11Z,14Z,17Z)-icosa- 5,8,11,14,17-pentaen-1- yloxy)butanamido)ethoxy)carbonyl)amino) benzoic acid |
| 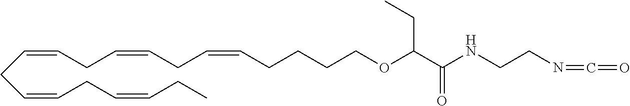 [SMILES: CC/C=C\C/C=C\C/C=C\C/C=C\C/C=C\CCCCOC(CC)C(=O)NCCN=C=O] | 2-((5Z,8Z,11Z,14Z,17Z)-icosa- 5,8,11,14,17-pentaen-1-yloxy)-N-(2- isocyanatoethyl)butanamide |
| 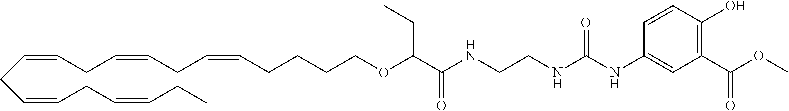 [SMILES: CC/C=C\C/C=C\C/C=C\C/C=C\C/C=C\CCCCOC(CC)C(=O)NCCNC(=O)Nc1ccc(O)c(C(=O)OC)c1] | Methyl 2-hydroxy-5-(3-(2-(2- ((5Z,8Z,11Z,14Z,17Z)-icosa-5,8,11,14,17-pentaen-1- yloxy)butanamido)ethyl)ureido)benzoate |
| 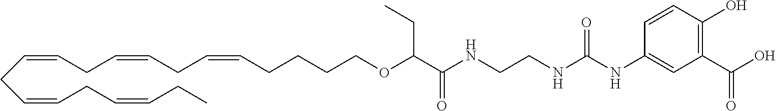 [SMILES: CC/C=C\C/C=C\C/C=C\C/C=C\C/C=C\CCCCOC(CC)C(=O)NCCNC(=O)Nc1ccc(O)c(C(=O)O)c1] | 2-Hydroxy-5-(3-(2-(2- ((5Z,8Z,11Z,14Z,17Z)-icosa- 5,8,11,14,17-pentaen-1- yloxy)butanamido)ethyl)ureido)benzoic acid |
| 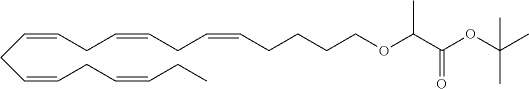 [SMILES: CC/C=C\C/C=C\C/C=C\C/C=C\C/C=C\CCCCOC(C)C(=O)OC(C)(C)C] | Tert-butyl 2-((5Z,8Z,11Z,14Z,17Z)-icosa- 5,8,11,14,17-pentaenyloxy)propanoate |
| 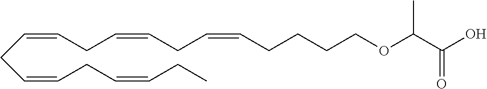 [SMILES: CC/C=C\C/C=C\C/C=C\C/C=C\C/C=C\CCCCOC(C)C(=O)O] | 2-((5Z,8Z,11Z,14Z,17Z)-icosa- 5,8,11,14,17-pentaenyloxy)propanoic acid |
| 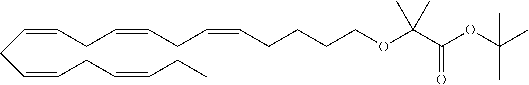 [SMILES: CC/C=C\C/C=C\C/C=C\C/C=C\C/C=C\CCCCOC(C)(C)C(=O)OC(C)(C)C] | Tert-butyl 2-((5Z,8Z,11Z,14Z,17Z)-icosa- 5,8,11,14,17-pentaenyloxy)-2- methylpropanoate |
| 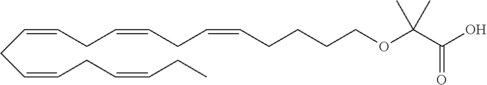 [SMILES: CC/C=C\C/C=C\C/C=C\C/C=C\C/C=C\CCCCOC(C)(C)C(=O)O] | 2-((5Z,8Z,11Z,14Z,17Z)-icosa- 5,8,11,14,17-pentaenyloxy)-2- methylpropanoic acid |
| 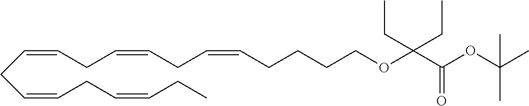 [SMILES: CC/C=C\C/C=C\C/C=C\C/C=C\C/C=C\CCCCOC(CC)(CC)C(=O)OC(C)(C)C] | Tert-butyl 2-ethyl-2-((5Z,8Z,11Z,14Z,17Z)-icosa- 5,8,11,14,17-pentaen-1- yloxy)butanoate |
| 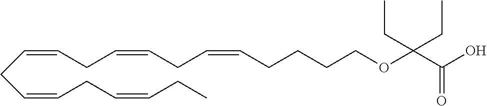 [SMILES: CC/C=C\C/C=C\C/C=C\C/C=C\C/C=C\CCCCOC(CC)(CC)C(=O)O] | 2-Ethyl-2-((5Z,8Z,11Z,14Z,17Z)-icosa- 5,8,11,14,17-pentaen-1-yloxy)butanoic acid |
| 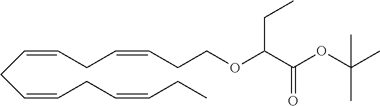 [SMILES: CC/C=C\C/C=C\C/C=C\C/C=C\CCOC(CC)C(=O)OC(C)(C)C] | Tert-butyl 2-(((3Z,6Z,9Z,12Z)-pentadeca- 3,6,9,12-tetraen-1-yl)oxy)butanoate |
| 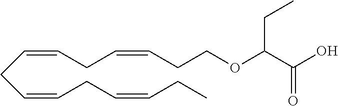 [SMILES: CC/C=C\C/C=C\C/C=C\C/C=C\CCOC(CC)C(=O)O] | 2-(((3Z,6Z,9Z,12Z)-pentadeca-3,6,9,12- tetraen-1-yl)oxy)butanoic acid |
| 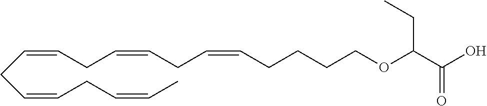 [SMILES: C/C=C\C/C=C\C/C=C\C/C=C\C/C=C\CCCCOC(CC)C(=O)O] | 2-(((5Z,8Z,11Z,14Z,17Z)-nonadeca- 5,8,11,14,17-pentaen-1-yl)oxy)butanoic acid |
| 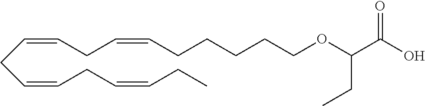 [SMILES: CC/C=C\C/C=C\C/C=C\C/C=C\CCCCCOC(CC)C(=O)O] | 2-(((6Z,9Z,12Z,15Z)-octadeca-6,9,12,15- tetraen-1-yl)oxy)butanoic acid |
| 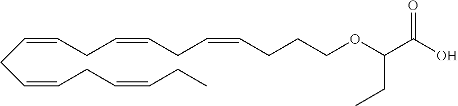 [SMILES: CC/C=C\C/C=C\C/C=C\C/C=C\C/C=C\CCCOC(CC)C(=O)O] | 2-(((4Z,7Z,10Z,13Z,16Z)-nonadeca- 4,7,10,13,16-pentaen-1-yl)oxy)butanoic acid |
| 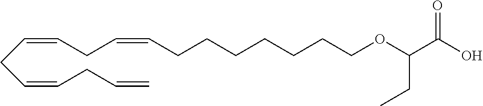 [SMILES: C=CC/C=C\C/C=C\C/C=C\CCCCCCCOC(CC)C(=O)O] | 2-(((8Z,11Z,14Z)-octadeca-8,11,14,17- tetraen-1-yl)oxy)butanoic acid |
| 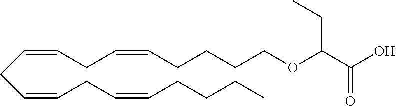 [SMILES: CCCC/C=C\C/C=C\C/C=C\C/C=C\CCCCOC(CC)C(=O)O] | 2-(((5Z,8Z,11Z,14Z)-nonadeca-5,8,11,14- tetraen-1-yl)oxy)butanoic acid |
| 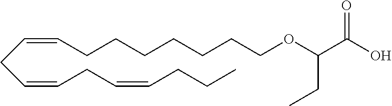 [SMILES: CCC/C=C\C/C=C\C/C=C\CCCCCCCOC(CC)C(=O)O] | 2-(((8Z,11Z,14Z)-octadeca-8,11,14-trien-1- yl)oxy)butanoic acid |
| 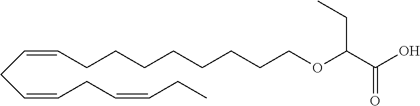 [SMILES: CC/C=C\C/C=C\C/C=C\CCCCCCCCOC(CC)C(=O)O] | 2-(((9Z,12Z,15Z)-octadeca-9,12,15-trien-1- yl)oxy)butanoic acid |
| 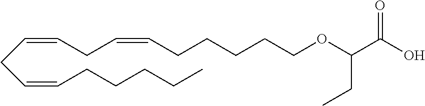 [SMILES: CCCCC/C=C\C/C=C\C/C=C\CCCCCOC(CC)C(=O)O] | 2-(((6Z,9Z,12Z)-octadeca-6,9,12-trien-1- yl)oxy)butanoic acid |
| 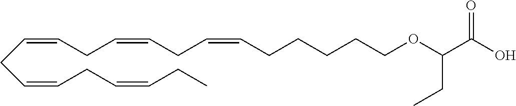 [SMILES: CC/C=C\C/C=C\C/C=C\C/C=C\C/C=C\CCCCCOC(CC)C(=O)O] | 2-(((6Z,9Z,12Z,15Z,18Z)-henicosa- 6,9,12,15,18-pentaen-1-yl)oxy)butanoic acid |
| 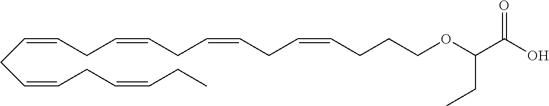 [SMILES: CC/C=C\C/C=C\C/C=C\C/C=C\C/C=C\C/C=C\CCCOC(CC)C(=O)O] | 2-(((4Z,7Z,10Z,13Z,16Z,19Z)-docosa- 4,7,10,13,16,19-hexaen-1-yl)oxy)butanoic acid |
| 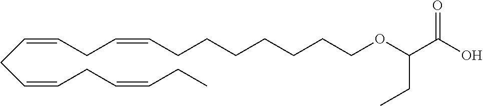 [SMILES: CC/C=C\C/C=C\C/C=C\C/C=C\CCCCCCCOC(CC)C(=O)O] | 2-(((8Z,11Z,14Z,17Z)-icosa-8,11,14,17- tetraen-1-yl)oxy)butanoic acid |
| 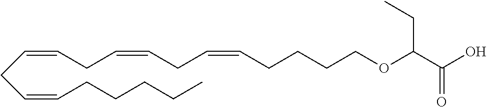 [SMILES: CCCCC/C=C\C/C=C\C/C=C\C/C=C\CCCCOC(CC)C(=O)O] | 2-(((5Z,8Z,11Z,14Z)-icosa-5,8,11,14- tetraen-1-yl)oxy)butanoic acid |
| 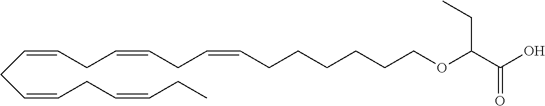 [SMILES: CC/C=C\C/C=C\C/C=C\C/C=C\C/C=C\CCCCCCOC(CC)C(=O)O] | 2-(((7Z,10Z,13Z,16Z,19Z)-docosa- 7,10,13,16,19-pentaen-1-yl)oxy)butanoic acid |
| 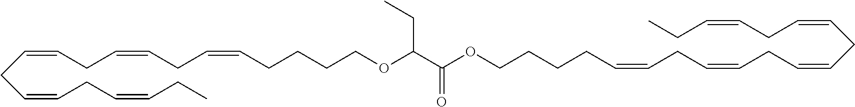 [SMILES: CC/C=C\C/C=C\C/C=C\C/C=C\C/C=C\CCCCOC(=O)C(CC)OCCCC/C=C\C/C=C\C/C=C\C/C=C\C/C=C\CC] | (5Z,8Z,11Z,14Z,17Z)-icosa-5,8,11,14,17- pentaen-1-yl 2-(((5Z,8Z,11Z,14Z,17Z)- icosa-5,8,11,14,17-pentaen-1-yl)oxy)butanoate |
| | (2R)-2-((2-((5Z,8Z,11Z,14Z,17Z)-icosa- |
| | 5,8,11,14,17-pentaen-1- |
| | yl)oxy)butanoyl)oxy)butanoic acid |
| 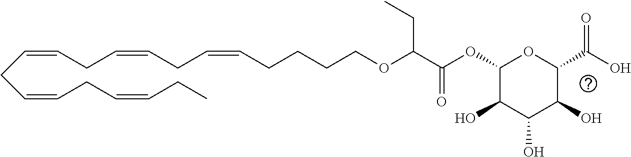 [SMILES: CC/C=C\C/C=C\C/C=C\C/C=C\C/C=C\CCCCOC(CC)C(=O)O[C@@H]1O[C@H](C(=O)O)[C@@H](O)[C@H](O)[C@H]1O] | (2S,3S,4S,5R,6S)-3,4,5-trihydroxy-6-((2- ((5Z,8Z,11Z,14Z,17Z)-icosa-5,8,11,14,17- pentaen-1-yl)oxy)butanoyl)oxy)tetrahydro- 2H-pyran-2-carboxylic acid |
| | 2-((5Z,8Z,11Z,14Z,17Z)-icosa- |
| | 5,8,11,14,17-pentaen-1-yl)oxy)butan-1-ol |
| | 2-((5Z,8Z,11Z,14Z,17Z)-icosa- |
| | 5,8,11,14,17-pentaen-1-yl)oxy)butyl acetate |

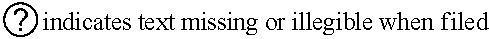

### Biological Examples

Abbreviations used:
α-SMA: alpha-smooth muscle actin
ALT: Alanine transaminase
AST: Aspartate transaminase
BW: Body weight
CDAA/HFD: choline-deficient high-fat diet
Col1a1: collagen 1a1
FI: Food intake
Gal-3: Galectin-3
HE or H&E: Hematoxylin and eosin
HP: Hydroxyproline
IHC: Immunohistochemistry
PSR: Picrosirius red or picro Sirius red
PO: per oral gavage
QD: Once a day
QW: Once a week
SC: Subcutaneous
SEM: Standard error of the mean
TC: Total cholesterol
TG: Triglycerides
   Evaluation of Compound a Alone and in Combination with Semaglutide, Firsocostat, and Obeticholic Acid in a Diet Induced NASH Mouse Model (CDAA/High-Fat Diet)

### Animal Model

Mouse models that are deficient in methionine and/choline (MCD and CDAA respectively) are well established for studying the development and treatment of NASH in pre-clinical drug development. As precursors for the synthesis of phosphatidylcholine, a dietary deficiency of methionine/choline leads to an inability to synthesize hepatic lipoproteins for the export of triglyceride that results in severe hepatic steatosis, inflammation, and fibrosis.

Although the widely used methionine-choline deficient (MCD) diet consistently reproduces severe NASH-like hepatic inflammation and fibrosis in mice, it is also associated with severe weight-loss (loss of both skeletal muscle and fat mass). This is associated with an increased risk of death which presents major problems for long-term fibrogenesis experiments. By adding a sub-optimal dose of methionine (0.1%), the CDAA dietary model overcomes these problems and has been demonstrated to mimic human NASH in both mice and rats by sequentially producing steatohepatitis, liver fibrosis and liver cancer with less severe loss of body weight. In the current study, male mice (strain C57BI/6JRj) were fed choline deficient high-fat diets (45% of total calories from fat; “CDAA/high-fat” or “CDAA-HFD”). The NASH inducing diet was instigated 6 weeks prior to the commencement of administration of active agents in order to evaluate treatment and reversal of NASH parameters. The NASH inducing CDAA-HFD was continued for the 8 weeks of administration of the indicated active agents and combinations thereof.

The aim of this study was to evaluate the effects of 8 weeks of administration of Compound A, OCA, semaglutide, and firsocostat alone and in combination on metabolic parameters, hepatic pathology, and on NAFLD Activity Score including Fibrosis Stage in male CDAA-HFD mice.

Prior to administration of the activate agents, animals were randomized into treatment groups based on body weight. A baseline group (n=12) was terminated at study start after 6 weeks on the CDAA-HFD diet. CDAA-HFD fed mice (n=10-12 per group) received daily per oral (PO) treatment with vehicle, Compound A (112 mg/kg), OCA (30 mg/kg), semaglutide (30 nmol/kg SC), firsocostat (5 mg/kg), Compound A+Semaglutide (112 mg/kg+30 nmol/kg), Compound A+OCA (112 mg/kg+30 mg/kg), and Compound A+firsocostat (112 mg/kg+5 mg/kg) for 8 weeks. Terminal liver biopsy were analyzed for histopathological scores. Terminal quantitative endpoints included plasma/liver biochemistry and liver histomorphometry.

TABLE 2
Experimental Groups

| | | | | Dosing & |
|---|---|---|---|---|
| | | | Dose | Route of |
| | Group | Compound | (mg/kg) | Administration |
| 1 | Baseline | NA | NA | NA |
| 2 | Vehicle 1 + | Corn oil + | NA | PO + SC |
| | Vehicle 2 | 50 mM PBS + | | |
| | | 5% mannitol | | |
| 3 | Compound A + | Compound A + | 112 mg/kg | PO + SC |
| | Vehicle 2 | 50 nM PBS + | | |
| | | 5% mannitol | | |
| 4 | OCA + | OCA + | 30 mg/kg | PO + SC |
| | Vehicle 2 | 50 nM PBS + | | |
| | | 5% mannitol | | |
| 5 | Semaglutide + | Semaglutide + | 30 nmol/kg* | PO + SC |
| | Vehicle 1 | Corn oil* | | |
| 6 | Firsocostat + | Firsocostat + | 5 mg/kg | PO + SC |
| | Vehicle 2 | 50 nM PBS + | | |
| | | 5% mannitol | | |
| 7 | Compound A + | Compound A + | 112 mg/kg + | PO + SC |
| | Semaglutide | Semaglutide | 30 nmol/kg* | |
| 8 | Compound A + | Compound A + | 112 mg/kg + | PO + PO |
| | OCA | OCA | 30 mg/kg* | |
| 9 | Compound A + | Compound A + | 112 mg/kg + | PO + PO |
| | Firsocostat | Firsocostat | 5 mg/kg | |

Model = CDAAHFD; n per group = 12; Dosing Frequency = QD; Dosing days (inclusive) = 0 to 55/56, Dosing volume = 5 ml/kg;
*Titration: 0.6 nmol/kg day 1; 1.2 nmol/kg day 2; 2.4 nmol/kg day 3: 4.8 nmol/kg day 4; 12 nmol/kg day 5, 30 nmol/kg day 6

### NAFLD Activity Score (NAS) and Fibrosis Stage Score

Liver samples stained with Hematoxylin and Eosin (H&E) or Picro Sirius Red (PSR) were scored for NAS components (steatosis, lobular inflammation, and ballooning degeneration) and fibrosis stage respectively using the clinical criteria outlined by Kleiner et al., Hepatology, 2005; 41. Total NAS represents the sum of scores for steatosis, inflammation, and ballooning, and ranges from 0-8.

TABLE 3
NAS and Fibrosis Stage Scoring

| Feature | Degree | Score |
|---|---|---|
| Steatosis | <5% | 0 |
| (percentage of | 5-33% | 1 |
| hepatocytes with | >33-66% | 2 |
| lipid droplets) | >66% | 3 |
| Lobular | No foci | 0 |
| inflammation | <2 foci/200x | 1 |
| | 2-4 foci/200x | 2 |
| | >4 foci/200x | 3 |
| Ballooning | None | 0 |
| degeneration | Few | 1 |
| | Many cells/prominent ballooning | 2 |
| Fibrosis | None | 0 |
| | Perisinusoidal or periportal | 1 |
| | Perisinusoidal & portal/periportal | 2 |
| | Bridging fibrosis | 3 |
| | Cirrhosis | 4 |

NAS and fibrosis stage was determined by deep learning applications developed by Gubra (Denmark) using the VIS software (Visiopharm®, Denmark) for a more accurate and objective method for staging disease in DIO-NASH mouse models.

### Steatosis, Inflammation, and Ballooning Degeneration:

Scanned H&E stained slides were analyzed in several steps:
1. Tissue detection at low magnification, followed by deep learning-based detection of portal triads and central veins at 10× magnification.
2. Another deep learning application detected nuclei of hepatocytes with lipid droplets, hepatocytes without lipids, ballooned hepatocytes, and inflammatory cells at 20× magnification. Inflammatory foci were defined as a cluster of >3 inflammatory cells.
3. The inflammation score of the liver tissue sample was the average score for all 20× fields.

Steatosis score was calculated as percentage of hepatocytes with lipid droplets.

### Fibrosis Stage:

Scanned PSR stained slides were analyzed in several steps:
1. Tissue detection at low magnification, followed by deep learning-based detection of portal triads and central veins at 10× magnification. Periportal zone was defined as 100 μm around the portal triads.
2. Fibrosis fibers were detected using the linear Bayesian image analysis method in the periportal and sinusoidal zones.
3. Bridging was detected using threshold image analysis based on a polynomial local linear filter feature.
4. Different measures of size, shape and connectivity form both fibrosis and bridging fibrosis in the two zoned were used in a XGBoost classifier to predict fibrosis stage.

### Quantitative Assessment of Immunoreactivity and Histochemical Staining

Immunohistochemistry (IHC)-positive staining was quantified by image analysis using the VIS software (Visiopharm®, Denmark) using two steps:
1. Detection of tissue at low magnification (1× objective), excluding the liver capsule; and
2. Detection of IHC-positive staining.

The quantitation of IHC-positive staining is calculated as an area fraction as follows:

$$Area {fraction}_{IHC - positive} = \frac{{Area}_{IHC - positive}}{{Area}_{Fat} + {Area}_{T i s s u e} + {Area}_{IHC - positive}}$$

IHC factional area quantification was assessed for galectin-3, collagen 1A1, and α-smooth muscle actin.

Hepatic factional area assessment with picrosirius red stain was determined using the same methods.

### Biological Example 1. Relative Body Weight and Relative Liver Weight

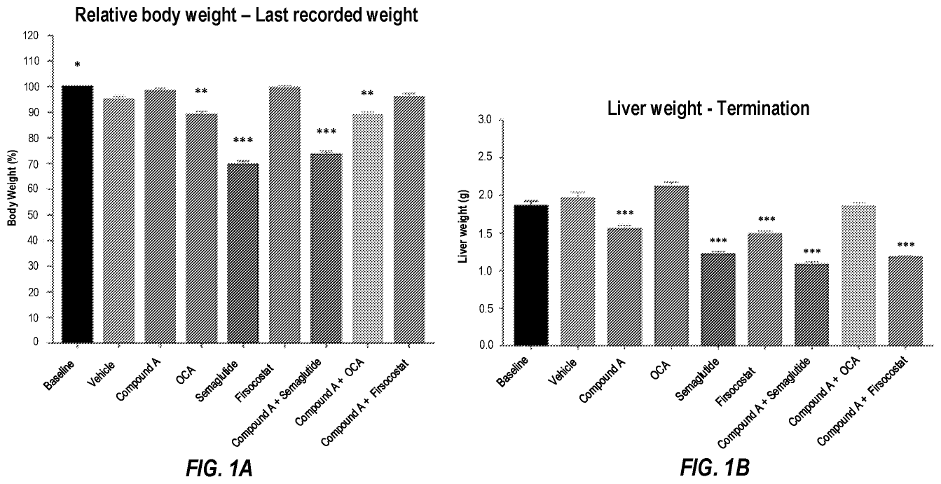

As shown in FIG. 1A, experimental groups that received OCA, Semaglutide, and Compound A+OCA, and Compound A+semaglutide had decreased last recorded relative body weight compared to vehicle. As shown in FIG. 1B, groups administered Compound A, semaglutide, Compound A+firsocostat, and Compound A+semaglutide had decreased liver weight when compared to the untreated vehicle group.

In FIGS. 1A and 1B, values are expressed as mean of n=9-12+SEM in a Dunnett's test one-factor linear model where *=P<0.05, **=P<0.01, and ***=P<0.001, when compared to the untreated vehicle group.

### Biological Example 2. Plasma AST and ALT Levels

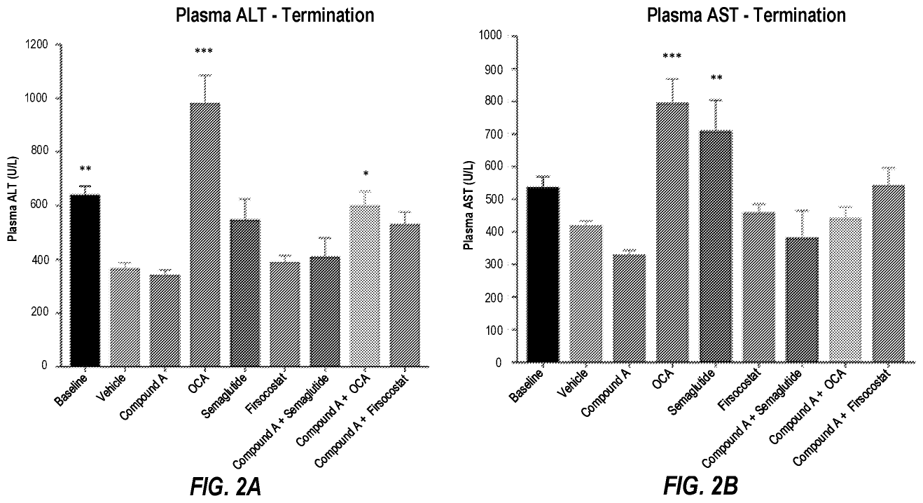

Plasma alanine aminotransferase (ALT) (FIG. 2A) and aspartate transaminase (AST) (FIG. 2B) were measured after 8 weeks of administration of Compound A, OCA, semaglutide, firsocostat, and combination therapies.

In FIGS. 2A and B, values are expressed as mean of n=9-12+SEM in a Dunnett's test one-factor linear model where *=P<0.05, **=P<0.01, and ***=P<0.001, when compared to the untreated vehicle group.

### Biological Example 3. Plasma Triglycerides and Total Cholesterol

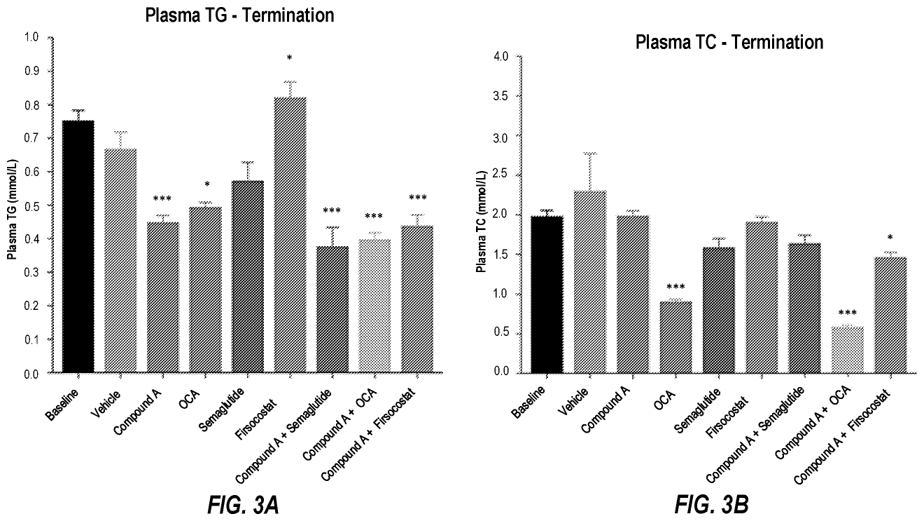

As shown in FIG. 3A, groups administered Compound A and OCA, and all of the combination therapy groups, had significantly decreased levels of plasma triglycerides. In the case of plasma levels of total cholesterol, as shown in FIG. 3B, monotherapy with OCA significantly decreased plasma cholesterol, as did the combination therapies Compound A+firsocostat and Compound A+OCA.

In FIGS. 3A & 3B, values are expressed as mean of n=7-12+SEM in a Dunnett's test one-factor linear model where *=P<0.05, **=P<0.01, and ***=P<0.001, when compared to the untreated vehicle group. Combination therapies were compared to Compound A alone in a Dunnett's test one-factor linear model where &=P<0.05, &&=P<0.01, and &&&=P<0.001. Combination therapies were also compared to their respective additional active agent (e.g., Compound A+semaglutide vs. semaglutide) in a Dunnett's test one-factor linear model where \hat{ }=P<0.05, \hat{ }\hat{ }=P<0.01, and \hat{ }\hat{ }=P<0.001.

### Biological Example 4. Hepatic Steatosis

### Liver Total Cholesterol and Triglycerides

As shown in FIG. 4A, groups administered Compound A, OCA, Compound A+firsocostat, Compound A+OCA, and Compound A+semaglutide showed a significant decrease in relative levels of liver total cholesterol (normalized to liver weight) when compared to the untreated vehicle group. The combination therapy groups all showed a significantly larger decrease than the groups receiving treatment with a single compound. In particular, while semaglutide alone did not significantly affect relative liver total cholesterol levels compared to the vehicle control, the combination of CompoundA+semaglutide had a significantly greater effect on relative liver total cholesterol levels compared to Compound A alone.

As shown in FIG. 4B, groups administered Compound A, OCA, Compound A+firsocostat, Compound A+OCA, and Compound A+semaglutide showed a significant decrease in relative levels liver triglycerides (normalized to liver weight) when compared to the untreated group. The groups receiving Compound A+semaglutide and Compound A+firsocostat both show a significantly larger decrease in liver triglyceride levels compared to the groups receiving only Compound A or only the respective additional active agent (i.e., semaglutide or firsocostat, respectively).

In FIGS. 4A & 4B, values are expressed as mean of n=7-12+SEM in a Dunnett's test one-factor linear model where *=P<0.05, **=P<0.01, and ***=P<0.001, when compared to the untreated vehicle group. Combination therapies were compared to Compound A alone in a Dunnett's test one-factor linear model where &=P<0.05, &&=P<0.01, and &&&=P<0.001. In FIG. 4B, combination therapies were also compared to their respective additional active agent (e.g., Compound A+semaglutide vs. semaglutide) in a Dunnett's test one-factor linear model where \hat{ }=P<0.05, \hat{ }\hat{ }=P<0.01, and \hat{ }\hat{ }=P<0.001.

### Liver Lipids

Hepatic steatosis area (relative liver lipids) was quantified on H&E stained slides by image analysis using the VIS software (Visiopharm®, Denmark). VIS protocols are designed to analyze the virtual slides in two steps:
1. Tissue detection at low magnification (1× objective); and
2. Detection of steatosis and tissue at high magnification (20× objective).

The quantitative estimates of steatosis were calculated as an area fraction as follows:

$$Area {fraction}_{Steatosis} = \frac{{Area}_{Steatosis}}{{Area}_{T i s s u e} + {Area}_{Steatosis}}$$

As shown in FIG. 5A, groups administered Compound A, OCA, and the respective combination therapies showed a significant relative decrease in hepatic steatosis area when compared to the untreated vehicle group. All of the combination therapy groups showed a significantly larger decrease compared to groups treated with single compounds. In particular, Compound A+firsocostat significantly reduced hepatic steatosis area by a much greater extent than either compound alone. Additionally, while semaglutide did not have a significant effect on the hepatic steatosis area compared to vehicle, the combination of Compound A+semaglutide resulted in significantly lower hepatic steatosis area than Compound A alone.

As shown in FIG. 5A, the vehicle control group had a significantly lower hepatic steatosis area as compared to baseline, and the decreases obtained with the combination therapies, as well as Compound A, OCA, and firsocostat alone, were all significantly greater than the untreated vehicle group.

As shown in FIG. 5B, groups administered Compound A, firsocostat, and all of the combination therapy groups had significantly decreased percentages of hepatocytes with lipid droplets when compared to the untreated vehicle group. The percentage of hepatocytes with lipid droplets was determined using deep learning-based image analysis.

The groups receiving Compound A+semaglutide and Compound A+firsocostat had significantly larger decreases in percentages of hepatocytes with lipid droplets than the group receiving the respective individual agents alone. In particular, while semaglutide alone did not significantly affect the percentage of hepatocytes with lipid droplets compared to the vehicle control, the combination of CompoundA+semaglutide had a significantly greater effect than did Compound

A alone. Additionally, the combination of Compound A+firsocostat had a much greater effect on the percentage of hepatocytes with lipid droplets than did either agent alone.

In FIGS. 5A & 5B, values are expressed as mean of n=7-12+SEM in a Dunnett's test one-factor linear model where *=P<0.05, **=P<0.01, and ***=P<0.001, when compared to the untreated vehicle group. Combination therapies were compared to Compound A alone in a Dunnett's test one-factor linear model where &=P<0.05, &&=P<0.01, and &&&=P<0.001. Combination therapies were also compared to their respective additional active agent (e.g., Compound A+semaglutide vs. semaglutide) in a Dunnett's test one-factor linear model where \hat{ }=P<0.05, \hat{ }\hat{ }=P<0.01, and \hat{ }\hat{ }=P<0.001.

### Histopathology Analysis-Steatosis Scores

As shown in FIG. 6, groups treated with Compound A, firsocostat, Compound A+firsocostat, and Compound A+semaglutide showed decreased steatosis scores when compared to the untreated vehicle group. For each group shown in FIG. 6, the number of animals with each score (1, 2, or 3) is indicated by the height of the stacked bars in a Mann-Whitney U test with Bonferroni correction where **=P<0.01 and ***=P<0.001, when compared to the untreated vehicle group.

When the data was analyzed for comparing the combination therapy treatment groups against the treatment group receiving only Compound A, in a Mann-Whitney U test with Bonferroni correction (&&&=P<0.001), the group administered Compound A+firsocostat showed a significant decrease in steatosis scores compared to the group receiving Compound A alone.

### Biological Example 6. Hepatic Inflammation

The number of inflammatory cells and inflammatory foci were determined by deep learning-based image analysis. As shown in FIG. 7A, the group administered Compound A and all three combination therapy groups had a significant decrease in the number of inflammatory cells (per mm²) when compared to the untreated vehicle group, as well as when compared to the baseline levels. The group treated with Compound A+semaglutide had a significantly larger decrease in inflammatory cells compared to the group administered Compound A alone, despite semaglutide alone not having a significant effect compared to vehicle and baseline.

FIG. 7B shows that groups administered Compound A and the combination therapies had a significant decrease in inflammatory foci (per mm²) when compared to the untreated vehicle group and compared to the baseline levels. The group treated with Compound A+semaglutide had a significantly larger decrease in inflammatory foci compared to the group administered Compound A alone, despite semaglutide alone not having a significant effect compared to vehicle and baseline.

The significant changes in the numbers of inflammatory cells and inflammatory foci for Compound A and each of the combination therapies compared to baseline indicate a regression effect, or reversal of inflammation that developed prior to the administration of Compound A alone or as a combination therapy.

In FIGS. 7A & 7B, values are expressed as mean of n=7-12+SEM in a Dunnett's test one-factor linear model where *=P<0.05, **=P<0.01, and ***=P<0.001, when compared to the untreated vehicle group, and where #=P<0.05, ##=P<0.01, and ###=P<0.001, when compared to the baseline level. Combination therapies were compared to Compound A alone in a Dunnett's test one-factor linear model where &=P<0.05, &&=P<0.01, and &&&=P<0.001.

### Histopathology Analysis-Lobular Inflammation and NAFLD Activity Scores

FIG. 8A, shows the clinical lobular inflammation scores for all of the treatment groups. FIG. 8B shows that groups administered Compound A, firsocostat, Compound A+firsocostat, and Compound A+semaglutide had significantly decreased NAFLD activity scores (NAS) when compared to the untreated vehicle group. When the combination therapy groups were compared to the group receiving only Compound A, the groups receiving Compound A+semaglutide and Compound A+firsocostat showed a significantly larger decrease in NAFLD scores than the Compound A group.

In FIGS. 8A and 8B, for each group, the number of animals with each score is indicated by the height of the stacked bars in a Mann-Whitney U test with Bonferroni correction. In FIG. 8A, there was no differences at a significance level of 0.05, and in FIG. 8B, **=P<0.01, when compared to the untreated vehicle group.

### Biological Example 7. Hepatic Fibrosis

### Fibrosis Markers

As shown in FIG. 9A, all of the treatment groups except the group administered OCA showed a significant decrease in relative liver hydroxyproline content (normalized to liver weight) when compared to the untreated vehicle group. The combination therapies Compound A+semaglutide and Compound A+firsocostat had significantly greater reductions in relative liver hydroxyproline content than did any of the respective agents alone.

Hepatic area expressing galectin-3, which is a marker of hepatic inflammation and fibrosis, was determined by histological quantitative assessment. As shown in FIG. 9B, groups administered

Compound A and all of the combination therapy groups had significantly decreased galectin-3 hepatic fractional area when compared to the untreated vehicle group. The Compound A+semaglutide combination therapy group had a significantly greater decrease in hepatic fractional area expressing galectin-3 compared with Compound A alone, despite semaglutide alone not having a significant effect compared to vehicle. Additionally, this combination showed a regression effect such that it significantly reduced relative galectin-3 area below that of baseline, despite the semaglutide group having significantly increased galectin-3 expressing area compared to baseline. The combination of Compound A and firsocostat also significantly reduced relative galectin-3 expressing area below baseline.

In FIGS. 9A & 9B, values are expressed as mean of n=7-12+SEM in a Dunnett's test one-factor linear model where *=P<0.05, **=P<0.01, and ***=P<0.001, when compared to the untreated vehicle group. Combination therapies were compared to Compound A alone in a Dunnett's test one-factor linear model where &=P<0.05, &&=P<0.01, and &&&=P<0.001. In FIG. 9A, combination therapies were compared to the respective additional active agent in a Dunnett's test one-factor linear model where \hat{ }\hat{ }=P<0.01 and \hat{ }\hat{ }\hat{ }=P<0.001. In FIG. 9B, each group was compared to the baseline level in a Dunnett's test one-factor linear model where #=P<0.05, ##=P<0.01, and ###=P<0.001.

### Hepatic Fibrotic Area—PSR

Hepatic fibrotic area was determined by staining with picrosirus red (PSR), which binds collagen, and histological quantitative assessment.

As shown in FIG. 10A, groups administered Compound A and combination therapies had significantly decreased hepatic fibrotic area as determined by PSR staining when compared to the untreated vehicle group. The reduction in hepatic fibrotic area obtained by the combination therapy Compound A+semaglutide was significantly greater than that obtained by Compound A alone. Semaglutide alone did not significantly affect hepatic fibrotic area.

Sinusoidal and periportal fibrotic area was determined using PSR and histological quantitative assessment and deep learning image analysis. As shown in FIG. 10B, the Compound A+semaglutide significantly decreased sinusoidal fibrotic area when compared to the untreated vehicle group. Neither compound alone had a significant effect. FIG. 10C shows the periportal fibrotic area for the various experimental groups.

In FIGS. 10A, 10B, and 10C, values are expressed as mean of n=9-12+SEM in a Dunnett's test one-factor linear model where *=P<0.05, **=P<0.01, and ***=P<0.001, when compared to the untreated vehicle group. In FIGS. 10A and 10B, combination therapies were compared to Compound A alone in a Dunnett's test one-factor linear model where &&=P<0.01. In FIG. 10A, combination therapies were compared to the respective additional active agent in a Dunnett's test one-factor linear model where \hat{ }\hat{ }\hat{ }=P<0.001.

### Additional Markers of Hepatic Fibrosis

FIG. 11A shows the fractional area of collagen-1 (Col1a1) expression for the various experimental groups.

FIG. 11B shows fractional area of relative α-smooth muscle actin (α-SMA) expression for the various experimental groups. The combination therapy Compound A+OCA significantly decreased liver α-SMA compared to the untreated vehicle group and compared to Compound A alone.

In FIGS. 11A and 11B, values are expressed as mean of n=9-12+SEM in a Dunnett's test one-factor linear model where **=P<0.01 and *** P<0.001, when compared to the untreated vehicle group. Combination therapies were compared to Compound A alone in a Dunnett's test one-factor linear model where &=P<0.05 and &&=P<0.01.

### Fibrosis Stage

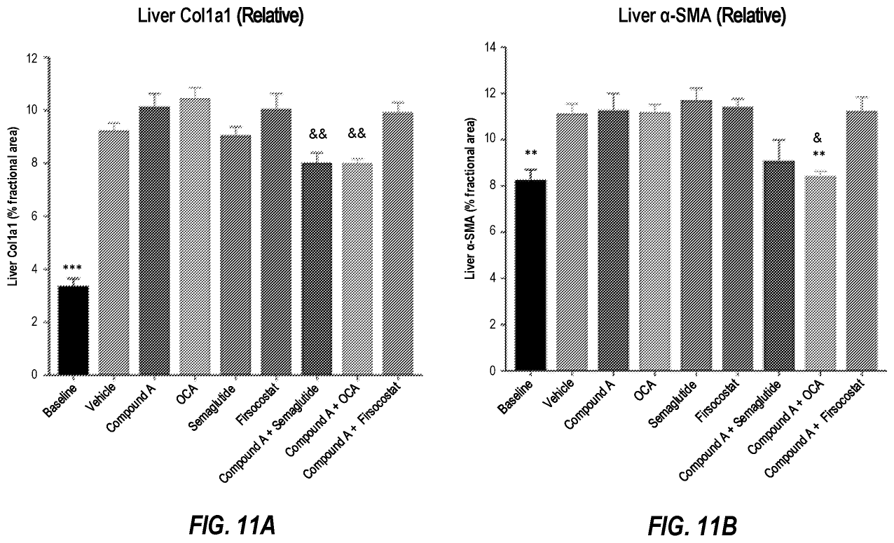

FIG. 12 shows the fibrosis stage scoring for each of the treatment groups. For each group, the number of animals with each score is indicated by the height of the stacked bars in a Mann-Whitney U test with Bonferroni correction where ***=P<0.001, when compared to the untreated vehicle group.

### Biological Example 12. Liver Morphology

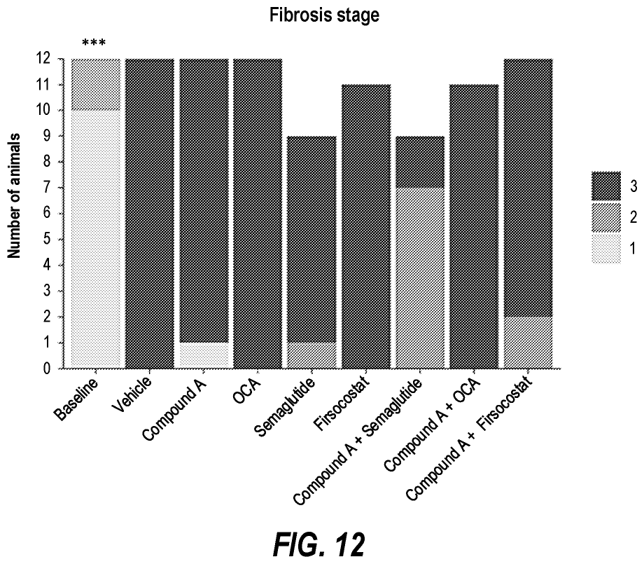

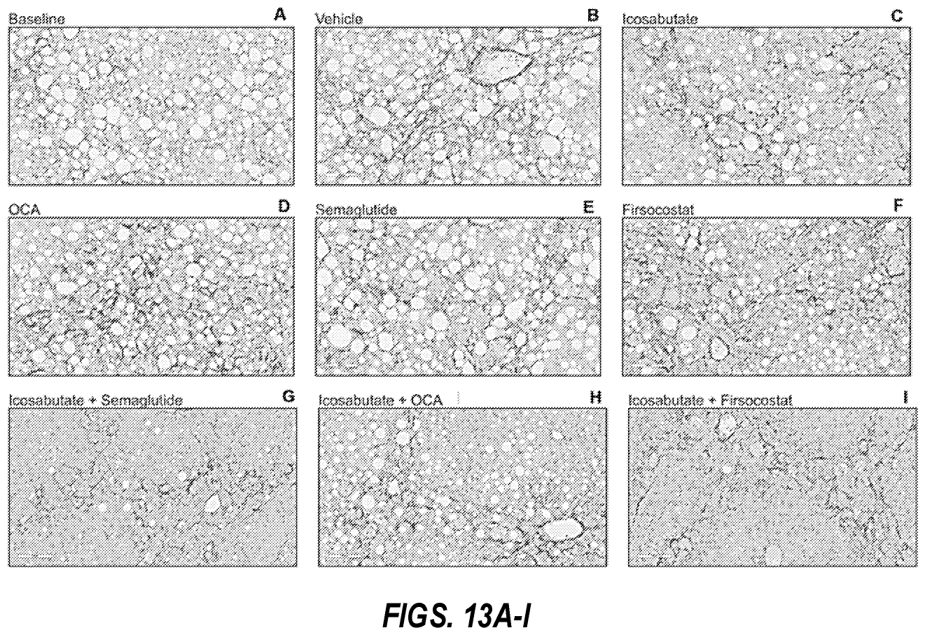

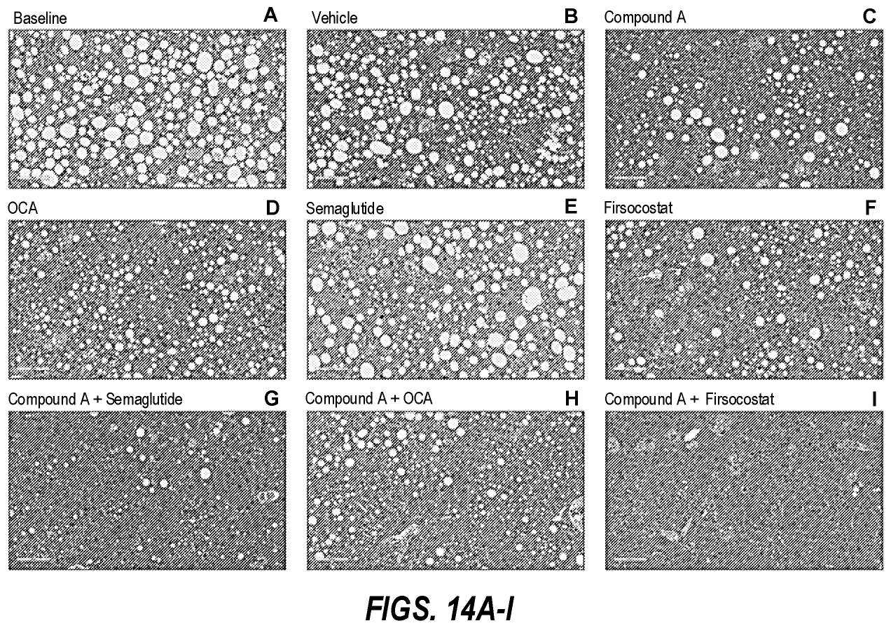

FIGS. 13A-131 show representative images of liver morphology of the various treatment groups at termination when stained with Picro Sirius Red. FIGS. 14A-14I show representative images of liver morphology of the various treatment groups at termination with H&E staining. The images were taken at a 20× magnification.

1. A combination therapy comprising a first compound of Formula (II) 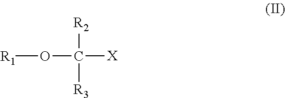
wherein R₁ is selected from a C₁₀-C₂₂ alkenyl having 3-6 double bonds;
R₂ and R₃ are the same or different and are selected from the group of a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group; wherein R₂ and R₃ can be connected in order to form a cycloalkane like cyclopropane, cyclobutane, cyclopentane or cyclohexane;
X is a carboxylic acid or a derivative thereof; wherein the derivative is a carboxylate, such as a carboxylic ester; a glyceride; an anhydride; a carboxamide; a phospholipid; or a hydroxymethyl; or a prodrug thereof;
or a pharmaceutically acceptable salt, solvate, or solvate of such a salt thereof, and at least one additional active agent chosen from a glucagon-like peptide 1 (GLP-1) receptor agonist, an acetyl-CoA carboxylase (ACC) inhibitor, and a farnesoid X receptor (FXR) agonist,
for use in therapeutic and/or prophylactic treatment of non-alcoholic steatohepatitis (NASH).

2. A combination therapy according to claim 1 for use according to claim 1 wherein the first compound is of Formula (I), and R₂, R₃, and X are as defined for Formula (II)

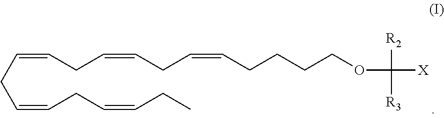

3. A combination therapy according to claim 1 or 2 for use according to claim 1
wherein for the first compound R₂ and R₃ are independently chosen from a hydrogen atom or linear, branched, and/or cyclic C₁-C₆ alkyl groups;
X is a carboxylic acid or a derivative thereof; wherein the derivative is a carboxylate, such as a carboxylic ester; a glyceride; an anhydride; a carboxamide; a phospholipid; or a hydroxymethyl; or a prodrug thereof;
or a pharmaceutically acceptable salt, solvate, or solvate of such salt thereof,
for use in treating non-alcoholic steatohepatitis.

4. The combination therapy according to any one of claims 1 to 3 for use according to claim 1, wherein for the first compound R₂ and R₃ are independently chosen from a hydrogen atom, a methyl group, an ethyl group, a n-propyl group, and an isopropyl group.

5. The combination therapy according to any one of claims 1 to 3 for use according to claim 1, wherein for the first compound R₂ and R₃ are both independently C₁-C₆ alkyl groups.

6. The combination therapy according to any one of claims 1 to 4 for use according to claim 1, wherein for the first compound one of R₂ and R₃ is a hydrogen atom and the other is an ethyl group.

7. The combination therapy according to any one of claims 1 to 6 for use according to claim 1, wherein for the first compound X is a carboxylic acid.

8. The combination therapy according to any one of claims 1 to 6 for use according to claim 1, wherein for the first compound X is a C₁-C₆ alkyl ester.

9. The combination therapy according to claim 8 for use according to claim 1, wherein for the first compound X is chosen from a methyl ester, an ethyl ester, an isopropyl ester, a n-butyl ester, and a tert-butyl ester.

10. The combination therapy according to any one of claim 1, 8, or 9 for use according to claim 1, wherein for the first compound X is selected from the group of a methyl ester and an ethyl ester.

11. The combination therapy according to any one of claims 1 to 6 for use according to claim 1, wherein for the first compound X is a glyceride chosen from a triglyceride, a 1,2-diglyceride, a 1,3-diglyceride, a 1-monoglyceride, and 2-monoglyceride.

12. The combination therapy according to any one of claims 1 to 11 for use according to claim 1, wherein the first compound is present in the form of an enantiomer, diastereomer, or mixture thereof.

13. The combination therapy according to claim 12 for use according to claim 1, wherein the first compound is present in its R form, in its S form or in racemic form.

14. The combination therapy according to claim 1 or 2 for use according to claim 1, wherein the first compound is 2-(((5Z,8Z,11Z,14Z,17Z)-icosa-5,8,11,14,17-pentaen-1-yl)oxy)butanoic acid (Compound A), or a pharmaceutically acceptable salt or ester thereof, and the formula is

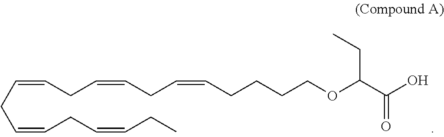 [SMILES: CC/C=C\C/C=C\C/C=C\C/C=C\C/C=C\CCCCOC(CC)C(=O)O]

15. The combination therapy according to any one of claims 1 to 14 for use according to claim 1, wherein the first compound is administered in a dose of between about 5 mg to about 4 g per dose.

16. The combination therapy according to any one of claims 1 to 15 for use according to claim 1, wherein the first compound is administered once daily.

17. The combination therapy according to any one of the preceding claims for use according to claim 1, wherein the additional active agent is chosen from semaglutide, firsocostat, and obeticholic acid (OCA).

18. The combination therapy according to any one of the preceding claims for use according to claim 1, wherein the additional active agent is semaglutide.

19. The combination therapy according to any one of the preceding claims for use according to claim 1, wherein the additional active agent is firsocostat.

20. The combination therapy according to any one of the preceding claims wherein the use treats or reverses NASH.

21. The combination therapy according to any one of the preceding claims wherein the use reduces the development of hepatic fibrosis or reduces existing hepatic fibrosis in a subject with NASH.

22. The combination therapy according to any one of the preceding claims wherein the use reduces the development of hepatic inflammation or reduces existing hepatic inflammation in a subject with NASH.

23. The combination therapy according to any one of the preceding claims wherein the use reduces the development of steatohepatitis or reduces existing steatohepatitis in a subject with NASH.

24. A combination therapy comprising a first compound of Formula (II) 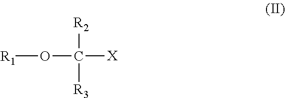
wherein R₁ is selected from a C₁₀-C₂₂ alkenyl having 3-6 double bonds;
R₂ and R₃ are the same or different and are selected from the group of a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group; wherein R₂ and R₃ can be connected in order to form a cycloalkane like cyclopropane, cyclobutane, cyclopentane or cyclohexane;
X is a carboxylic acid or a derivative thereof; wherein the derivative is a carboxylate, such as a carboxylic ester; a glyceride; an anhydride; a carboxamide; a phospholipid; or a hydroxymethyl; or a prodrug thereof;
or a pharmaceutically acceptable salt, solvate, or solvate of such a salt thereof, and at least one additional active agent chosen from a glucagon-like peptide 1 (GLP-1) receptor agonist, an acetyl-CoA carboxylase (ACC) inhibitor, and a farnesoid X receptor (FXR) agonist,
for use in therapeutic and/or prophylactic treatment of alcoholic steatohepatitis (ASH).

25. A combination therapy according to claim 24 for use according to claim 24 wherein the first compound is of Formula (I), and R₂, R₃, and X are as defined for Formula (II)

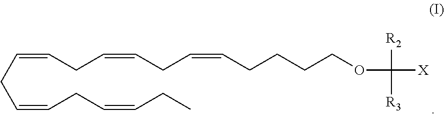

26. A combination therapy according to claim 24 or 25 for use according to claim 24
wherein for the first compound R₂ and R₃ are independently chosen from a hydrogen atom or linear, branched, and/or cyclic C₁-C₆ alkyl groups;
X is a carboxylic acid or a derivative thereof; wherein the derivative is a carboxylate, such as a carboxylic ester; a glyceride; an anhydride; a carboxamide; a phospholipid; or a hydroxymethyl; or a prodrug thereof;
or a pharmaceutically acceptable salt, solvate, or solvate of such salt thereof, for use in treating non-alcoholic steatohepatitis.

27. The combination therapy according to any one of claims 24 to 26 for use according to claim 24, wherein for the first compound R₂ and R₃ are independently chosen from a hydrogen atom, a methyl group, an ethyl group, a n-propyl group, and an isopropyl group.

28. The combination therapy according to any one of claims 24 to 26 for use according to claim 24, wherein for the first compound R₂ and R₃ are both independently C₁-C₆ alkyl groups.

29. The combination therapy according to any one of claims 24 to 27 for use according to claim 24, wherein for the first compound one of R₂ and R₃ is a hydrogen atom and the other is an ethyl group.

30. The combination therapy according to any one of claims 24 to 29 for use according to claim 24, wherein for the first compound X is a carboxylic acid.

31. The combination therapy according to any one of claims 24 to 29 for use according to claim 24, wherein for the first compound X is a C₁-C₆ alkyl ester.

32. The combination therapy according to claim 31 for use according to claim 24, wherein for the first compound X is chosen from a methyl ester, an ethyl ester, an isopropyl ester, a n-butyl ester, and a tert-butyl ester.

33. The combination therapy according to any one of claims 24, 31 and 32 for use according to claim 24, wherein for the first compound X is selected from the group of a methyl ester and an ethyl ester.

34. The combination therapy according to any one of claims 24 to 29 for use according to claim 24, wherein for the first compound X is a glyceride chosen from a triglyceride, a 1,2-diglyceride, a 1,3 diglyceride, a 1-monoglyceride, and 2-monoglyceride.

35. The combination therapy according to any one of claims 24 to 34 for use according to claim 24, wherein the first compound is present in the form of an enantiomer, diastereomer, or mixture thereof.

36. The combination therapy according to claim 35 for use according to claim 24, wherein the first compound is present in its R form, in its S form or in racemic form.

37. The combination therapy according to claim 24 or 25 for use according to claim 24, wherein the first compound is 2-(((5Z,8Z,11Z,14Z,17Z)-icosa-5,8,11,14, 17-pentaen-1-yl)oxy)butanoic acid (Compound A), or a pharmaceutically acceptable salt or ester thereof, and the formula is

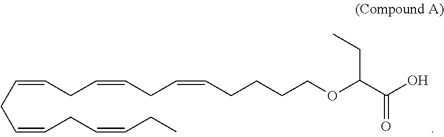 [SMILES: CC/C=C\C/C=C\C/C=C\C/C=C\C/C=C\CCCCOC(CC)C(=O)O]

38. The combination therapy according to any one of claims 24 to 37 for use according to claim 24, wherein the first compound is administered in a dose of between about 5 mg to about 4 g per dose.

39. The combination therapy according to any one of claims 24 to 38 for use according to claim 24, wherein the first compound is administered once daily.

40. The combination therapy according to any one of the preceding claims for use according to claim 24, wherein the additional active agent is chosen from semaglutide, firsocostat, and obeticholic acid (OCA).

41. The combination therapy according to any one of the preceding claims for use according to claim 24, wherein the additional active agent is semaglutide.

42. The combination therapy according to any one of the preceding claims for use according to claim 24, wherein the additional active agent is firsocostat.

43. The combination therapy according to any one of claims 24 to 42 wherein the use treats or reverses ASH.

44. The combination therapy according to any one of claims 24 to 43 wherein the use reduces the development of hepatic fibrosis or reduces existing hepatic fibrosis in a subject with ASH.

45. The combination therapy according to any one of claims 24 to 44 wherein the use reduces the development of hepatic inflammation or reduces existing hepatic inflammation in a subject with ASH.

46. The combination therapy according to any one of claims 24 to 45 wherein the use reduces the development of steatohepatitis or reduces existing steatohepatitis in a subject with ASH.

47. A combination therapy according to any of the preceding claims for use according to claim 1 or 24, wherein the first compound is formulated in a composition.

48. A combination therapy according to claim 47, for use according to claim 1, wherein the composition is formulated for oral administration.

49. The combination therapy according to any one of claims 47 to 48 for use according to claim 1 or 24, wherein the pharmaceutical composition further comprises at least one binder, excipient, diluent, or antioxidant or any combinations thereof.

50. The composition according to any one of claims 47 to 49 for use according to claim 1 or 24, wherein the compound is 2-(((5Z,8Z,11Z,14Z,17Z)-icosa-5,8,11,14,17-pentaen-1-yl)oxy)butanoic acid.

51. A method of treating non-alcoholic steatohepatitis (NASH) and/or alcoholic steatohepatitis (ASH) in a subject in need thereof, comprising administering to the subject a pharmaceutically effective amount of a first compound of formula (II): 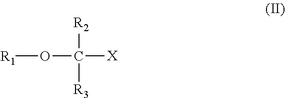
wherein R₁ is selected from a C₁₀-C₂₂ alkenyl having 3-6 double bonds;
R₂ and R₃ are the same or different and are selected from the group of a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group; wherein R₂ and R₃ can be connected in order to form a cycloalkane like cyclopropane, cyclobutane, cyclopentane or cyclohexane;
X is a carboxylic acid or a derivative thereof; wherein the derivative is a carboxylate, such as a carboxylic ester; a glyceride; an anhydride; a carboxamide; a phospholipid; or a hydroxymethyl; or a prodrug thereof;
or a pharmaceutically acceptable salt, solvate, or solvate of such a salt, thereof; and a pharmaceutically effective amount of at least one additional active agent chosen from a glucagon-like peptide 1 (GLP-1) receptor agonist, an acetyl-CoA carboxylase (ACC) inhibitor, and a farnesoid X receptor (FXR) agonist.

52. The method according to claim 51, wherein the first compound is of Formula (I):

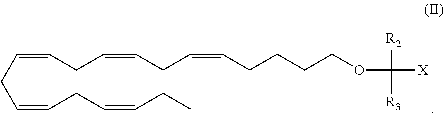

53. The method according to claim 51 or 52,
wherein for the first compound R₂ and R₃ are independently chosen from a hydrogen atom or linear, branched, and/or cyclic C₁-C₆ alkyl groups; and
X is a carboxylic acid or a derivative thereof; wherein the derivative is a carboxylate, such as a carboxylic ester; a glyceride; an anhydride; a carboxamide; a phospholipid; or a hydroxymethyl; or a prodrug thereof;
or a pharmaceutically acceptable salt, solvate, or solvate of such a salt, thereof.

54. The method according to any one of claims 51 to 53, wherein for the first compound R₂ and R₃ are independently chosen from a hydrogen atom, a methyl group, an ethyl group, an n-propyl group, and an isopropyl group.

55. The method according to any one of claims 51 to 54, wherein for the first compound R₂ and R₃ are both independently C₁-C₆ alkyl groups.

56. The method according to any one of claims 51 to 55, wherein for the first compound one of R₂ and R₃ is a hydrogen atom and the other is an ethyl group.

57. The method according to any one of claims 51 to 56, wherein for the first compound X is a carboxylic acid.

58. The method according to any one of claims 51 to 56, wherein for the first compound X is a C₁-C₆ alkyl ester.

59. The method according to claim 51, wherein for the first compound X is chosen from a methyl ester, an ethyl ester, an isopropyl ester, a n-butyl ester, and a tert-butyl ester.

60. The method according to any one of claim 51, 58, or 59 wherein for the first compound X is chosen from a methyl ester and an ethyl ester.

61. The method according to any one of claims 51 to 56, wherein for the first compound X is a glyceride chosen from a triglyceride, a 1,2-diglyceride, a 1,3-diglyceride, a 1-monoglyceride, and 2-monoglyceride.

62. The method according to any one of claims 51 to 61, wherein the first compound is present in the form of an enantiomer, diastereomer, or mixture thereof.

63. The method according to claim 62, wherein the first compound is present in its R form, in its S form, or in racemic form.

64. The method according to claim 51, wherein the first compound is 2-(((5Z,8Z,11Z,14Z,17Z)-icosa-5,8,11,14,17-pentaen-1-yl)oxy)butanoic acid (Compound A), or a pharmaceutically acceptable salt or ester thereof, and the formula is

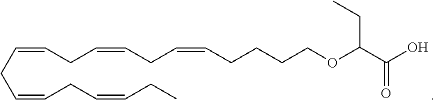 [SMILES: CC/C=C\C/C=C\C/C=C\C/C=C\C/C=C\CCCCOC(CC)C(=O)O]

65. The method according to any one of claims 51 to 64, wherein said compound is administered in a dose of between about 5 mg to about 4 g per dose.

66. The method according to any one of claims 51 to 65, wherein the compound is administered once daily.

67. The method according to any one of claims 51 to 66, wherein the additional active agent is chosen from semaglutide, firsocostat, and obeticholic acid (OCA).

68. The method according to any of claims 51 to 67, wherein the additional active agent is semaglutide.

69. The method according to any one of claims 51 to 67, wherein the additional active agent is firsocostat.

70. The method according to any one of claims 51 to 69, wherein the method treats or reverses NASH and/or ASH.

71. The method according to any one of claims 51 to 70, wherein the method comprises prophylactically treating NASH and/or ASH.

72. The method according to any one of claims 51 to 71, wherein the method reduces or prophylactically treats the development of hepatic fibrosis or reduces existing hepatic fibrosis.

73. The method according to any one of claims 51 to 72, wherein the use reduces the development of steatohepatitis or reduces existing steatohepatitis.

74. The method according to any one of claims 51 to 73, wherein the first compound is formulated as a pharmaceutical composition.

75. The method according to claim 74, wherein the pharmaceutical composition is formulated for oral administration.

76. The method according to claim 74 or 75, wherein the pharmaceutical composition further comprises at least one binder, excipient, diluent, or antioxidant, or any combination thereof.

77. The method according to any one of claims 51 to 76, wherein the first compound is 2-(((5Z,8Z,11Z,14Z,17Z)-icosa-5,8,11,14, 17-pentaen-1-yl)oxy)butanoic acid.

78. The method according to any one of claims 51 to 77, wherein the method of treatment is prophylactic.

79. The method according to any one of claims 51 to 78, wherein the first compound and at least one additional active agent are co-administered by simultaneous administration, sequential administration, overlapping administration, interval administration, continuous administration, or a combination thereof.

80. The method according to any one of claims 51 to 79, further comprising co-administering at least one further additional active agent.

81. The combination therapies and methods according to any one of the preceding claims, wherein the first compound is administered at a dosage of about 600 mg daily.

82. The combination therapies and methods according to any one of the preceding claims, wherein the compound is administered at a dosage of about 300 mg daily.

83. The combination therapies and methods according to any one of the preceding claims, wherein hepatic inflammatory cells are reduced by 30-50%.

84. The combination therapies and methods according to any one of the preceding claims, wherein hepatic inflammatory cells are reduced by 60-80%.

85. The combination therapies and methods according to any one of the preceding claims, wherein hepatic steatosis area is reduced by 70-90%.

86. The combination therapies and methods according to any one of the preceding claims, wherein the percentage of hepatocytes with lipid droplets is reduced by 70-90%.

87. The combination therapies and methods according to any one of the preceding claims, wherein the NAFLD activity (NAS) score is reduced.

88. The combination therapies and methods according to any one of the preceding claims, wherein the steatosis score is reduced.

89. The combination therapies and methods according to any one of the preceding claims, wherein the liver hydroxyproline content is decreased by 20-40%.

90. The combination therapies and methods according to any one of the preceding claims, wherein the hepatic fibrotic area as determined by picrosirius red staining is decreased by 30-50%.

91. A combination therapy comprising a first compound of Formula (II) 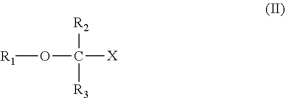
wherein R₁ is selected from a C₁₀-C₂₂ alkenyl having 3-6 double bonds;
R₂ and R₃ are the same or different and are selected from the group of a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group; wherein R₂ and R₃ can be connected in order to form a cycloalkane like cyclopropane, cyclobutane, cyclopentane or cyclohexane;
X is a carboxylic acid or a derivative thereof; wherein the derivative is a carboxylate, such as a carboxylic ester; a glyceride; an anhydride; a carboxamide; a phospholipid; or a hydroxymethyl; or a prodrug thereof;
or a pharmaceutically acceptable salt, solvate, or solvate of such a salt thereof, and at least one additional active agent chosen from a glucagon-like peptide 1 (GLP-1) receptor agonist, an acetyl-CoA carboxylase (ACC) inhibitor, and a farnesoid X receptor (FXR) agonist,
for use in therapeutic and/or prophylactic treatment of fatty liver disease.

92. A combination therapy according to claim 91 for use according to claim 91 wherein the first compound is of Formula (I), and R₂, R₃, and X are as defined for Formula (II)

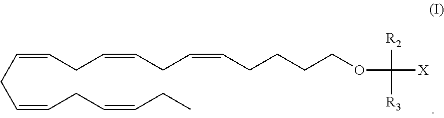

93. A combination therapy according to claim 91 or 92 for use according to claim 91 wherein for the first compound R₂ and R₃ are independently chosen from a hydrogen atom or linear, branched, and/or cyclic C₁-C₆ alkyl groups;
X is a carboxylic acid or a derivative thereof; wherein the derivative is a carboxylate, such as a carboxylic ester; a glyceride; an anhydride; a carboxamide; a phospholipid; or
a hydroxymethyl; or a prodrug thereof;
or a pharmaceutically acceptable salt, solvate, or solvate of such salt thereof.

94. The combination therapy according to any one of claims 91 to 93 for use according to claim 91, wherein for the first compound R₂ and R₃ are independently chosen from a hydrogen atom, a methyl group, an ethyl group, a n-propyl group, and an isopropyl group.

95. The combination therapy according to any one of claims 91 to 94 for use according to claim 91, wherein for the first compound R₂ and R₃ are both independently C₁-C₆ alkyl groups.

96. The combination therapy according to any one of claims 91 to 95 for use according to claim 91, wherein for the first compound one of R₂ and R₃ is a hydrogen atom and the other is an ethyl group.

97. The combination therapy according to any one of claims 91 to 96 for use according to claim 91, wherein for the first compound X is a carboxylic acid.

98. The combination therapy according to any one of claims 91 to 96 for use according to claim 91, wherein for the first compound X is a C₁-C₆ alkyl ester.

99. The combination therapy according to claim 98 for use according to claim 91, wherein for the first compound X is chosen from a methyl ester, an ethyl ester, an isopropyl ester, a n-butyl ester, and a tert-butyl ester.

100. The combination therapy according to any one of claims 91, 98 and 99 for use according to claim 91, wherein for the first compound X is selected from the group of a methyl ester and an ethyl ester.

101. The combination therapy according to any one of claims 91 to 96 for use according to claim 91, wherein for the first compound X is a glyceride chosen from a triglyceride, a 1,2-diglyceride, a 1,3-diglyceride, a 1-monoglyceride, and 2-monoglyceride.

102. The combination therapy according to any one of claims 91 to 101 for use according to claim 91, wherein the first compound is present in the form of an enantiomer, diastereomer, or mixture thereof.

103. The combination therapy according to claim 102 for use according to claim 91, wherein the first compound is present in its R form, in its S form or in racemic form.

104. The combination therapy according to claim 91 or 92 for use according to claim 91, wherein the first compound is 2-(((5Z,8Z,11Z,14Z,17Z)-icosa-5,8,11,14, 17-pentaen-1-yl)oxy)butanoic acid (Compound A), or a pharmaceutically acceptable salt or ester thereof, and the formula is

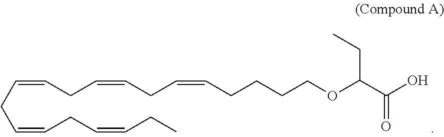 [SMILES: CC/C=C\C/C=C\C/C=C\C/C=C\C/C=C\CCCCOC(CC)C(=O)O]

105. The combination therapy according to any one of claims 91 to 104 for use according to claim 91, wherein the first compound is administered in a dose of between about 5 mg to about 4 g per dose.

106. The combination therapy according to any one of claims 91 to 105 for use according to claim 91, wherein the first compound is administered once daily.

107. The combination therapy according to any one of claims 91 to 106 for use according to claim 91, wherein the additional active agent is chosen from semaglutide, firsocostat, and obeticholic acid (OCA).

108. The combination therapy according to any one of claims 91 to 106 for use according to claim 91, wherein the additional active agent is semaglutide.

109. The combination therapy according to any one of claims 91 to 106 for use according to claim 91, wherein the additional active agent is firsocostat.

110. The combination therapy according to any one of claims 91 to 109 wherein the use treats or reverses fatty liver disease.

111. The combination therapy according to any one of claims 91 to 110, wherein the use reduces or reverses steatosis.

112. The combination therapy according to any one of claims 91 to 111, wherein the fatty liver disease is non-alcoholic fatty liver disease (NAFLD).

113. The combination therapy according to any one of claims 91 to 111, wherein the fatty liver disease is alcoholic fatty liver disease (ALD).

114. The combination therapy according to any one of claims 91 to 113, wherein the fatty liver disease is not accompanied by an inflammatory response and cellular damage.

115. The combination therapy according to any one of claims 91 to 114 for use according to claim 91, wherein the compound is 2-(((5Z,8Z,11Z,14Z,17Z)-icosa-5,8,11,14,17-pentaen-1-yl)oxy)butanoic acid.

116. A method of treating fatty liver disease in a subject in need thereof, comprising administering to the subject a pharmaceutically effective amount of a first compound of formula (II): 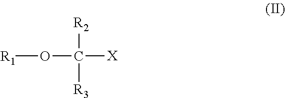
wherein R₁ is selected from a C₁₀-C₂₂ alkenyl having 3-6 double bonds;
R₂ and R₃ are the same or different and are selected from the group of a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group; wherein R₂ and R₃ can be connected in order to form a cycloalkane like cyclopropane, cyclobutane, cyclopentane or cyclohexane;
X is a carboxylic acid or a derivative thereof; wherein the derivative is a carboxylate, such as a carboxylic ester; a glyceride; an anhydride; a carboxamide; a phospholipid; or a hydroxymethyl; or a prodrug thereof;
or a pharmaceutically acceptable salt, solvate, or solvate of such a salt, thereof; and a pharmaceutically effective amount of at least one additional active agent chosen from a glucagon-like peptide 1 (GLP-1) receptor agonist, an acetyl-CoA carboxylase (ACC) inhibitor, and a farnesoid X receptor (FXR) agonist.

117. The method according to claim 116, wherein the first compound is of Formula (I):

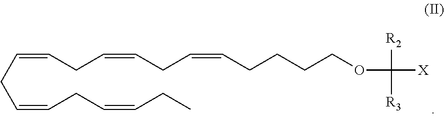

118. The method according to claim 116 or 117,
wherein for the first compound R₂ and R₃ are independently chosen from a hydrogen atom or linear, branched, and/or cyclic C₁-C₆ alkyl groups; and
X is a carboxylic acid or a derivative thereof; wherein the derivative is a carboxylate, such as a carboxylic ester; a glyceride; an anhydride; a carboxamide; a phospholipid; or
a hydroxymethyl; or a prodrug thereof;
or a pharmaceutically acceptable salt, solvate, or solvate of such a salt, thereof.

119. The method according to any one of claims 116 to 118, wherein for the first compound R₂ and R₃ are independently chosen from a hydrogen atom, a methyl group, an ethyl group, an n-propyl group, and an isopropyl group.

120. The method according to any one of claims 116 to 119, wherein for the first compound R₂ and R₃ are both independently C₁-C₆ alkyl groups.

121. The method according to any one of claims 116 to 120, wherein for the first compound one of R₂ and R₃ is a hydrogen atom and the other is an ethyl group.

122. The method according to any one of claims 116 to 121, wherein for the first compound X is a carboxylic acid.

123. The method according to any one of claims 51 to 121, wherein for the first compound X is a C₁-C₆ alkyl ester.

124. The method according to claim 116, wherein for the first compound X is chosen from a methyl ester, an ethyl ester, an isopropyl ester, a n-butyl ester, and a tert-butyl ester.

125. The method according to any one of claim 116, 123, or 124 wherein for the first compound X is chosen from a methyl ester and an ethyl ester.

126. The method according to any one of claims 116 to 121, wherein for the first compound X is a glyceride chosen from a triglyceride, a 1,2-diglyceride, a 1,3 diglyceride, a 1-monoglyceride, and 2-monoglyceride.

127. The method according to any one of claims 116 to 61, wherein the first compound is present in the form of an enantiomer, diastereomer, or mixture thereof.

128. The method according to claim 127, wherein the first compound is present in its R form, in its S form, or in racemic form.

129. The method according to claim 116, wherein the first compound is 2-(((5Z,8Z,11Z,14Z,17Z)-icosa-5,8,11,14, 17-pentaen-1-yl)oxy)butanoic acid (Compound A), or a pharmaceutically acceptable salt or ester thereof, and the formula is

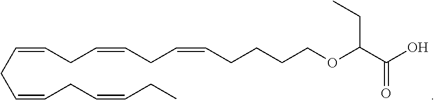 [SMILES: CC/C=C\C/C=C\C/C=C\C/C=C\C/C=C\CCCCOC(CC)C(=O)O]

130. The method according to any one of claims 116 to 129, wherein said compound is administered in a dose of between about 5 mg to about 4 g per dose.

131. The method according to any one of claims 116 to 130, wherein the compound is administered once daily.

132. The method according to any one of claims 116 to 131, wherein the additional active agent is chosen from semaglutide, firsocostat, and obeticholic acid (OCA).

133. The method according to any of claims 116 to 132, wherein the additional active agent is semaglutide.

134. The method according to any one of claims 116 to 132, wherein the additional active agent is firsocostat.

135. The method according to any one of claims 116 to 134, wherein the method treats or reverses fatty liver disease.

136. The method according to any one of claims 116 to 134, wherein the method comprises prophylactically treating fatty liver disease.

137. The method according to any one of claims 116 to 136, wherein the fatty liver disease is non-alcoholic fatty liver disease (NAFLD).

138. The method according to any one of claims 116 to 136, wherein the fatty liver disease is alcoholic fatty liver disease (ALD).

139. The method according to any one of claims 116 to 138, wherein the fatty liver disease is not accompanied by an inflammatory response and cellular damage.

140. The method according to any one of claim 139, wherein the first compound is 2-(((5Z,8Z,11Z,14Z,17Z)-icosa-5,8,11,14,17-pentaen-1-yl)oxy)butanoic acid.

141. The combination therapies and methods according to any one claims 91 to 140, wherein the first compound is administered at a dosage of about 600 mg daily.

142. The combination therapies and methods according to any one of claims 91 to 141, wherein the compound is administered at a dosage of about 300 mg daily.

143. The combination therapies and methods according to any one of claims 91 to 142, wherein hepatic steatosis area is reduced by 70-90%.

144. The combination therapies and methods according to any one of claims 91 to 143, wherein the percentage of hepatocytes with lipid droplets is reduced by 70-90%.

145. The combination therapies and methods according to any one of claims 91 to 144, wherein the NAFLD activity (NAS) score is reduced.

146. The combination therapies and methods according to any one of claims 91 to 145, wherein the steatosis score is reduced.
